SEQUENCE LISTING (70 sequences)

SEQ ID NO: 1 - protein, 16 aa, Artificial Sequence
  Synthesized
KSSQSLLHSDGKTYLN

SEQ ID NO: 2 - protein, 7 aa, Artificial Sequence
  Synthesized
LVSRLDS

SEQ ID NO: 3 - protein, 9 aa, Artificial Sequence
  Synthesized
WQGTHFPRT

SEQ ID NO: 4 - protein, 10 aa, Artificial Sequence
  Synthesized
GFYIKDTYIH

SEQ ID NO: 5 - protein, 10 aa, Artificial Sequence
  Synthesized
GIGSADGDTR

SEQ ID NO: 6 - protein, 10 aa, Artificial Sequence
  Synthesized
GIDPADGDTR

SEQ ID NO: 7 - protein, 4 aa, Artificial Sequence
  Synthesized
ELAY

SEQ ID NO: 8 - protein, 16 aa, Artificial Sequence
  Synthesized
KSSHSLLHRDGRTYLN

SEQ ID NO: 9 - protein, 7 aa, Artificial Sequence
  Synthesized
LVSKLDS

SEQ ID NO: 10 - protein, 9 aa, Artificial Sequence
  Synthesized
WQGTHFPRT

SEQ ID NO: 11 - protein, 10 aa, Artificial Sequence
  Synthesized
GLNIKDSYLH

SEQ ID NO: 12 - protein, 10 aa, Artificial Sequence
  Synthesized
GIDPANGDVE

SEQ ID NO: 13 - protein, 3 aa, Artificial Sequence
  Synthesized
FPY

SEQ ID NO: 14 - protein, 16 aa, Artificial Sequence
  Synthesized
RSSQSLVHSNGNTYLH

SEQ ID NO: 15 - protein, 7 aa, Artificial Sequence
  Synthesized
KASNRFS

SEQ ID NO: 16 - protein, 9 aa, Artificial Sequence
  Synthesized
FQSTHVPYT

SEQ ID NO: 17 - protein, 10 aa, Artificial Sequence
  Synthesized
GFNIKDTYMH

SEQ ID NO: 18 - protein, 10 aa, Artificial Sequence
  Synthesized
GIDPADGEPL

SEQ ID NO: 19 - protein, 7 aa, Artificial Sequence
  Synthesized
VRSSFDY

SEQ ID NO: 20 - protein, 16 aa, Artificial Sequence
  Synthesized
KSSQSLLHRDGKTYLN

SEQ ID NO: 21 - protein, 7 aa, Artificial Sequence
  Synthesized
LVSLVDS

SEQ ID NO: 22 - protein, 9 aa, Artificial Sequence
  Synthesized
WQGTHFPRT

SEQ ID NO: 23 - protein, 10 aa, Artificial Sequence
  Synthesized
GFNIKDTYMH

SEQ ID NO: 24 - protein, 10 aa, Artificial Sequence
  Synthesized
GIDPETGNTK

SEQ ID NO: 25 - protein, 7 aa, Artificial Sequence
  Synthesized
LGRPFAH

SEQ ID NO: 26 - protein, 16 aa, Artificial Sequence
  Synthesized
KSSHSLLHRDGRTYLN

SEQ ID NO: 27 - protein, 7 aa, Artificial Sequence
  Synthesized
LVSKLDS

SEQ ID NO: 28 - protein, 9 aa, Artificial Sequence
  Synthesized
WQGTHFPRT

SEQ ID NO: 29 - protein, 10 aa, Artificial Sequence
  Synthesized
GFSIRDTYMH

SEQ ID NO: 30 - protein, 10 aa, Artificial Sequence
  Synthesized
GIDPENGNSK

SEQ ID NO: 31 - protein, 4 aa, Artificial Sequence
  Synthesized
ELAY

SEQ ID NO: 32 - protein, 17 aa, Artificial Sequence
  Synthesized
KSSQSLLNSRTRKNYLA

SEQ ID NO: 33 - protein, 7 aa, Artificial Sequence
  Synthesized
WASTRES

SEQ ID NO: 34 - protein, 8 aa, Artificial Sequence
  Synthesized
KQSYNFIT

SEQ ID NO: 35 - protein, 10 aa, Artificial Sequence
  Synthesized
GYTFTVYVIH

SEQ ID NO: 36 - protein, 10 aa, Artificial Sequence
  Synthesized
YINPYNDGAE

SEQ ID NO: 37 - protein, 14 aa, Artificial Sequence
  Synthesized
GENYYTSRYGFFDV

SEQ ID NO: 38 - protein, 114 aa, Artificial Sequence
  Synthesized
DILMTQSPLNLSVTIGQPASISCKSSQSLLHSDGKTYLNWLLQRPGQSPKRLMYLVSRLDSGVPDRFTGSGSGTDFTLKISRVEAEDLGVYYCWQGTHFPRTFGGGTKLEIKRA

SEQ ID NO: 39 - protein, 112 aa, Artificial Sequence
  Synthesized
EVQLEESGAELVKPGASVKLSCTASGFYIKDTYIHWVKQRPEEVLEWIGGIDPADGDTRYDPKFQGKATITADTSSNSAYLHLTSLTSEDTAVYFCARELAYWGAGTTVTVS

SEQ ID NO: 40 - protein, 106 aa, Artificial Sequence
  Synthesized
EVQLEESGAELVKPGASVKLSCTASGFYIKDTYIHWVKQRPEEVLEWIGGIGSADGDTRYDPKFQGKATITADTSSNSAYLHLTSLTSEDTAVYFCARELAYWGAG

SEQ ID NO: 41 - protein, 106 aa, Artificial Sequence
  Synthesized
EVQLEESGAELVKPGASVKLSCTASGFYIKDTYIHWVKQRPEEVLEWIGGIDPADGDTRYDPKFQGKATITADTSSNSAYLHLTSLTSEDTVVYFCARELAYWGAG

SEQ ID NO: 42 - protein, 110 aa, Artificial Sequence
  Synthesized
EVQLEESGAELVKPGASVKLSCTASGFYIKDTYIHWVRQRPEEVLEWIGGIDPADGDTRYDPKFQGKATITADTSSNSAYLHLTSLTSEDTAVYFCARELAYWGAGTTVT

SEQ ID NO: 43 - protein, 112 aa, Artificial Sequence
  Synthesized
EVQLEESGAELVKPGASVKLSCTASGFYIKDTYIHWVKQRPEEVLEWIGGIDPADGDTRYDPKSQGKATITADTSSNSAYLHLTSLTSEDTAVYFCARELAYWGAGTTVTIT

SEQ ID NO: 44 - protein, 109 aa, Artificial Sequence
  Synthesized
EVQLEESGAELVKPGASVKLSCTASGFYIKDTYIHWVKQRPEEVLEWIGGIGSADGDTRYDPKFQGKATITADTSSNSAYLHLTSLTSEDTVVYFCARELAYWGAWTTV

SEQ ID NO: 45 - protein, 114 aa, Artificial Sequence
  Synthesized
DILMTQSPLTLSVIIGQPASFSCKSSHSLLHRDGRTYLNWLLQRPGQSPQRLIYLVSKLDSGVPDRFTGSGSGTDFTLKISRVEAEDLGVYYCWQGTHFPRTFGGGTKLEIKRA

SEQ ID NO: 46 - protein, 111 aa, Artificial Sequence
  Synthesized
EVQLEESGAEFVRPGAAVKLSCTVSGLNIKDSYLHWVKQRPEQGLEWIGGIDPANGDVEYDPKFQGKAAITADTSSNTAYLRLSSLTSEDTAVYYCAPFPYWGAGTTVTVS

SEQ ID NO: 47 - protein, 114 aa, Artificial Sequence
  Synthesized
CILMTQSPLSLPVSLGDQASISCRSSQSLVHSNGNTYLHWYLQKPGQSPKFLIYKASNRFSGVPDRFSGRGSGTDFTLKISRVEAEDLGVYFCFQSTHVPYTFGGGTKLEIKRA

SEQ ID NO: 48 - protein, 115 aa, Artificial Sequence
  Synthesized
EVKLQQSGAELVKPGASVKLSCTASGFNIKDTYMHWVKQRPEQGLEWIGGIDPADGEPLYDPKFQDKATITTDTSSNTVYLQISSLTSEDSPVYYCAPVRSSFDYWGQGTTVTVS

SEQ ID NO: 49 - protein, 101 aa, Artificial Sequence
  Synthesized
HSADPVSISCKSSQSLLHRDGKTYLNWVFQRPGQSPQRLIYLVSLVDSGVPDRFTGSGSGTDFTLKINRVEAEDLGVYYCWQGTHFPRTFGGGTKLEIKRA

SEQ ID NO: 50 - protein, 115 aa, Artificial Sequence
  Synthesized
EVQLQQSGAELAKPGASVKLSCTGSGFNIKDTYMHWVKQRPEQGLEWIGGIDPETGNTKFDPRFQDKATITSDTSSNTVLLQLSSLTSEDTAVYYCANLGRPFAHWGQGTTVTVS

SEQ ID NO: 51 - protein, 114 aa, Artificial Sequence; identical to SEQ ID NO: 45
  Synthesized

SEQ ID NO: 52 - protein, 112 aa, Artificial Sequence
  Synthesized
EVKLQQSGAELVKPGASVKLSCTASGFSIRDTYMHWVRQRPEQGLEWITGIDPENGNSKYAPRFQDKATIIADTSSNTVHLQLDTLTSEDTAVYYCTRELAYWGQGTTVTVS

SEQ ID NO: 53 - protein, 114 aa, Artificial Sequence
  Synthesized
DILMPQSPSSLAVSAGEKVTMSCKSSQSLLNSRTRKNYLAWYQQKLGQSPKLLIYWASTRESGVPDRFTGSGSGTDFTLTISSVQAEDLAVYYCKQSYNFITFGAGTKLELKRA

SEQ ID NO: 54 - protein, 122 aa, Artificial Sequence
  Synthesized
EVKLQESGPDLVKPGASVKVSCKASGYTFTVYVIHWVIQKPGQGLEWIGYINPYNDGAEYNENFKGKATLTSDKSSSTAYMELSSLTSEDSAVYYCTRGENYYTSRYGFFDVWGQGTTVTVS

SEQ ID NO: 55 - protein, 750 aa, Homo sapiens
  ref <308>: DATABASE ACCESSION NUMBER: NP_004467
  ref <309>: DATABASE ENTRY DATE: 1993-01-01
  ref <313>: RELEVANT RESIDUES IN SEQ ID NO: (1)..(750)
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 56 - protein, 12 aa, Artificial Sequence
  Synthesized
CGKSLYESWTKK

SEQ ID NO: 57 - DNA, 21 bp, Artificial Sequence
  Synthesized
tgaggtgcagctggaggagtc

SEQ ID NO: 58 - DNA, 24 bp, Artificial Sequence
  Synthesized
gtgaccgtggtccctgcgccccag

SEQ ID NO: 59 - DNA, 21 bp, Artificial Sequence
  Synthesized
gacattctgatgacccagtct

SEQ ID NO: 60 - DNA, 21 bp, Artificial Sequence
  Synthesized
ttttatttccagcttggtccc

SEQ ID NO: 61 - protein, 17 aa, Artificial Sequence
  Synthesized
  MISC_FEATURE (1)..(1): Xaa is a basic amino acid
  MISC_FEATURE (4)..(4): Xaa is glutamine or histidine
  MISC_FEATURE (7)..(7): Xaa is an hydrophobic amino acid
  MISC_FEATURE (8)..(8): Xaa is asparagine or histidine
  MISC_FEATURE (9)..(9): Xaa is serine or arginine
  MISC_FEATURE (10)..(10): Xaa is absent or arginine
  MISC_FEATURE (11)..(11): Xaa is aspartic acid, asparagine or threonine
  MISC_FEATURE (12)..(12): Xaa is glycine or arginine
  MISC_FEATURE (13)..(13): Xaa is a basic amino acid
  MISC_FEATURE (14)..(14): Xaa is threonine or asparagine
  MISC_FEATURE (17)..(17): Xaa is asparagine, histidine or alanine
XSSXSLXXXXXXXXYLX

SEQ ID NO: 62 - protein, 7 aa, Artificial Sequence
  Synthesized
  MISC_FEATURE (4)..(4): Xaa is a basic amino acid
  MISC_FEATURE (5)..(5): Xaa is an hydrophobic amino acid
  MISC_FEATURE (7)..(7): Xaa is serine or absent
LVSXXDX

SEQ ID NO: 63 - protein, 7 aa, Artificial Sequence
  Synthesized
  MISC_FEATURE (1)..(1): Xaa is lysine or tryptophan
  MISC_FEATURE (4)..(4): Xaa is asparagine or threonine
  MISC_FEATURE (6)..(6): Xaa is phenylalanine or glutamic acid
XASXRXS

SEQ ID NO: 64 - protein, 9 aa, Artificial Sequence
  Synthesized
  MISC_FEATURE (1)..(1): Xaa is an aromatic amino acid
  MISC_FEATURE (3)..(3): Xaa is serine or glycine
  MISC_FEATURE (6)..(6): Xaa is phenylalanine or valine
  MISC_FEATURE (8)..(8): Xaa is arginine or tyrosine
XQXTHXPXT

SEQ ID NO: 65 - protein, 10 aa, Artificial Sequence
  Synthesized
  MISC_FEATURE (2)..(2): Xaa is an hydrophobic amino acid
  MISC_FEATURE (3)..(3): Xaa is asparagine, serine, tyrosine or threonine
  MISC_FEATURE (4)..(4): Xaa is an hydrophobic amino acid
  MISC_FEATURE (5)..(5): Xaa is a basic amino acid
  MISC_FEATURE (6)..(6): Xaa is valine or aspartic acid
  MISC_FEATURE (7)..(7): Xaa is an hydrophilic amino acid
  MISC_FEATURE (8)..(8): Xaa is tyrosine or valine
  MISC_FEATURE (9)..(9): Xaa is an hydrophobic amino acid
GXXXXXXXXH

SEQ ID NO: 66 - protein, 10 aa, Artificial Sequence
  Synthesized
  MISC_FEATURE (2)..(2): Xaa is an hydrophobic amino acid
  MISC_FEATURE (3)..(3): Xaa is asparagine, serine or tyrosine
  MISC_FEATURE (5)..(5): Xaa is a basic amino acid
  MISC_FEATURE (7)..(7): Xaa is an hydrophilic amino acid
  MISC_FEATURE (9)..(9): Xaa is an hydrophobic amino acid
GXXIXDXYXH

SEQ ID NO: 67 - protein, 10 aa, Artificial Sequence
  Synthesized
  MISC_FEATURE (3)..(3): Xaa is aspartic acid or glycine
  MISC_FEATURE (4)..(4): Xaa is proline or serine
  MISC_FEATURE (5)..(5): Xaa is alanine or glutamic acid
  MISC_FEATURE (6)..(6): Xaa is threonine, asparagine or aspartic acid
  MISC_FEATURE (8)..(8): Xaa is aspartic acid, glutamic acid or asparagine
  MISC_FEATURE (9)..(9): Xaa is threonine, serine, valine or proline
  MISC_FEATURE (10)..(10): Xaa is a basic amino acid
GIXXXXGXXX

SEQ ID NO: 68 - protein, 10 aa, Artificial Sequence
  Synthesized
  MISC_FEATURE (6)..(6): Xaa is threonine or arginine
  MISC_FEATURE (9)..(9): Xaa is a neutral hydrophilic amino acid
GIDPEXGNXK

SEQ ID NO: 69 - protein, 115 aa, Artificial Sequence
  Synthesized
LGQLQQSGAELVKPGASVKLSCTGSGFNIKDTYMHWVKQRPEQGLEWIGGIDPENGNTKFDPRFQDKATITADASSNTVLLQLSSLTSEDTAVYYCANLGRPFAHWGQGTTVTSS

SEQ ID NO: 70 - protein, 10 aa, Artificial Sequence
  Synthesized
GIDPENGNTK

(19) United States
(12) Patent Application Publication
(10) Pub. No.: US 2011/0189093 A1
(43) Pub. Date: Aug. 4, 2011

(54) PROSTATE SPECIFIC MEMBRANE ANTIGEN ANTIBODIES AND ANTIGEN BINDING FRAGMENTS

(75) Inventors: Serge Moffett, Saint-Laurent (CA); Dominic Melancon, Blainville (CA); Uri H. Saragovi, Montreal (CA); Phil Gold, Westmount (CA); Claudio A. Cuello, Westmount (CA)

(73) Assignee: PROSCAN RX PHARMA, Montreal, QC (CA)

(21) Appl. No.: 12/937,684

(22) PCT Filed: Apr. 14, 2009
(86) PCT No.: PCT/CA2009/000470

§ 371 Date: Apr. 4, 2011

Related U.S. Application Data
(60) Provisional application No. 61/071,112, filed on Apr. 14, 2008.

(51) Int. Cl. A61K 49/00; C07K 16/30; A61K 39/395; A61P 35/00

(52) U.S. Cl. 424/9.1; 530/389.7; 424/174.1; 530/391.3; 530/391.7; 424/178.1

(57) ABSTRACT

Polypeptides, antibodies or antigen-binding fragments capable of binding to prostate specific membrane antigen (PSMA) are provided. These polypeptides, antibodies or antigen-binding fragments may be used for diagnostic and/or therapeutic purposes.

### FIELD OF INVENTION

The present invention relates to the field of antibodies (Ab) and to antigen binding fragments thereof. More specifically, the invention relates to diagnostic and therapeutic antibodies and antigen binding fragments capable of binding to prostate specific membrane antigen (PSMA).

### BACKGROUND OF INVENTION

Prostate cancer is the most commonly diagnosed nonskin malignancy in males from developed countries. It is estimated that one in six males will be diagnosed with prostate cancer (PCa) in their lifetime. The diagnosis of PCa has greatly improved following the use of serum-based markers such as the prostate-specific antigen (PSA). However, the use of tumor-associated markers offers alternative strategies in disease management and may prove useful for in vivo tumor imaging purposes and further development of targeted therapies.

Identification of the prostate specific membrane antigen (PSMA) marker, a tumor associated marker, has generated interest for both applications. PSMA is a glycoprotein highly restricted to prostate secretory epithelial cell membranes. Its expression has been correlated with tumor aggressiveness. Various immunohistological studies have demonstrated increased PSMA levels in virtually all cases of prostatic carcinoma compared to those levels in benign prostate epithelial cells. Intense PSMA staining is found in all stages of the disease, including prostatic intraepithelial neoplasia, late stage androgen-independent prostate cancer and secondary prostate tumors localized to lymph nodes, bone, soft tissue, and lungs. PSMA was originally identified as the molecule recognized by 7E11, a monoclonal antibody (MAb) reactive to the prostate cancer cell line LNCaP. It was subsequently cloned from these cells as a 2.65 kb cDNA encoding a 750 amino acid cell surface type II integral membrane glycoprotein of 100 kDa. PSMA forms a noncovalent homodimer that possesses glutamate carboxypeptidase activity based on its ability to process the neuropeptide N-acetylaspartylglutamate and glutamate-conjugated folate derivatives. Although the precise biological role played by PSMA in disease pathogenesis remains elusive, its overexpression in PCa might potentially be associated with the growth balance of the prostate gland. Indeed, recent evidence suggests that PSMA performs multiple physiological functions related to cell survival and migration.

Antibody-based therapeutics have emerged as important components of therapies for an increasing number of human malignancies in such fields as oncology, inflammatory and infectious diseases. In most cases, the basis of the therapeutic function is the high degree of specificity and affinity the antibody-based drug has for its target antigen. Arming monoclonal antibodies with drugs, toxins, or radionuclides is yet another strategy by which mAbs may induce therapeutic effect. By combining the exquisite targeting specificity of antibody with the tumor killing power of toxic effector molecules, immunoconjugates permit sensitive discrimination between target and normal tissue thereby resulting in fewer side effects than most conventional chemotherapeutic drugs.

Given the physical properties of PSMA and its expression pattern in relation to disease progression, its large extracellular domain provides an excellent target in the development of ligands for diagnostic and therapeutic intervention. The first PSMA-specific MAb reported, 7E11, was subsequently developed and commercialized as a diagnostic agent for tumor imaging (ProstaScint, Cytogen, Princeton, N.J.). However, this antibody recognizes an intracellular epitope of PSMA which limits its usefulness as an imaging agent for the detection of PSMA. More recently, MAbs such as J591 that recognize the extracellular portion of PSMA were developed, however such antibodies have uncharacterized epitope specificities. The development of improved anti-PSMA antibodies with diagnostic and/or therapeutic activity is needed. The present invention seeks to meet these and other needs.

### SUMMARY OF THE INVENTION

This invention relates to antibodies and antigen binding fragments, cells comprising or expressing these antibodies or antigen binding fragments as well as kits useful for the treatment, detection of tumor cells and neovasculature or in the diagnosis of cancer.

The Applicant came to the unexpected discovery that the antibodies and antigen binding fragments of the present invention does not need to be conjugated with a toxic or other therapeutic moiety in order to efficiently reduce the growth of cancer cells in vivo. In fact, these antibodies or antigen binding fragments are capable of inducing or promoting cell death of PSMA-expressing cells (especially PSMA-expressing tumor cells) by themselves. This represents a significant advantage over other antibodies known in the art.

The antibodies and antigen binding fragments of the present invention are particularly useful for reducing or inhibiting the growth of cancer cells. The antibodies and antigen binding fragments of the present invention may also be linked to a detectable moiety for detection and/or diagnostic purposes. Optionally, if so desired, these antibodies and antigen binding fragments may be linked to a therapeutic moiety. In an aspect of the invention, for therapeutic purposes, the naked antibody or antigen binding fragments may be unconjugated.

The present invention provides in one aspect thereof, an isolated or substantially purified antibody or antigen binding fragment which may be capable of specific binding to PSMA (SEQ ID NO:55). Since, the antibody or antigen binding fragment of the present invention may advantageously promote cell death indepentently of the presence of a cytotoxic molecule, they are referred herein as naked antibodies or antigen binding fragments thereof.

More specifically and in accordance with an embodiment of the invention, the antibody or antigen binding fragment may bind to a domain located between amino acids 490 and 500 of PSMA.

In accordance with another embodiment of the invention, the antibody or antigen binding fragment may be capable of binding to an epitope comprised within amino acid 490 and 500 of PSMA. In fact, the antibody or antigen binding fragment may be capable of binding to an epitope consisting of amino acids 490 to 500 (inclusively) of PSMA, i.e., SEQ ID NO.:56.

Also encompassed by the present invention are antibodies or antigen binding fragments having the same epitope specificity as the antibody of the present invention and having substantially the same activity, preferably substantially the same therapeutic activity. A candidate antibody may be identified by determining whether it will bind to the epitope to which the antibodies described herein binds and/or by performing competition assays with antibodies or antigen binding fragments known to bind to the epitope. A candidate antibody is preferably selected for its ability to reduce the growth of cancer cells without being conjugated to a toxin or to other therapeutic moiety.

Therefore another aspect the present invention provides an isolated antibody or antigen binding fragment capable of competing with the antibody or antigen binding fragment described herein.

In further aspects, the present invention provides methods of treatment and methods of detection using the antibody or antigen binding fragment of the present invention.

The term “antibody” refers to intact antibody, monoclonal or polyclonal antibodies. The term “antibody” also encompasses, multispecific antibodies such as bispecific antibodies. Human antibodies are usually made of two light chains and two heavy chains each comprising variable regions and constant regions. The light chain variable region comprises 3 CDRs, identified herein as CDRL1, CDRL2 and CDRL3 flanked by framework regions. The heavy chain variable region comprises 3 CDRs, identified herein as CDRH1, CDRH2 and CDRH3 flanked by framework regions.

The term “antigen-binding fragment”, as used herein, refers to one or more fragments of an antibody that retain the ability to bind to an antigen. It has been shown that the antigen-binding function of an antibody can be performed by fragments of an intact antibody. Examples of binding fragments encompassed within the term “antigen-binding fragment” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR), e.g., V_{H} CDR3 comprising or not additional sequence (linker, framework region(s) etc.) and (v) a combination of two to six isolated CDRs comprising or not additional sequence (linker framework region(s) etc.). Furthermore, although the two domains of the Fv fragment, V_{L} and V_{H}, are coded for by separate genes, they can be joined, using recombinant methods, by a synthetic linker that enables them to be made as a single polypeptide chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region may be modified by replacing one or more cysteine residues with serine residues so as to prevent dimerization. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

A typical antigen binding site is comprised of the variable regions formed by the pairing of a light chain immunoglobulin and a heavy chain immunoglobulin. The structure of the antibody variable regions is very consistent and exhibits very similar structures. These variable regions are typically comprised of relatively homologous framework regions (FR) interspaced with three hypervariable regions termed Complementarity Determining Regions (CDRs). The overall binding activity of the antigen binding fragment is often dictated by the sequence of the CDRs. The FRs often play a role in the proper positioning and alignment in three dimensions of the CDRs for optimal antigen binding.

In fact, because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998, Nature 332:323-327; Jones, P. et al., 1986, Nature 321:522-525; and Queen, C. et al., 1989, Proc. Natl. Acad. See. U.S.A. 86:10029-10033). Such framework sequences can be obtained from public DNA databases that include germline antibody gene sequences. These germline sequences will differ from mature antibody gene sequences because they will not include completely assembled variable genes, which are formed by V(D)J joining during B cell maturation. Germline gene sequences will also differ from the sequences of a high affinity secondary repertoire antibody which contains mutations throughout the variable gene but typically clustered in the CDRs. For example, somatic mutations are relatively infrequent in the amino terminal portion of framework region 1 and in the carboxy-terminal portion of framework region 4. Furthermore, many somatic mutations do not significantly alter the binding properties of the antibody. For this reason, it is not necessary to obtain the entire DNA sequence of a particular antibody in order to recreate an intact recombinant antibody having binding properties similar to those of the original antibody. Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. To add missing sequences, cloned cDNA sequences can be combined with synthetic oligonucleotides by ligation or PCR amplification. Alternatively, the entire variable region can be synthesized to create an entirely synthetic variable region clone. This process has certain advantages such as elimination or inclusion of particular restriction sites, or optimization of particular codons.

Of course, the totality or portions of the framework region of the antibody described herein may be used in conjunction with the CDRs in order to optimize the affinity, specificity or any other desired properties of the antibody.

The term “naked antibody or antigen binding fragment” refers to an antibody or antigen binding fragment which has the ability to induce cell death in vitro or in vivo, without needed to be conjugated with a toxin, drug or the like. The term “naked”, in some instances may also refer to an antibody or antigen binding fragment which is optionally conjugated with a moiety which is considered as being therapeutic.

Antibodies and/or antigen binding fragments of the present invention may originate, for example, from a mouse, a rat or any other mammal or from other sources such as through recombinant DNA technologies.

### BRIEF DESCRIPTION OF THE DRAWINGS

In the appended drawings which illustrates non-limitative, exemplary embodiments of the present invention:

FIG. 1 shows the sequences of the light and heavy chain variable regions of the antibodies of the present invention;

FIG. 2 shows a control-corrected sensorgram related to PSf42.2 injections over PSMA surfaces;

FIG. 3 shows immunoreactivity of PSf42.2 and PSf47.1 against benign and malignant prostatic tissue. A) Benign prostatic tissue immunostained with mAb PSf42.2; B) prostate cancer of Gleason score 3+3=6 showing immunolabeling with mAb PSf42.2; C) Prostate cancer of Gleason score 4+4=8 showing immunolabeling with mAb PSf47.1;

FIG. 4 shows immunoreactivity of PSf47.1 against small bowel and proximal renal tubules. Immunoreactivity with mAb PSf47.1 of A) duodenal brush border and B) proximal renal tubules;

FIGS. 5A and 5B are photographs of whole body gamma camera images of Ab-DOTA-In111 in experimental mouse model of prostate cancer. Mouse bearing subcutaneous LNCaP (left side of mouse image) or PC-3 (right side of mouse image) tumor xenograft were injected in the tail vein with the labeled antibody or free In111. Images of the same mouse were obtained at the indicated post-injection time.

FIG. 6 shows in vivo therapeutic effect of PSf42.2 illustrated by a graph of the volume of tumor over time. Arrows indicates the day of injection.

FIG. 7 shows stimulation of PSMA internalization by antibody. Cells were biotinylated with thiol-cleavable biotin and then incubated at 37° C. with PSf42.2 (▴) or medium alone (▪). (A) The y-axis illustrates the fraction of internalized PSMA expressed as a percentage of the total cell surface biotinylated PSMA. Data points represent the mean of seven independent experiments. (B) Live LNCaP cells were incubated sequentially with PSf42.2 and goat anti-mouse IgG-Alexa 488 at 4° C., then at 37° C. for the indicated amount of time, and subsequently visualized by epifluorescence microscopy;

FIG. 8 shows a control-corrected sensorgram related to taxol-conjugated PSf42.2 injections over PSMA surfaces;

FIG. 9 shows antibody-mediated cytotoxicity in LNCaP cells. Cells were preincubated for 1 h at room temperature with 2 μg of PSf42.2 or media. (A) cells were cultured in a humidified CO₂ incubator at 37° C. in the absence or presence (white and black bars, respectively) of anti-IgG-saporin for 3 days. The amount of live cells remaining was quantified using crystal violet; (B) cells were cultured in the presence or the indicated concentration of PSf42.2 and anti-IgG-saporin;

FIG. 10 shows a dose-response of anti-PSMA immunoconjugates on prostate cancer cells survival. Three immunoconjugates were constructed by conjugating PSf42.2 to doxorubicin (A), paclitaxel (B) or saporin (C). The graphs show, respectively, a dose-response of antibody drug-conjugate and an equivalent concentration of drug alone on the viability of LNCaP or PC-3 cells.

### DETAILED DESCRIPTION OF THE INVENTION

The present description refers to a number of documents, the content of which is herein incorporated by reference in their entirety.

In order to provide a clear and consistent understanding of the terms used in the present disclosure, a number of definitions are provided below. Moreover, unless defined otherwise, all technical and scientific terms as used herein have the same meaning as commonly understood to one of ordinary skill in the art to which this invention pertains.

As used in the specification and claim(s), the words ‘comprising’ (and any form of comprising, such as ‘comprise’ and ‘comprises’), ‘having’ (and any form of having, such as ‘have’ and ‘has’), ‘including’ (and any form of including, such as ‘include’ and ‘includes’) or ‘containing’ (and any form of containing, such as ‘contain’ and ‘contains’), are inclusive or open-ended and do not exclude additional, unrecited elements or process steps.

The present invention relates in one aspect thereof to isolated antibodies or antigen binding fragments capable of binding to prostate specific membrane antigen (PSMA). More particularly, the present invention relates to diagnostic and/or therapeutic antibodies or antigen binding fragments having specificity for PSMA.

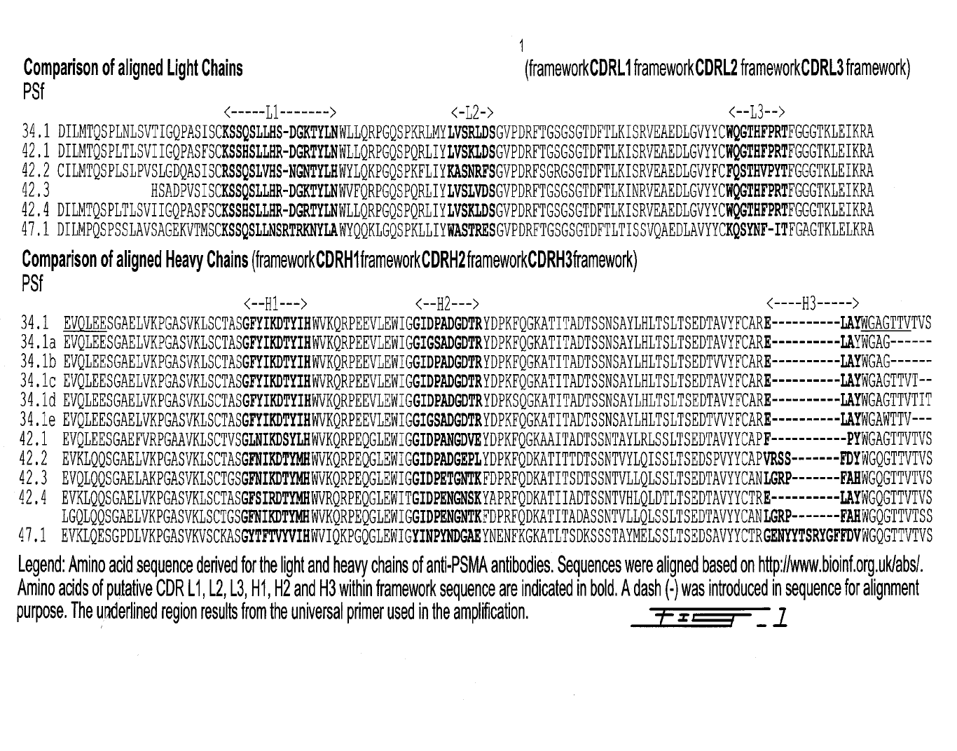

In accordance with the present invention, the antigen-binding fragments may originate from the variable domain of an antibody selected from the group consisting of antibody PSf34.1 (including PSf34.1 as well as PSf34.1a to PSf34.1e), antibody PSf42.1, antibody PSf42.2, antibody PSf42.3, antibody PSf42.4 and antibody PSf47.1. The amino acid sequence of the light chain and heavy chain of each of these antibodies is represented in FIG. 1. A person of skill in the art will know how to identify the antigen binding fragments from these amino acid sequences.

The binding site of an antibody has mainly been attributed to the complementarity-determining regions (CDRs). In some instances, a single CDR (e.g., V_{H} CDR3) may be sufficient to provide antigen recognition and specificity of the antibody. The polypeptide, antibody or antigen-binding fragment of the present invention may preferably comprise the heavy and light chain CDR3s of the antibodies listed in FIG. 1. The polypeptide, antibody or antigen-binding fragment may further comprise the CDR2s of the antibodies listed in FIG. 1. The polypeptide, antibody or antigen-binding fragment may also comprise the CDR1s of the antibodies listed in FIG. 1. The polypeptide, antibody or antigen-binding fragment may further comprise any combinations of the CDRs.

CDRs may be identified by analyzing the amino acid sequence and/or structure of the variable domain of an antibody. Computer-implemented analysis and modeling of antigen-binding site are based on homology analysis comparing the target antibody sequence with those of antibodies with known structures or structural motifs in existing data bases. By using such homology-based modeling methods approximate three-dimensional structure of the target antibody is constructed (Kabat and Wu (1972) Proc. Natl. Acad. Sci. USA 69: 960 964). More recently, the canonical loop concept has been incorporated into the computer-implemented structural modeling of an antibody combining site (Chothia et al. (1989) Nature (London) 342:877; Chothia and Lesk J M B 196:901 (1987)). It is also possible to improve the modeling of CDRs of antibody structures by combining the homology-based modeling with conformational search procedures (Martin, A. C. R. (1989) PNAS 86: 9268-72). Antibody modeling software are also available for determining the CDRs (AbM: Accelrys, Cambridge, U.K.)

The position of the CDRs was determined herein by looking at the amino acid sequence of the variable domain of the light or heavy chain using the following criteria (Xaa is any amino acid).

| | |
|---|---|
| CDR-L1 | |
| Start | Approx residue 24 |
| Residue before | Usually a Cys |
| Residue after | Usually a Trp. Typically Trp-Tyr-Gln, but also, |
| | Trp-Leu-Gln, Trp- Phe-Gln, Trp-Tyr-Leu |
| Length | 10 to 17 residues |
| CDR-L2 | |
| Start | Usually 16 residues after the end of L1 |
| Residues before | generally Ile-Tyr, but also, Val-Tyr, Ile-Lys, Ile-Phe |
| Length | Usually 7 residues |
| CDR-L3 | |
| Start | Usually 33 residues after end of L2 |
| Residue before | Usually Cys |
| Residues after | Usually Phe-Gly-Xaa-Gly |
| Length | 7 to 11 residues |
| CDR-H1 | |
| Start | Approx residue 26 (Usually 4 after a Cys) [Chothia/ |
| | AbM definition]; |
| | Kabat definition starts 5 residues later |
| Residues before | Usually Cys-Xaa-Xaa-Xaa |
| Residues after | Usually a Trp. Typically Trp-Val, but also, Trp-Ile, |
| | Trp-Ala |
| Length | 10 to 12 residues [AbM definition]; |
| | Chothia definition excludes the last 4 residues |
| CDR-H2 | |
| Start | Usually 15 residues after the end of Kabat/AbM |
| | definition) of CDR-H1 |
| Residues before | typically Leu-Glu-Trp-Ile-Gly, but a number of |
| | variations |
| Residues after | Lys/Arg-Leu/Ile/Val/Phe/Thr/Ala-Thr/Ser/Ile/Ala |
| Length | Kabat definition 16 to 19 residues; |
| | AbM (and recent Chothia) definition ends 7 residues |
| | earlier |
| CDR-H3 | |
| Start | Usually 33 residues after end of CDR-H2 (Usually |
| | 2 after a Cys) |
| Residues before | Usually Cys-Xaa-Xaa (typically Cys-Ala-Arg) |
| Residues after | Usually Trp-Gly-Xaa-Gly |
| Length | 3 to 25 residues |

Antibodies and Antigen Binding Fragments that Binds to PSMA

Comparison of the amino acid sequences of the light chain variable domains or the heavy chain variable domains of antibodies showing the greatest characteristics allowed us to derive consensus sequences within the CDRs and within the variable regions. The consensus for CDRs are provided in SEQ ID Nos: 61 to 68.

The variable regions described herein may be fused with constant regions of a desired species thereby allowing recognition of the antibody by effector cells of the desired species. The constant region may originate, for example, from an IgG1, IgG2, IgG3, or IgG4 subtype. In an embodiment of the invention, the constant region may be of human origin. In another embodiment of the invention, the constant region may be of murine origin. Cloning or synthesizing a constant region in frame with a variable region is well within the scope of a person of skill in the art and may be performed, for example, by recombinant DNA technology.

In certain embodiments of the present invention, antibodies that bind to PSMA may be of the IgG1, IgG2, IgG3, or IgG4 subtype. More specific embodiments of the invention relates to an antibody of the IgG1 subtype. In another specific embodiments of the invention relates to an antibody of the IgG2 subtype. In yet another specific embodiments of the invention relates to an antibody of the IgG3 subtype. The antibody may be a humanized antibody of the IgG1 subtype that is biologically active in mediating antibody-dependent cellular cytotoxicity (ADCC), complement-mediated cytotoxicity (CMC), or associated with immune complexes. The typical ADCC involves activation of natural killer (NK) cells and is reliant on the recognition of antibody-coated cells by Fc receptors on the surface of the NK cells. The Fc receptors recognize the Fc domain of antibodies such as is present on IgG1, which bind to the surface of a target cell, in particular a cancerous cell that expresses an antigen, such as PSMA. Once bound to the Fc receptor of IgG the NK cell releases cytokines and cytotoxic granules that enter the target cell and promote cell death by triggering apoptosis.

The present invention described a collection of antibodies that bind to PSMA. In certain embodiments, the antibodies may be selected from the group consisting of polyclonal antibodies, monoclonal antibodies such as chimeric or humanized antibodies, antibody fragments such as antigen binding fragments, single chain antibodies, deimmunized antibodies, human antibodies, recombinant antibodies, domain antibodies, and polypeptides with an antigen binding region.

When only one of the light chain variable domain or the heavy chain variable domain is available, an antibody or antigen-binding fragment may be reconstituted by screening a library of complementary variable domains using methods known in the art (Portolano et al. The Journal of Immunology (1993) 150:880-887, Clarkson et al., Nature (1991) 352:624-628).

The present invention therefore provides in another aspect thereof, an isolated antibody or antigen binding fragment comprising a light chain variable domain having;
a. a CDRL1 sequence selected from the group consisting of SEQ ID NO:1, SEQ ID NO:8, SEQ ID NO:14, SEQ ID NO:20, SEQ ID NO:26 and SEQ ID NO:32;
b. a CDRL2 sequence selected from the group consisting of SEQ ID NO:2, SEQ ID NO:9, SEQ ID NO: 15, SEQ ID NO:21, SEQ ID NO:27 and SEQ ID NO: 33, or;
c. a CDRL3 sequence selected from the group consisting of SEQ ID NO: 3, SEQ ID NO:10, SEQ ID NO:16, SEQ ID NO:22, SEQ ID NO:28 and SEQ ID NO: 34.

In accordance with an embodiment of the invention, the isolated antibody or antigen binding fragment may also comprise a complementary heavy chain variable domain.

In accordance with another embodiment of the invention, the isolated antibody or antigen binding fragment may alternatively comprise a heavy chain variable domain having;
a. a CDRH1 sequence selected from the group consisting of SEQ ID NO:4, SEQ ID NO:11, SEQ ID NO:17, SEQ ID NO:23, SEQ ID NO:29 and SEQ ID NO:35;
b. a CDRH2 sequence selected from the group consisting of SEQ ID NO:5, SEQ ID NO:6, SEQ ID NO:12, SEQ ID NO:18, SEQ ID NO:24, SEQ ID NO:30, SEQ ID NO:36 and SEQ ID NO:70 or;
c. a CDRH3 sequence selected from the group consisting of SEQ ID NO:7, SEQ ID NO:13, SEQ ID NO:19, SEQ ID NO:25, SEQ ID NO:31 and SEQ ID NO:37.

In an exemplary embodiment, the antibody or antigen binding fragment may comprise any individual CDR or a combination of CDR1, CDR2 and/or CDR3 of the light chain variable region. The CDR3 may more particularly be selected. Combination may include for example, CDRL1 and CDRL3; CDRL1 and CDRL2; CDRL2 and CDRL3 and; CDRL1, CDRL2 and CDRL3.

In another exemplary embodiment, the antibody or antigen binding fragment may comprise any individual CDR or a combination of CDR1, CDR2 and/or CDR3 of the heavy chain variable region. The CDR3 may more particularly be selected. Combination may include for example, CDRH1 and CDRH3; CDRH1 and CDRH2; CDRH2 and CDRH3 and; CDRH1, CDRH2 and CDRH3.

In accordance with the present invention, the antibody or antigen binding fragment may comprise at least two CDRs of a CDRL1, a CDRL2 or a CDRL3.

Also in accordance with the present invention, the antibody or antigen binding fragment may comprise one CDRL1, one CDRL2 and one CDRL3.

Further in accordance with the present invention, the antibody or antigen binding fragment may comprise:
a. At least two CDRs of a CDRL1, CDRL2 or CDRL3 and;
b. At least two CDRs of a CDRH1, one CDRH2 or one CDRH3.

An exemplary combination of CDRs may be those which are part of the same variable region illustrated in FIG. 1.

The antibody or antigen binding fragment may more preferably comprise one CDRL1, one CDRL2 and one CDRL3.

The antibody or antigen binding fragment may also more preferably comprise one CDRH1, one CDRH2 and one CDRH3.

The antibody or antigen binding fragment may also more preferably comprise one CDRL1, one CDRL2 and one CDRL3 and one CDRH1, one CDRH2 and one CDRH3.

In another aspect the present invention relates to a polypeptide or an antibody comprising (on a single polypeptide chain or on separate polypeptide chains) at least one complementarity-determining region of a light chain variable domain and at least one complementarity-determining region of a heavy chain variable domain of any one of antibody PSf34.1, antibody PSf42.1, antibody PSf42.2, antibody PSf42.3, antibody PSf42.4 or antibody PSf47.1.

In one embodiment of the invention, the polypeptide or antibody may comprise A—at least two CDRs or more specifically the three CDRs of the light chain variable domain and B—at least two CDRs or more specifically the three CDRs of the heavy chain variable domain.

In another aspect the present invention provides an isolated antibody or antigen binding fragment comprising a heavy chain variable domain having;
a. a CDRH1 sequence selected from the group consisting of SEQ ID NO:4, SEQ ID NO:11, SEQ ID NO:17, SEQ ID NO:23, SEQ ID NO:29 and SEQ ID NO:35;
b. a CDRH2 sequence selected from the group consisting of SEQ ID NO:5, SEQ ID NO:6, SEQ ID NO:12, SEQ ID NO:18, SEQ ID NO:24, SEQ ID NO:30, SEQ ID NO:36 and SEQ ID NO:70 or;
c. a CDRH3 sequence selected from the group consisting of SEQ ID NO:7, SEQ ID NO:13, SEQ ID NO:19, SEQ ID NO:25, SEQ ID NO:31 and SEQ ID NO:37.

In accordance with an embodiment of the invention, the isolated antibody or antigen binding fragment may also comprise a complementary light chain variable domain.

In accordance with the present invention, the antibody or antigen binding fragment may comprise one CDRH1, one CDRH2 or one CDRH3.

In accordance with the present invention, the antibody or antigen binding fragment may also comprise one CDRH1, one CDRH2 and one CDRH3.

It is to be understood herein, that the light chain variable region of the specific combination provided above may be changed for any other light chain variable region. Similarly, the heavy chain variable region of the specific combination provided above may be changed for any other heavy chain variable region.

Although preferred polypeptides or antibodies of the invention are those with CDRs which are 100% identical to those of antibody PSf34.1, antibody PSf42.1, antibody PSf42.2, antibody PSf42.3, antibody PSf42.4 or antibody PSf47.1, the skill artisan will know that variations in the amino acid sequence may be tolerated without loosing binding, specificity and/or affinity.

In a more specific embodiment of the invention, the polypeptide or antibody may comprise a) an amino acid sequence which may be from 95 to 100% identical to SEQ ID NO.:14, b) an amino acid sequence which may be from 95 to 100% identical to SEQ ID NO.:15 and/or c) an amino acid sequence which may be from 95 to 100% identical to SEQ ID NO.:16.

In another specific embodiment of the invention, the polypeptide or antibody may comprise a) an amino acid sequence which may be from 95 to 100% identical to SEQ ID NO.:17, b) an amino acid sequence which may be from 95 to 100% identical to SEQ ID NO.:18 and/or c) an amino acid sequence which may be from 95 to 100% identical to SEQ ID NO.:19.

In an exemplary embodiment of the invention, the polypeptide or antibody may comprise for example, A—a) an amino acid sequence which may be from 95 to 100% identical to SEQ ID NO.:14, b) an amino acid sequence which may be from 95 to 100% identical to SEQ ID NO.:15 and/or c) an amino acid sequence which may be from 95 to 100% identical to SEQ ID NO.:16 and B—a) an amino acid sequence which may be from 95 to 100% identical to SEQ ID NO.:17, b) an amino acid sequence which may be from 95 to 100% identical to SEQ ID NO.:18 and/or c) an amino acid sequence which may be from 95 to 100% identical to SEQ ID NO.:19.

In another exemplary embodiment of the invention, the antibody may comprise for example, A—a) an amino acid sequence which may be from 95 to 100% identical to SEQ ID NO.:14, b) an amino acid sequence which may be from 95 to 100% identical to SEQ ID NO.:15 and c) an amino acid sequence which may be from 95 to 100% identical to SEQ ID NO.:16 and B—a) an amino acid sequence which may be from 95 to 100% identical to SEQ ID NO.:17, b) an amino acid sequence which may be from 95 to 100% identical to SEQ ID NO.:18 and c) an amino acid sequence which may be from 95 to 100% identical to SEQ ID NO.:19.

More particularly in accordance with the present invention, the CDR light chain may each independently comprise zero, one or two amino acid substitutions (conservative or non conservative), insertions, deletions or combination thereof.

More particularly in accordance with the present invention, the CDR heavy chain may each independently comprise zero, one or two amino acid substitutions (conservative or non conservative), insertions, deletions or combination thereof.

Also in accordance with the present invention, each CDRs may be separated by random amino acid sequences, by amino acid sequences obtained from antibody databases or by amino acid sequences which are similar to or at least 75%, at least 76%, at least 77%, at least 78%, at least 79%, at least 80% (and up to 100%) identical to the amino acid framework sequences presented in FIG. 1. Such percent identity determination may exclude the CDR sequence.

The present invention relates in another aspect thereof to polypeptides (single polypeptide chain or a polypeptide complex comprising 2 or more polypeptide chains) or PSMA antibodies that may comprise (on a single polypeptide chain or on separate polypeptide chains) all six complementarity-determining region (CDR) of antibody PSf34.1 (including PSf34.1 as well as PSf34.1a to PSf34.1e), antibody PSf42.1, antibody PSf42.2, antibody PSf42.3, antibody PSf42.4, antibody PSf47.1 or antigen binding portion thereof.

In an embodiment of the invention, the antibody or antigen binding fragment of the present invention may consist essentially of the six CDRs.

Also encompassed by the present invention are polypeptides or antibodies comprising variable chains having at least one conservative amino acid substitution in at least one of the CDRs described herein.

Also encompassed by the present invention are polypeptides or antibodies comprising variable chains having at least one conservative amino acid substitution in at least two of the CDRs.

Also encompassed by the present invention are polypeptides or antibodies comprising variable chains having at least one conservative amino acid substitution in each of the 3 CDRs.

Also encompassed by the present invention are polypeptides or antibodies comprising variable chains having at least two conservative amino acid substitution in at least one of the CDRs and the other CDRs being as illustrated in FIG. 1 or having one or two conservative amino acid substitutions.

In another embodiments one or more of the CDRs of the present invention may comprise an amino acid insertion or deletion without affecting its activity. Such insertion or deletion may be found at one or both of the CDR's extrimity or within the amino acid sequence of the CDR. Of course, combination of insertion, deletion and/or substitution is also contemplated.

In another aspect, the present invention relates to a polypeptide, antibody or antigen binding fragment comprising (on a single polypeptide chain or on separate polypeptide chains) at least one complementarity-determining region of a light chain variable domain and at least one complementarity-determining region of a heavy chain variable domain of one of the antibodies or antigen binding fragment described herein.

The present invention relates in another aspect thereof to antibodies that may comprise (on a single polypeptide chain or on separate polypeptide chains) all six complementarity-determining region (CDR) of the antibody or antigen binding fragment described herein.

The antibodies or antigen binding fragment of the present invention may further comprise additional amino acids flanking the amino and/or carboxy region of the CDR(s). Those additional amino acids may be identical to the framework regions of the corresponding antibodies described herein or may include, for example, conservative amino acid substitution.

The antibodies or antigen binding fragment of the present invention includes those having a CDR sequence encompassed by the consensus CDR sequence formulas described herein.

In accordance with the present invention, the antibody may comprise a CDRL1 sequence comprising or consisting of formula:

(SEQ ID NO: 61: CDRL1 consensus)
X₁ₐSSX₂ₐSLX₃ₐX₄ₐX₅ₐX₆ₐX₇ₐX₈ₐX₉ₐX₁₀ₐYLX₁₁ₐ

wherein X₁ₐ may be for example, a basic amino acid;
wherein X₂ₐ may be for example, glutamine or histidine;
wherein X₃ₐ may be for example, an hydrophobic amino acid;
wherein X₄ₐ may be for example, asparagine or histidine;
wherein X₅ₐ may be for example, serine or arginine;
wherein X₆ₐ may be for example, absent or arginine;
wherein X₇ₐ may be for example, asparagine, arginine or threonine;
wherein X₈ₐ may be for example, glycine or arginine;
wherein X₉ₐ may be for example, a basic amino acid or asparagine;
wherein X₁₀ₐ may be for example, threonine or asparagine and;
wherein X₁₁ₐ may be for example, asparagine, histidine or alanine.

In accordance with the present invention, X₁ₐ may be for example, arginine or lysine. More particularly, X₁ₐ may be for example, lysine.

In accordance with the present invention, X₂ₐ may be for example, glutamine.

In accordance with the present invention, X₃ₐ may be for example, valine or leucine. More particularly, X₃ₐ may be for example leucine.

In accordance with the present invention, X₄ₐ may be for example, histidine.

In accordance with the present invention, X₅ₐ may be for example, serine or arginine.

In accordance with the present invention, X₆ₐ may be for example, arginine.

In accordance with the present invention, X₇ₐ may be for example, aspartic acid, asparagine or threonine. More particularly, X₇ₐ may be for example, aspartic acid.

In accordance with the present invention, X₈ₐ may be for example, glycine.

In accordance with the present invention, X₉ₐ may be for example, arginine, lysine, or asparagine. More particularly, X₉ₐ may be for example, arginine or lysine. Even more particularly, X₉ₐ may be for example, lysine.

In accordance with the present invention, X₁₀ₐ may be for example, threonine.

In accordance with the present invention, X₁₁ₐ may be for example, asparagine.

In accordance with the present invention, the antibody may comprise a CDRL2 sequence comprising or consisting of formula:

(SEQ ID NO: 62: CDRL2 consensus 1)
LVSX_{1b}X_{2b}DX_{3b}

Wherein X_{1b} may be for example, a basic amino acid or leucine;
wherein X_{2b} may be for example, an hydrophobic amino acid, and;
wherein X_{3b} may be for example, serine or absent.

In accordance with the present invention, X_{1b} is arginine or lysine or leucine. More particularly, X_{1b} may be arginine or lysine.

In accordance with the present invention, X_{2b} may be for example, leucine or valine. More particularly, X_{2b} may be for example, leucine.

In accordance with the present invention, X_{3b} may be for example, serine.

In accordance with the present invention, the antibody may comprise a CDRL2 sequence comprising or consisting of formula:

| | |
|---|---|
| X_{1c}ASX_{2c}RX_{3c}S | (SEQ ID NO: 63: CDRL2 consensus 2) |

Wherein X_{1c} may be for example, lysine or trytophan;
wherein X_{2c} may be for example, asparagine and threonine, and;
wherein X_{3c} may be for example, phenylalanine or glutamic acid.

In accordance with the present invention, the antibody may comprise a CDRL3 sequence comprising or consisting of formula:

| | |
|---|---|
| X_{1d}QX_{2d}THX_{3d}PX_{4d}T | (SEQ ID NO: 64: CDRL3 consensus) |

Wherein X_{1d} may be for example, an aromatic amino acid;
wherein X_{2d} may be for example, serine or glycine;
wherein X_{3d} may be for example, phenylalanine or valine, and;
wherein X_{4d} may be for example, arginine or tyrosine.

In accordance with the present invention, X_{1d} may be for example, phenylalanine or tryptophan. More particularly, X_{1d} may be for example, tryptophan.

In accordance with the present invention, X_{2d} may be for example, glycine;

In accordance with the present invention, X_{3d} may be for example, phenylalanine.

In accordance with the present invention, X_{4d} may be for example, arginine.

In accordance with the present invention, the antibody may comprise a CDRH1 sequence comprising or consisting of formula:

(SEQ ID NO: 65: CDRH1 consensus 1)
GX₁ₑX₂ₑX₃ₑX₄ₑX₅ₑX₆ₑX₇ₑX₈ₑH

Wherein X₁ₑ may be for example, an hydrophobic amino acid or tyrosine;
wherein X₂ₑ may be for example, asparagine, serine, tyrosine or threonine;
wherein X₃ₑ may be for example, an hydrophobic amino acid;
wherein X₄ₑ may be for example, a basic amino acid or threonine;
wherein X₅ₑ may be for example, valine or aspartic acid;
wherein X₆ₑ may be for example, an hydrophilic amino acid or tyrosine;
wherein X₇ₑ may be for example, tyrosine or valine, and;
wherein X₈ₑ may be for example, an hydrophobic amino acid.

In accordance with the present invention, X₁ₑ may be for example, phenylalanine, leucine or tyrosine. More particularly, X₁ₑ may be for example, phenylalanine.

In accordance with the present invention, X₂ₑ may be for example, asparagine.

In accordance with the present invention, X₃ₑ may be for example, phenylalanine or isoleucine. More particularly, X₃ₑ may be for example, isoleucine.

In accordance with the present invention, X₄ₑ may be for example, lysine, arginine or threonine. More particularly, X₄ₑ may be for example, lysine.

In accordance with the present invention, X₅ₑ may be for example, aspartic acid.

In accordance with the present invention, X₆ₑ may be for example, threonine, serine or tyrosine. More particularly, X₆ₑ may be for example, threonine.

In accordance with the present invention, X₇ₑ may be for example, tyrosine.

In accordance with the present invention, X₈ₑ may be for example, methionine, isoleucine or leucine.

In accordance with the present invention, the antibody may comprise a CDRH1 sequence comprising or consisting of formula:

(SEQ ID NO: 66: CDRH1 consensus 2)
GX_{1f}X_{2f}IX_{3f}DX_{4f}YX_{5f}H

wherein X_{1f} may be for example, an hydrophobic amino acid;
wherein X_{2f} may be for example, asparagine, serine or tyrosine;
wherein X_{3f} may be for example, a basic amino acid;
wherein X_{4f} may be for example, an hydrophilic amino acid, and;
wherein X_{5f} may be for example, an hydrophobic amino acid.

In accordance with the present invention, X_{1f} may be for example, phenylalanine or leucine. More particularly, X_{1f} may be for example, phenylalanine.

In accordance with the present invention, X_{2f} may be for example, asparagine.

In accordance with the present invention, X_{3f} may be for example, lysine or arginine. More particularly, X_{3f} may be for example, lysine.

In accordance with the present invention, X_{4f} may be for example, serine or threonine. More particularly, X_{4f} may be for example, threonine.

In accordance with the present invention, X_{5f} may be for example, leucine, isoleucine or methionine. More particularly, X_{5f} may be for example, methionine.

In accordance with the present invention, the antibody may comprise a CDRH2 sequence comprising or consisting of formula:

(SEQ ID NO: 67: CDRH2 consensus 1)
GIX_{1g}X_{2g}X_{3g}X_{4g}GX_{5g}X_{6g}X_{7g}

Wherein X_{1g} may be for example, aspartic acid or glycine;
wherein X_{2g} may be for example, proline or serine;
wherein X_{3g} may be for example, alanine or glutamic acid;
wherein X_{4g} may be for example, threonine, asparagine or aspartic acid;
wherein X_{5g} may be for example, aspartic acid, glutamic acid or asparagine;
wherein X_{6g} may be for example, threonine, serine, valine or proline, and;
wherein X_{7g} is a basic amino acid, glutamic acid or leucine.

In accordance with the present invention, X_{1g} may be for example, aspartic acid.

In accordance with the present invention, X_{2g} may be for example, proline.

In accordance with the present invention, X_{3g} may be for example, alanine.

In accordance with the present invention, X_{4g} may be for example, aspartic acid.

In accordance with the present invention, X_{5g} may be for example, aspartic acid.

In accordance with the present invention, X_{6g} may be for example, threonine.

In accordance with the present invention, X_{7g} is lysine, arginine, glutamic acid or leucine. More particularly, X_{7g} is lysine or arginine.

In accordance with the present invention, the antibody may comprise a CDRH2 sequence comprising or consisting of formula:

(SEQ ID NO: 68: CDRH2 consensus 2)
GIDPEX₁ₕGNX₂ₕK

Wherein X₁ₕ may be for example, threonine or arginine
wherein X₂ₕ may be for example, a neutral hydrophilic amino acid (threonine or serine).

The framework region of the heavy and/or light chains described herein may be derived from one or more of the framework regions illustrated herein. The antibody or antigen binding fragments may thus comprise one or more of the CDRs described herein (e.g., selected from the specific CDRs of SEQ ID NO:1 to 37 or consensus CDRs of SEQ ID NO:61 to 68) and framework regions originating from those illustrated herein wherein such framework region share at least 75%, at least 76%, at least 77%, at least 78%, at least 79%, at least 80% (and up to 100%) identity to the amino acid framework sequences presented in FIG. 1. In FIG. 1, the expected CDRs are shown in bold, while the framework regions are not.

The framework region of the light chain described herein (as antibodies or fragments, kits, methods, uses etc.) may have at least 67, 68, 69, etc. amino acids of SEQ ID NO:38, at least least 67, 68, 69, etc. amino acids of SEQ ID NO:45, least 70, 71, 72, etc. amino acids of SEQ ID NO:47, at least 66, 67, 68, etc. amino acids of SEQ ID NO:49, at least 65, 66, 67, etc. amino acids of SEQ ID NO:51 or at least 66, 67, 68, etc. amino acids of SEQ ID NO:53.

The framework region of the heavy chain described herein (as antibodies or fragments, kits, methods, uses etc.) may have at least 71, 72, 73 etc. amino acids of SEQ ID NO: 39, at least 66, 67, 68 etc. amino acids of SEQ ID NO: 40, at least 67, 68, 69 etc. amino acids of SEQ ID NO: 46, at least 63, 64, 65 etc. amino acids of SEQ ID NO: 48, at least 70, 71, 71 etc. amino acids of SEQ ID NO: 50, at least 72, 73, 74 etc. amino acids of SEQ ID NO: 52, at least 71, 72, 73 etc. amino acids of SEQ ID NO: 54 or at least 68, 69, 70 etc. amino acids of SEQ ID NO: 69.

The framework region of the heavy chain may have at the 5′ end, an amino acid sequence comprising amino acids 1 to 17, 2 to 17, 3 to 17, 4 to 17, 5 to 17, 6 to 17, 7 to 17, 8 to 17, 9 to 17, 10 to 17, 11 to 17, 12 to 17, 13 to 17, 14 to 17, 15 to 17, 16 to 17 or amino acid 17 of any one of SEQ ID NO:39, SEQ ID NO: 40, SEQ ID NO:41, SEQ ID NO:42, SEQ ID NO:43, SEQ ID NO:44, SEQ ID NO:46, SEQ ID NO:48, SEQ ID NO:50, SEQ ID NO:52, SEQ ID NO:54 or SEQ ID NO:69.

The framework region of the light chain may have at the 5′ end, an amino acid sequence comprising amino acids 1 to 13, 2 to 13, 3 to 13, 4 to 13, 5 to 13, 6 to 13, 7 to 13, 8 to 13, 9 to 13, 10 to 13, 11 to 13, 12 to 13, or amino acid 13 of any one of SEQ ID NO: 38, SEQ ID NO:45, SEQ ID NO:47, SEQ ID NO:51 or SEQ ID NO:53.

Also encompassed by the present invention are antibodies comprising a light chain comprising one of the variable region illustrated in FIG. 1 and a heavy chain comprising one of the variable region illustrated in FIG. 1. The light chain and heavy chain may comprise a constant domain. Combinations of light chains and heavy chains of FIG. 1 are also encompassed by the present invention.

Antibodies or antigen binding fragments that contain the light chain and heavy chain variable regions are also provided in the present invention. Additionally, certain embodiments include antigen binding fragments, variants, and derivatives of these light and heavy chain variable regions.

Yet other exemplary embodiments of the invention includes an isolated antibody or antigen binding fragment capable of specific binding to PSMA or to a variant thereof, the antibody comprising:
a. the light chain variable domain defined in SEQ ID NO.:38 and the heavy chain variable domain defined in SEQ ID NO.:39,
b. the light chain variable domain defined in SEQ ID NO.:38 and the heavy chain variable domain defined in SEQ ID NO.:40;
c. the light chain variable domain defined in SEQ ID NO.:38 and the heavy chain variable domain defined in SEQ ID NO.:41;
d. the light chain variable domain defined in SEQ ID NO.:38 and the heavy chain variable domain defined in SEQ ID NO.:42,
e. the light chain variable domain defined in SEQ ID NO.:38 and the heavy chain variable domain defined in SEQ ID NO.:43,
f. the light chain variable domain defined in SEQ ID NO.:38 and the heavy chain variable domain defined in SEQ ID NO.:44,
g. the light chain variable domain defined in SEQ ID NO.:45 and the heavy chain variable domain defined in SEQ ID NO.:46,
h. the light chain variable domain defined in SEQ ID NO.:47 and the heavy chain variable domain defined in SEQ ID NO.:48,
i. the light chain variable domain defined in SEQ ID NO.:49 and the heavy chain variable domain defined in SEQ ID NO.:50,
j. the light chain variable domain defined in SEQ ID NO.:49 and the heavy chain variable domain defined in SEQ ID NO.:69;
k. the light chain variable domain defined in SEQ ID NO.:51 and the heavy chain variable domain defined in SEQ ID NO.:52,
l. the light chain variable domain defined in SEQ ID NO.:53 and the heavy chain variable domain defined in SEQ ID NO.:54, or;
m. the light chain and heavy chain combination described in any one of a. to l. above and further comprising zero or at least one, at least two, at least three or at least four amino acid substitutions (conservative or non conservative), insertion, deletion or combination thereof in one, two, three, four, five or six of the CDRs and wherein one or both of the framework region (i.e., of the light chain and/or heavy chain variable region) has at least 75%, at least 76%, at least 77%, at least 78%, at least 79%, at least 80% (and up to 100%) identity with the amino acid framework sequences presented in FIG. 1.

In accordance with the present invention, the substitution, insertion or deletion may be located preferably within the framework region. Alternatively, the substitution, insertion or deletion may be located within the one or more of the CDRs.

The present invention thus encompasses an antibody or antigen binding fragment having at least one amino acid substitution, insertion or deletion in the framework region and at least one amino acid substitution, insertion or deletion in one or more of the CDRs.

In accordance with an embodiment of the invention, the light chain framework region may be at least 80% identical (and up to 100% identical) to the amino acid framework sequences presented in FIG. 1.

In accordance with an embodiment of the invention, the heavy chain framework region may be at least 75%, at least 76%, at least 77%, at least 78%, at least 79%, at least 80% (and up to 100%) identical to the amino acid framework sequences presented in FIG. 1.

More particularly, the heavy chain framework region may be at least 79%, at least 80%, at least 81% identical to the amino acid framework sequences presented in FIG. 1.

In another embodiment of the invention, the light chain framework region may be at least 80% (and up to 100%) identical to the amino acid framework sequences presented in FIG. 1 and the heavy chain framework region may be at least 75%, at least 76%, at least 77%, at least 78%, at least 79%, at least 80% (and up to 100%) identical to the amino acid framework sequences presented in FIG. 1.

In yet another embodiment, the light chain framework region may be at least 80% identical to the amino acid framework sequences presented in FIG. 1 and the heavy chain framework region may be at least 80% identical to the amino acid framework sequences presented in FIG. 1.

In an aspect of the invention, the CDRs (of the heavy chain and/or the light chain) may each independently comprises zero, one or two amino acid substitution, deletion or insertion. More particularly, the CDRs (of the heavy chain and/or the light chain) may each independently comprises zero or one amino acid substitution, deletion or insertion. Even more particularly, the CDRs (of the heavy chain and/or the light chain) may be identical to those of FIG. 1.

In a further aspect of the invention, the CDRs of the heavy chain may each independently comprises zero, one or two amino acid substitution, deletion or insertion. More particularly, the CDRs of the heavy chain may each independently comprises zero or one amino acid substitution, deletion or insertion. Even more particularly, the CDRs of the heavy chain may be identical to those of FIG. 1. As indicated above and in an embodiment of the invention, the CDRs of the light chain may each independently comprises zero, one, two, three of four amino acid substitution, deletion or insertion in comparison with the corresponding CDRs of FIG. 1.

In a further aspect of the invention, the CDRs of the light chain may each independently comprises zero, one or two amino acid substitution, deletion or insertion. More particularly, the CDRs of the light chain may each independently comprises zero or one amino acid substitution, deletion or insertion. Even more particularly, the CDRs of the light chain may be identical to those of FIG. 1. As indicated above and in an embodiment of the invention, the CDRs of the heavy chain may each independently comprises zero, one, two, three of four amino acid substitution, deletion or insertion in comparison with the corresponding CDRs of FIG. 1.

In an exemplary embodiment of the invention, the polypeptide or antibody may comprise an amino acid sequence which may be from 80 to 100% (including any individual percentage therebetween), 90 to 100%, or 95 to 100% (98% to 100%, 98.5% to 100%; 99% to 100%) identical to any one of SEQ ID NO.:1 to SEQ ID NO.:37.

As such, the variable regions that are contained in the anti-PSMA antibodies or antigen binding fragments may be have 80%, 85%, 90%, 91%, 92%, 93%, 94%, 95%, 96%, 97%, 98%, 99% or 100% sequence identity to the variable regions presented in FIG. 1. Those skilled in the art will also recognize that the variants may include conservative amino acid changes, amino acid substitutions, deletions, or additions in the variable regions listed in FIG. 1.

Also, the CDRs that are contained in the anti-PSMA antibodies or antigen binding fragments may be variant CDRs with 80%, 85%, 90%, 91%, 92%, 93%, 94%, 95%, 96%, 97%, 98%, 99% or 100% sequence identity to the CDR sequences presented in FIG. 1. Those skilled in the art will also recognize that the variants may include conservative amino acid changes, amino acid substitutions, deletions, or additions in the CDR sequences listed in FIG. 1.

Other exemplary embodiments of the invention includes an isolated antibody or antigen binding fragment capable of specific binding to PSMA or to a variant thereof, the antibody comprising:
a. the 3CDRs of a light chain variable domain defined in SEQ ID NO:38 and the 3CDRs of a heavy chain variable domain defined in either SEQ ID NO:39, SEQ ID NO:40, SEQ ID NO:41, SEQ ID NO:42, SEQ ID NO:43 or SEQ ID NO:44,
b. the 3CDRs of a light chain variable domain defined in SEQ ID NO.:45 and the 3CDRs of a heavy chain variable domain defined in SEQ ID NO.:46;
c. the 3CDRs of a light chain variable domain defined in SEQ ID NO.:47 and the 3CDRs of a heavy chain variable domain defined in SEQ ID NO.:48;
d. the 3CDRs of a light chain variable domain defined in SEQ ID NO.:49 and the 3CDRs of a heavy chain variable domain defined in SEQ ID NO.:50;
e. the 3CDRs of a light chain variable domain defined in SEQ ID NO.:49 and the 3CDRs of a heavy chain variable domain defined in SEQ ID NO.:70
f. the 3CDRs of a light chain variable domain defined in SEQ ID NO.:51 and the 3CDRs of a heavy chain variable domain defined in SEQ ID NO.:52; or
g. the 3CDRs of a light chain variable domain defined in SEQ ID NO.:53 and the 3CDRs of a heavy chain variable domain defined in SEQ ID NO.:54.

Again variations in the corresponding framework region of FIG. 1, such as mentioned elsewhere herein, are encompassed by the present invention.

Again, the light chain variable region of the specific combination provided above may be changed for any other light chain variable region described herein. Similarly, the heavy chain variable region of the specific combination provided above may be changed for any other heavy chain variable region described herein.

Variant Antibody and Antigen Binding Fragments

As indicated above, the present invention also encompasses variants of the antibodies or antigen binding fragments described herein. Variant antibodies or antigen binding fragments included are those having a variation in the amino acide sequence. For example, variant antibodies or antigen binding fragments included are those having at least one variant CDR (two, three, four, five or six variant CDRs), a variant light chain variable domain, a variant heavy chain variable domain, a variant light chain and/or a variant heavy chain. Variant antibodies or antigen binding fragments included in the present invention are those having, for example, similar or improved binding affinity in comparison with the original antibody or antigen binding fragment.

As used herein the term “variant” applies to any of the sequence described herein and includes for example, a variant CDR (either CDRL1, CDRL2, CDRL3, CDRH1, CDRH2 and/or CDRH3), a variant light chain variable domain, a variant heavy chain variable domain, a variant light chain, a variant heavy chain, a variant antibody, and a variant antigen binding fragment.

Variant antibodies or antigen binding fragments encompassed by the present invention are those which may comprise an insertion, a deletion or an amino acid substitution (conservative or non-conservative). These variants may have at least one amino acid residue in its amino acid sequence removed and a different residue inserted in its place.

The sites of greatest interest for substitutional mutagenesis include the hypervariable regions (CDRs), but modifications in the framework region or even in the constant region are also contemplated. Conservative substitutions may be made by exchanging an amino acid (of a CDR, variable chain, antibody, etc.) from one of the groups listed below (group 1 to 6) for another amino acid of the same group.

Other exemplary embodiment of conservative substitutions are shown in Table 1A under the heading of “preferred substitutions”. If such substitutions result in a undesired property, then more substantial changes, denominated “exemplary substitutions” in Table 1A, or as further described below in reference to amino acid classes, may be introduced and the products screened.

It is known in the art that variants may be generated by substitutional mutagenesis and retain the biological activity of the polypeptides of the present invention. These variants have at least one amino acid residue in the amino acid sequence removed and a different residue inserted in its place. For example, one site of interest for substitutional mutagenesis may include a site in which particular residues obtained from various species are identical. Examples of substitutions identified as “conservative substitutions” are shown in Table 1A. If such substitutions result in a change not desired, then other type of substitutions, denominated “exemplary substitutions” in Table 1A, or as further described herein in reference to amino acid classes, are introduced and the products screened.

As is known in the art, it may be of interest to modify the biological activity of a polypeptide by amino acid substitution, insertion or deletion. For example, modification of a polypeptide may result in an increase in the polypeptide's biological activity, may modulate its toxicity, may result in changes in bioavailability or in stability, or may modulate its immunological activity or immunological identity.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(group 1) hydrophobic (aliphatic): norleucine, methionine (Met), Alanine (Ala), Valine (Val), Leucine (Leu), Isoleucine (Ile)
(group 2) neutral hydrophilic: Cysteine (Cys), Serine (Ser), Threonine (Thr)
(group 3) acidic: Aspartic acid (Asp), Glutamic acid (Glu)
(group 4) basic: Asparagine (Asn), Glutamine (Gln), Histidine (His), Lysine (Lys), Arginine (Arg)
(group 5) residues that influence chain orientation: Glycine (Gly), Proline (Pro); and
(group 6) aromatic: Tryptophan (Trp), Tyrosine (Tyr), Phenylalanine (Phe)

Non-conservative substitutions will entail exchanging a member of one of these classes for another.

Thus, in some cases, the basic amino acids Lys, Arg and His may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, Ile, and Leu are interchangeable but because of size Gly and Ala are more closely related and Val, Ile and Leu are more closely related to each other, and the aromatic amino acids Phe, Trp and Tyr may be interchangeable. It should be further noted that if the polypeptides are made synthetically, substitutions by amino acids, which are not naturally encoded by DNA (non-naturally occurring or unnatural amino acid) may also be made. A non-naturally occurring amino acid is to be understood herein as an amino acid which is not naturally produced or found in a mammal. A non-naturally occurring amino acid comprises a D-amino acid, an amino acid having an acetylaminomethyl group attached to a sulfur atom of a cysteine, a pegylated amino acid, etc. The inclusion of a non-naturally occurring amino acid in a defined polypeptide sequence will therefore generate a derivative of the original polypeptide.

TABLE 1A
Amino acid substitution

| | | Exemplary |
|---|---|---|
| Original | | conservative |
| residue | Exemplary substitution | substitution |
| Ala (A) | Val, Leu, Ile, Gly, Ser | Val |
| Arg (R) | Lys, Gln, Asn | Lys |
| Asn (N) | Gln, His, Lys, Arg, Asp | Gln |
| Asp (D) | Glu, Asn | Glu |
| Cys (C) | Ser, Ala | Ser |
| Gln (Q) | Asn; Glu | Asn |
| Glu (E) | Asp, Gln | Asp |
| Gly (G) | Ala, Pro | Ala |
| His (H) | Asn, Gln, Lys, Arg, | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile, Tyr | Leu |
| Phe (F) | Met, Leu, Val, Ile, Ala, Tyr | Tyr, Leu |
| Pro (P) | Ala, Gly | Ala, Gly |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | norleucine | |

Polypeptides of the present invention may comprise for example, those containing amino acid sequences modified either by natural processes, such as posttranslational processing or by chemical modification techniques which are known in the art. Modifications may occur anywhere in a polypeptide including the polypeptide backbone, the amino acid side chains and the amino- or carboxy-terminus. A given polypeptide may contain many types of modifications. It is to be understood herein that more than one modification to the polypeptides described herein are encompassed by the present invention to the extent that the biological activity is substantially similar to the original polypeptide. Polypeptide modification may comprise, for example, amino acid insertion, deletion and substitution (i.e., replacement), either conservative or non-conservative (e.g., D-amino acids) in the polypeptide sequence where such changes do not substantially alter the overall biological activity of the polypeptide.

Variation in the amino acid sequence of the variant antibody or antigen binding fragment thus may include an amino acid addition, deletion, insertion, substitution etc., one or more modification in the backbone or side-chain of one or more amino acid, or an addition of a group or another molecule to one or more amino acids (side-chains or backbone).

Variant antibody or antigen binding fragment may have substantial sequence similarity and/or sequence identity in its amino acid sequence in comparison with that the original antibody or antigen binding fragment amino acid sequence. The degree of similarity between two sequences is based upon the percentage of identities (identical amino acids) and of conservative substitution.

In addition, a non-naturally occurring amino acid may substitute for a naturally occurring amino acid (i.e., non-naturally occurring conservative amino acid substitution or a non-naturally occurring non-conservative amino acid substitution).

Generally, the degree of similarity and identity between variable chains has been determined herein using the Blast2 sequence program (Tatiana A. Tatusova, Thomas L. Madden (1999), “Blast 2 sequences—a new tool for comparing protein and nucleotide sequences”, FEMS Microbiol Lett. 174:247-250) using default settings, i.e., blastp program, BLOSUM62 matrix (open gap 11 and extension gap penalty 1; gapx dropoff 50, expect 10.0, word size 3) and activated filters.

Percent identity will therefore be indicative of amino acids which are identical in comparison with the original peptide and which may occupy the same or similar position.

Upon calculating the % identity for the framework region, the CDRs have been excluded.

Percent similarity will be indicative of amino acids which are identical and those which are replaced with conservative amino acid substitution in comparison with the original peptide at the same or similar position.

Variants of the present invention therefore comprise those which may have at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity with an original sequence or a portion of an original sequence. As will be understood, the term “at least 80%” includes every listed percentage comprised between 80% and 100% and including 80% and 100%. Unless otherwise specified, similar expression are also to be understood in a similar manner. For example “at least 69%” includes every listed percentage comprised between 69% and 100% and including 69% and 100%.

Exemplary embodiments of variants are those having at least 81% sequence identity to a sequence described herein and 81%, 82%, 83%, 84%, 85%, 86%, 87%, 88%, 89%, 90%, 91%, 92%, 93%, 94%, 95%, 96%, 97%, 98%, 99% or 100% sequence similarity with an original sequence or a portion of an original sequence.

Other exemplary embodiments of variants are those having at least 82% sequence identity to a sequence described herein and 82%, 83%, 84%, 85%, 86%, 87%, 88%, 89%, 90%, 91%, 92%, 93%, 94%, 95%, 96%, 97%, 98%, 99% or 100% sequence similarity with an original sequence or a portion of an original sequence.

Further exemplary embodiments of variants are those having at least 85% sequence identity to a sequence described herein and 85%, 86%, 87%, 88%, 89%, 90%, 91%, 92%, 93%, 94%, 95%, 96%, 97%, 98%, 99% or 100% sequence similarity with an original sequence or a portion of an original sequence.

Other exemplary embodiments of variants are those having at least 90% sequence identity to a sequence described herein and 90%, 91%, 92%, 93%, 94%, 95%, 96%, 97%, 98%, 99% or 100% sequence similarity with an original sequence or a portion of an original sequence.

Additional exemplary embodiments of variants are those having at least 95% sequence identity to a sequence described herein and 95%, 96%, 97%, 98%, 99% or 100% sequence similarity with an original sequence or a portion of an original sequence.

Yet additional exemplary embodiments of variants are those having at least 97% sequence identity to a sequence described herein and 97%, 98%, 99% or 100% sequence similarity with an original sequence or a portion of an original sequence.

For a purpose of concision the applicant provides herein a Table 1B illustrating exemplary embodiments of individual variants encompassed by the present invention and comprising the specified % sequence identity and % sequence similarity. Each “X” is to be construed as defining a given variant.

TABLE 1B

| | | Percent (%) sequence identity | | | | | | | | | | | | | | | | | | | | |
|---|---|---|---|---|---|---|---|---|---|---|---|---|---|---|---|---|---|---|---|---|---|---|
| | | 80 | 81 | 82 | 83 | 84 | 85 | 86 | 87 | 88 | 89 | 90 | 91 | 92 | 93 | 94 | 95 | 96 | 97 | 98 | 99 | 100 |
| Percent (%) | 80 | X | | | | | | | | | | | | | | | | | | | | |
| sequence similarity | 81 | X | X | | | | | | | | | | | | | | | | | | | |
| | 82 | X | X | X | | | | | | | | | | | | | | | | | | |
| | 83 | X | X | X | X | | | | | | | | | | | | | | | | | |
| | 84 | X | X | X | X | X | | | | | | | | | | | | | | | | |
| | 85 | X | X | X | X | X | X | | | | | | | | | | | | | | | |
| | 86 | X | X | X | X | X | X | X | | | | | | | | | | | | | | |
| | 87 | X | X | X | X | X | X | X | X | | | | | | | | | | | | | |
| | 88 | X | X | X | X | X | X | X | X | X | | | | | | | | | | | | |
| | 89 | X | X | X | X | X | X | X | X | X | X | | | | | | | | | | | |
| | 90 | X | X | X | X | X | X | X | X | X | X | X | | | | | | | | | | |
| | 91 | X | X | X | X | X | X | X | X | X | X | X | X | | | | | | | | | |
| | 92 | X | X | X | X | X | X | X | X | X | X | X | X | X | | | | | | | | |
| | 93 | X | X | X | X | X | X | X | X | X | X | X | X | X | X | | | | | | | |
| | 94 | X | X | X | X | X | X | X | X | X | X | X | X | X | X | X | | | | | | |
| | 95 | X | X | X | X | X | X | X | X | X | X | X | X | X | X | X | X | | | | | |
| | 96 | X | X | X | X | X | X | X | X | X | X | X | X | X | X | X | X | X | | | | |
| | 97 | X | X | X | X | X | X | X | X | X | X | X | X | X | X | X | X | X | X | | | |
| | 98 | X | X | X | X | X | X | X | X | X | X | X | X | X | X | X | X | X | X | X | | |
| | 99 | X | X | X | X | X | X | X | X | X | X | X | X | X | X | X | X | X | X | X | X | |
| | 100 | X | X | X | X | X | X | X | X | X | X | X | X | X | X | X | X | X | X | X | X | X |

The present invention encompasses CDRs, light chain variable domains, heavy chain variable domains, light chains, heavy chains, antibodies and/or antigen binding fragments which comprise at least 80% identity with the sequence described herein.

In an exemplary embodiment of the invention, the antibody may comprise for example, an amino acid sequence which may be at least from 95% to 100% identical to any one of SEQ ID NO.:38 to 54.

In another exemplary embodiment of the invention, the antibody may comprise for example, a fragment of 3 to 30 amino acids (e.g., 3 to 25, 3 to 10) amino acids which is at least from 95% to 100% identical to any one of SEQ ID NO.:38 to 54.

In a specific exemplary embodiment of the invention, the antibody may comprise A—a light chain variable domain which is at least from 95% to 100% identical to SEQ ID NO.:47 and B—a heavy chain variable domain which is at least from 95% to 100% identical to SEQ ID NO.:48.

Production of the Antibodies in Cells

The antibodies that are disclosed herein can be made by a variety of methods familiar to those skilled in the art, such as hybridoma methodology or by recombinant DNA methods.

In another aspect, the present invention thus relates to an isolated cell that may produce the antibody or antigen binding fragment described herein. In accordance with the present invention, the isolated cell may be a hydridoma cell producing an antibody described herein. Alternatively, the isolated cell may be a hydridoma cell producing an antibody having the same epitope specificity as the antibody or antigen binding fragment described herein.

The present invention, therefore encompasses a cell (an isolated cell) which comprises and/or expresses an antibody or antigen binding fragment of the present invention or a portion thereof (e.g., such as during cloning procedures etc.). Although conventional hybridoma cells are contemplated, a person of skill in the art will readily know that other cells are suitable for expressing antibodies or antigen binding fragments, such as bacterial cells, yeast cells, mammalian expression system (e.g., CHO, 293 etc.). Cells that are particularly useful for expression of antibodies, are those which are able to suitably express the antibody (complete antibody, antibody chain(s) or fragments), suitably glycosylate it and/or suitably secrete it.

In an exemplary embodiment of the invention, the antibodies may be produced by the conventional hybridoma technology, where a mouse is immunized with an antigen, spleen cells isolated and fused with myeloma cells lacking HGPRT expression and hybrid cells selected by hypoxanthine, aminopterin and thymine (HAT) containing media.

In an additional exemplary embodiment of the invention, the antibodies may be produced by recombinant DNA methods.

In order to express the antibodies, nucleotide sequences able to encode any one of a light and heavy immunoglobulin chains described herein may be inserted into an expression vector, i.e., a vector that contains the elements for transcriptional and translational control of the inserted coding sequence in a particular host. These elements may include regulatory sequences, such as enhancers, constitutive and inducible promoters, and 5′ and 3′ un-translated regions. Methods that are well known to those skilled in the art may be used to construct such expression vectors. These methods include in vitro recombinant DNA techniques, synthetic techniques, and in vivo genetic recombination.

A variety of expression vector/host cell systems known to those of skill in the art may be utilized to express a polypeptide or RNA derived from nucleotide sequences able to encode any one of a light and heavy immunoglobulin chains described herein. These include, but are not limited to, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; or animal cell systems. For long-term production of recombinant proteins in mammalian systems, stable expression in cell lines may be effected. For example, nucleotide sequences able to encode any one of a light and heavy immunoglobulin chains described herein may be transformed into cell lines using expression vectors that may contain viral origins of replication and/or endogenous expression elements and a selectable or visible marker gene on the same or on a separate vector. The invention is not to be limited by the vector or host cell employed. In certain embodiments of the present invention, the nucleotide sequences able to encode any one of a light and heavy immunoglobulin chains described herein may each be ligated into a separate expression vector and each chain expressed separately. In another embodiment, both the light and heavy chains able to encode any one of a light and heavy immunoglobulin chains described herein may be ligated into a single expression vector and expressed simultaneously.

Alternatively, RNA and/or polypeptide may be expressed from a vector comprising nucleotide sequences able to encode any one of a light and heavy immunoglobulin chains described herein using an in vitro transcription system or a coupled in vitro transcription/translation system respectively.

The term “vector” encompasses, without being limited to, autonomously replicating DNA or RNA molecule into which foreign DNA or RNA fragments may be inserted and then propagated in a host cell for expression and/or amplification of the foreign DNA or RNA molecule. A vector may comprise, without limitation, a linear plasmid and/or circular plasmid.

In general, host cells that contain nucleotide sequences able to encode any one of a light and heavy immunoglobulin chains described herein and/or that express a polypeptide encoded by the nucleotide sequences able to encode any one of a light and heavy immunoglobulin chains described herein, or a portion thereof, may be identified by a variety of procedures known to those of skill in the art. These procedures include, but are not limited to, DNA/DNA or DNA/RNA hybridizations, PCR amplification, and protein bioassay or immunoassay techniques that include membrane, solution, or chip based technologies for the detection and/or quantification of nucleic acid or amino acid sequences. Immunological methods for detecting and measuring the expression of polypeptides using either specific polyclonal or monoclonal antibodies are known in the art. Examples of such techniques include enzyme-linked immunosorbent assays (ELISAs), radioimmunoassays (RIAs), and fluorescence activated cell sorting (FACS). Those of skill in the art may readily adapt these methodologies to the present invention.

Host cells comprising nucleotide sequences able to encode any one of a light and heavy immunoglobulin chains described herein may thus be cultured under conditions for the transcription of the corresponding RNA (mRNA, siRNA, shRNA etc.) and/or the expression of the polypeptide from cell culture. The polypeptide produced by a cell may be secreted or may be retained intracellularly depending on the sequence and/or the vector used. In an exemplary embodiment, expression vectors containing nucleotide sequences able to encode any one of a light and heavy immunoglobulin chains described herein may be designed to contain signal sequences that direct secretion of the polypeptide through a prokaryotic or eukaryotic cell membrane.

Due to the inherent degeneracy of the genetic code, other DNA sequences that encode the same, substantially the same or a functionally equivalent amino acid sequence may be produced and used, for example, to express a polypeptide encoded by nucleotide sequences able to encode any one of a light and heavy immunoglobulin chains described herein. The nucleotide sequences of the present invention may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In addition, a host cell strain may be chosen for its ability to modulate expression of the inserted sequences or to process the expressed polypeptide in the desired fashion. Such modifications of the polypeptide include, but are not limited to, acetylation, carboxylation, glycosylation, phosphorylation, lipidation, and acylation. In an exemplary embodiment, antibodies that contain particular glycosylation structures or patterns may be desired. Post-translational processing, which cleaves a “prepro” form of the polypeptide, may also be used to specify protein targeting, folding, and/or activity. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of the expressed polypeptide.

Those of skill in the art will readily appreciate that natural, modified, or recombinant nucleic acid sequences may be ligated to a heterologous sequence resulting in translation of a fusion polypeptide containing heterologous polypeptide moieties in any of the aforementioned host systems. Such heterologous polypeptide moieties may facilitate purification of fusion polypeptides using commercially available affinity matrices. Such moieties include, but are not limited to, glutathione S-transferase (GST), maltose binding protein, thioredoxin, calmodulin binding peptide, 6-His (His), FLAG, c-myc, hemaglutinin (HA), and antibody epitopes such as monoclonal antibody epitopes.

In yet a further aspect, the present invention relates to a polynucleotide which may comprise a nucleotide sequence encoding a fusion protein. The fusion protein may comprise a fusion partner (e.g., HA, Fc, etc.) fused to the polypeptide (e.g., complete light chain, complete heavy chain, variable regions, CDRs etc.) described herein.

Those of skill in the art will also readily recognize that the nucleic acid and polypeptide sequences may be synthesized, in whole or in part, using chemical or enzymatic methods well known in the art. For example, peptide synthesis may be performed using various solid-phase techniques and machines such as the ABI 431A Peptide synthesizer (PE Biosystems) may be used to automate synthesis. If desired, the amino acid sequence may be altered during synthesis and/or combined with sequences from other proteins to produce a variant protein.

Antibody Conjugates

The antibody or antigen binding fragment of the present invention may be conjugated with a detectable moiety (i.e., for detection or diagnostic purposes) or with a therapeutic moiety (for therapeutic purposes)

A “detectable moiety” is a moiety detectable by spectroscopic, photochemical, biochemical, immunochemical, chemical and/or other physical means. A detectable moiety may be coupled either directly and/or indirectly (for example via a linkage, such as, without limitation, linked with DOTA) to antibodies and antigen binding fragments thereof of the present invention using methods well known in the art. A wide variety of detectable moieties may be used, with the choice depending on the sensitivity required, ease of conjugation, stability requirements and available instrumentation. A suitable detectable moiety include, but is not limited to, a fluorescent label, a radioactive label (for example, without limitation, ¹²⁵I, In¹¹¹, Tc⁹⁹, I¹³¹ and including positron emitting isotopes for PET scanner etc), a nuclear magnetic resonance active label, a luminiscent label, a chemiluminescent label, a chromophore label, an enzyme label (for example and without limitation horseradish peroxidase, alkaline phosphatase, etc.), quantum dots and/or a nanoparticle. Detectable moiety may cause and/or produce a detectable signal thereby allowing for a signal from the detectable moiety to be detected.

In another exemplary embodiment of the invention, the antibody or antigen binding fragment thereof may be coupled (modified) with a therapeutic moiety (e.g., drug (e.g., an anticancer drug), cytotoxic moiety).

In an exemplary embodiment, the antibodies and antigen binding fragments may comprise a chemotherapeutic or cytotoxic agent. For example, the antibody and antigen binding fragments may be conjugated to the chemotherapeutic or cytotoxic agent. Such chemotherapeutic or cytotoxic agents include, but are not limited to, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹³), copper (e.g., Cu⁶⁷)). In other instances, the chemotherapeutic or cytotoxic agent may be comprised of, among others known to those skilled in the art, 5-fluorouracil, adriamycin, irinotecan, taxanes, pseudomonas endotoxin, ricin and other toxins.

Alternatively, in order to carry out the methods of the present invention and as known in the art, the antibody or antigen binding fragment of the present invention (conjugated or not) may be used in combination with a second molecule (e.g., a secondary antibody, etc.) which is able to specifically bind to the antibody or antigen binding fragment of the present invention and which may carry a desirable detectable, diagnostic or therapeutic moiety.

Pharmaceutical Compositions of the Antibodies and their Use

Pharmaceutical compositions of the antibodies and antigen binding fragment are also encompassed by the present invention. The pharmaceutical composition may thus comprise an antibody or an antigen binding fragment and may also contain a pharmaceutically acceptable carrier.

In order to inhibit the growth of a tumor cell or in order to promote tumor cell death, the pharmaceutical composition may comprise a naked antibody or an antigen binding fragment and may also contain a pharmaceutically acceptable carrier. Of course, as indicated herein, it may be useful to also add a therapeutic moiety to the pharmaceutical composition (e.g., as a drug combination or conjugated to the antibody or antigen binding fragment described herein).

Yet other aspects of the invention relate to the use of the isolated antibody or antigen binding fragment described herein in the detection of tumor cells or in the diagnosis of cancer. Tumors cells which may be particularly detected are those which expresses PSMA, especially if PSMA is located at the cell surface. The antibody or antigen binding fragment of the present invention are particularly useful for the detection of prostate tumor cells or of other PSMA-expressing cells such as neovasculature (in the case of psoriasis) including tumor neovasculature. Such tumor neovasculature is not only found in prostatic cancer but also in bladder and lung tumors and also in breast tumor, colon tumor and pancreatic tumor.

Other aspects of the invention relate to a composition which may comprise the antibody or antigen binding fragment described herein and a carrier.

Yet other aspects of the invention relate to the use of the isolated antibody or antigen binding fragment described herein in the treatment or diagnosis of cancer.

In addition to the active ingredients, a pharmaceutical composition may contain pharmaceutically acceptable carriers comprising water, PBS, salt solutions, gelatins, oils, alcohols, and other excipients and auxiliaries that facilitate processing of the active compounds into preparations that may be used pharmaceutically. In other instances, such preparations may be sterilized.

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's orfixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. “Pharmaceutically acceptable carriers” thus may include, without limitation, diluents (such as phosphate buffered saline buffers, water, saline), preservatives, solubilizers, emulsifiers, adjuvant and/or carriers, dispersion media, coatings, antibacterial and antifungal agents, isotonic and absorption delaying agents and the like. The use of such media and agents is well known in the art. Except insofar as any conventional media or agent is incompatible with antibodies of the present invention, its use in pharmaceutical compositions is contemplated.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above may be applied to any subject in need of such therapy, including, but not limited to, mammals such as dogs, cats, cows, horses, rabbits, monkeys, rats, mouse and humans.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

The present invention also relates to non-pharmaceutical composition which may contain the antibody or antigen binding fragment in aqueous solution or in other forms (e.g., freeze-dried, etc.). These non-pharmaceutical composition may have utility in in vitro assays or the like.

The term “treatment” for purposes of this disclosure refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The antibodies and antigen binding fragments may have therapeutic uses in the treatment of various diseases involving PSMA, such as prostate cancer or diseases involving neovasculature. In an exemplary embodiment, the antibodies or antigen binding fragments may interact with cancer cells that express PSMA and induce an immunological reaction by mediating humoral immunity, cellular immunity or complement-mediated immunity. In other instances, the antibodies and fragments may block the interaction of PSMA with its protein partners.

In certain instances, the antibodies and antigen binding fragments therein may be administered concurrently in combination with other treatments given for the same condition. As such, the antibodies may be administered with anti-mitotics (eg., taxanes), platinum-based agents (eg., cisplatin), DNA damaging agents (eg. Doxorubicin), and other anti-cancer therapies that are known to those skilled in the art. In other instances, the antibodies and antigen binding fragments therein may be administered with other therapeutic antibodies.

The present invention relates in a further aspect thereof to a method for inhibiting the growth of a PSMA-expressing cell, the method may comprise contacting the cell with an effective amount of the antibody or antigen binding fragment described herein. The use of a naked anti-PSMA antibody is especially contemplated herein.

The present invention also encompasses method of treating cancer or inhibiting the growth of a PSMA expressing cells in a mammal, the method may comprise administering the antibody or antigen binding fragment described herein to a mammal in need. The use of a naked anti-PSMA antibody is also especially contemplated herein.

It is to be understood herein that by “inhibiting” it is meant a process by which the growth of a PSMA-expressing cell may be reduced, delayed, prevented and/or impaired. The term “inhibiting” may also encompass cell death.

As it will become apparent from the method described herein and in accordance with the present invention, the method may be performed using a naked antibody or antigen binding fragment described herein. The method may also be performed using the naked antibody either alone or in combination with a second therapeutic molecule Furthermore, the method of the present invention may be carried out by using an antibody or antigen binding fragment which carries a diagnostic or therapeutic moiety.

In examplary embodiment of the invention the method may be carried out using antibodies which may comprise a portion capable of attracting immune effector cells (e.g. natural killer cells, macrophages, etc.). Such portion may be a Fc region derived from the same species or from another species, e.g. a mice antibody Fc region, a human antibody Fc region, etc.

The present invention relates in an additional aspect thereof to a method for treating cancer, which may comprise administering to a subject in need an effective amount of a pharmaceutical composition that may comprise the antibody or antigen binding fragment described herein.

According to the present invention, a “subject” may be a mammal. In accordance with the present invention, the mammal may be a human being. A subject in need thereof encompasses a subject that may need PSMA expressing-cell detection and/or a subject that may need cancer treatment (such as prostate cancer).

The term “cancer” is intended to mean any cellular malignancy whose unique trait is the loss of normal controls which may result in unregulated growth, lack of differentiation and/or ability to invade local tissues and metastasize. Cancer may develop in any tissue of any organ. In a non-limitative embodiment of the present invention, cancer is intended to include prostate cancer.

The present invention also encompasses method of detecting cancer or detecting a PSMA-expressing cells in a mammal, the method may comprise administering the antibody or antigen binding fragment described herein to a mammal in need.

According to the present invention, contacting and/or detecting may occur in vivo, ex vivo or in vitro. In vivo contacting involves administering to a subject an antibody (effective amount thereof) of the invention, for example in a composition and/or pharmaceutical composition. Ex vivo contact and/or in vitro contact involves contact with a biological sample obtained from a subject. A biological sample may comprise a sample of blood, serum and/or tissue biopsies.

It is to be understood herein that the PSMA expressing cell may be a normal cell or a cell which aberrantly expresses PSMA (e.g., a tumor cell).

According to the present invention, a cell which aberrantly expresses PSMA may be a cell that simply overexpresses PSMA without being tumoral. Alternatively and in accordance with the present invention, cell which aberrantly expresses PSMA may be a tumor cell.

In accordance with the present invention, a tumor cell may be a prostate cancer cell, an astrocytoma cell, a breast carcinoma cell, a carcinoid cell, a gastric carcinoma cell, a hepatocarcinoma cell, a Hodgkin's lymphoma cell, a leiomyoma cell, a lung adenocarcinoma cell, a lymphoma cell, a melanoma cell, an ovarian carcinoma cell, a rhabdosarcoma cell and/or a thyroid carcinoma cell. In an embodiment of the present invention, a tumor cell is a prostate cancer cell. In another embodiment, the prostate cancer cell may be a metastatic prostate cancer cell.

The present invention relates in another aspect thereof to a method for detecting a PSMA-expressing cell, the method may comprise contacting the cell with an antibody or antigen binding fragment described herein and detecting a complex formed by the antibody and the PSMA-expressing cell.

Another aspect of the invention relates a method for detecting PSMA, or a variant having at least 80% sequence identity with PSMA, the method may comprise contacting a cell or a sample (biopsy, serum, plasma, urine etc.) comprising or suspected of comprising PSMA or the PSMA variant with the antibody or antigen binding fragments described herein and measuring binding.

The sample may originate from a mammal (e.g., a human) which may have cancer (e.g., prostate cancer) or may be suspected of having cancer (e.g., prostate cancer). The sample may be a tissue sample obtained from the mammal or a cell culture supernatant.

In accordance with the invention the sample may be a biopsy, a serum sample, a plasma sample, a blood sample or ascitic fluid obtained from the mammal. The antibody or antigen binding fragment described herein may advantageously detect PSMA.

The method may comprise quantifying the complex formed by the antibody or antigen binding fragment bound to PSMA or to the PSMA variant.

The antibody or antigen binding fragment of the present invention may more particularly be used in the detection, diagnosis or treatment of prostate cancer.

Additional aspects of the invention relates to kits which may include one or more container containing one or more antibodies or antigen binding fragments described herein.

Kits of the present invention may additionally include, if desired, one or many conventional components, for example, containers that may comprise one or many excipients and/or pharmaceutically acceptable vehicles, or any other additional containers that may be evident to a person skilled in the art. A kit according to the present invention may also advantageously include instructions in the form of a pamphlet or of any other support, indicating the quantities to be used and/or administered and/or the instructions to mix given components.

Nucleic Acids, Vectors and Cells

Antibodies are usually made in cells allowing expression of the light chain and heavy chain expressed from a vector(s) comprising a nucleic acid sequence encoding the light chain and heavy chain.

The present invention therefore encompasses nucleic acids capable of encoding any of the CDRs, light chain variable domains, heavy chain variable domains, light chains, heavy chains described herein (including any of the variants).

Exemplary embodiments of nucleic acids of the present invention include nucleic acids encoding a light chain variable domain comprising:
a. a CDRL1 sequence selected from the group consisting of SEQ ID NO:1, SEQ ID NO: SEQ ID NO:14, SEQ ID NO:20, SEQ ID NO:26 and SEQ ID NO:32;
b. a CDRL2 sequence selected from the group consisting of SEQ ID NO:2, SEQ ID NO:9, SEQ ID NO: 15, SEQ ID NO:21, SEQ ID NO:27 and SEQ ID NO: 33, or;
c. a CDRL3 sequence selected from the group consisting of SEQ ID NO: 3, SEQ ID NO:10, SEQ ID NO:16, SEQ ID NO:22, SEQ ID NO:28 and SEQ ID NO: 34.

In accordance with the present invention, the nucleic acid may encode a light chain variable domain which may comprise at least two CDRs of a CDRL1, a CDRL2 or a CDRL3.

Also in accordance with the present invention, the nucleic acid may encode a light chain variable domain which may comprise one CDRL1, one CDRL2 and one CDRL3.

The present invention also relates to a nucleic acid encoding a heavy chain variable domain comprising:
a. a CDRH1 sequence selected from the group consisting of SEQ ID NO:4, SEQ ID NO:11, SEQ ID NO:17, SEQ ID NO:23, SEQ ID NO:29 and SEQ ID NO:35;
b. a CDRH2 sequence selected from the group consisting of SEQ ID NO:5, SEQ ID NO:6, SEQ ID NO:12, SEQ ID NO:18, SEQ ID NO:24, SEQ ID NO:30, SEQ ID NO:36 and SEQ ID NO:70 or;
c. a CDRH3 sequence selected from the group consisting of SEQ ID NO:7, SEQ ID NO:13, SEQ ID NO:19, SEQ ID NO:25, SEQ ID NO:31 and SEQ ID NO:37.

In accordance with the present invention, the nucleic acid may encode a heavy chain variable domain which may comprise at least two CDRs of a CDRH1, a CDRH2 or a CDRH3.

In accordance with the present invention, the nucleic acid may encode a heavy chain variable domain which may comprise one CDRH1, one CDRH2 and one CDRH3.

Also encompassed by the present invention are nucleic acids encoding the variant CDRs or the variant framework region, the vairant light chain, the variant heavy chain or the variant antibody or antigen binding fragments described herein.

In accordance with the present invention, the nucleic acid may encode a CDR comprising at least one amino acid substitution such as a conservative amino acid substitution.

In accordance with the present invention, the nucleic acid may encode a CDR comprising at least one conservative amino acid substitution in at least two of the CDRs.

In accordance with the present invention, the nucleic acid may encode a CDR comprising at least one conservative amino acid substitution in the 3 CDRs.

In accordance with the present invention, the nucleic acid may encode a CDR comprising at least two conservative amino acid substitution in at least one of the CDRs.

In accordance with the present invention, the nucleic acid may encode a CDR comprising at least two conservative amino acid substitution in at least two of the CDRs.

In accordance with the present invention, the nucleic acid may encode a CDR comprising at least two conservative amino acid substitution in the 3 CDRs.

Other aspects of the invention relate to a nucleic acid encoding a light chain variable domain having at least 80% sequence identity to a sequence selected from the group consisting of SEQ ID NO:38, SEQ ID NO:45, SEQ ID NO:47, SEQ ID NO:49, SEQ ID NO:51 and SEQ ID NO:53.

Yet other aspects of the invention relate to a nucleic acid encoding a heavy chain variable domain having at least 80% sequence identity to a sequence selected from the group consisting of SEQ ID NO:39, SEQ ID NO:40, SEQ ID NO:41, SEQ ID NO:42, SEQ ID NO:43, SEQ ID NO:44, SEQ ID NO:46, SEQ ID NO:48, SEQ ID NO:50, SEQ ID NO:52, SEQ ID NO:54 and sEQ UD NO:69.

In yet another aspect, the present invention relates to a vector comprising the nucleic acid described herein.

In accordance with the present invention, the vector may be an expression vector.

Vector that contains the elements for transcriptional and translational control of the inserted coding sequence in a particular host are known in the art. These elements may include regulatory sequences, such as enhancers, constitutive and inducible promoters, and 5′ and 3′ un-translated regions. Methods that are well known to those skilled in the art may be used to construct such expression vectors. These methods include in vitro recombinant DNA techniques, synthetic techniques, and in vivo genetic recombination.

In another aspect the present invention relates to an isolated cell which may comprise, the antibody or antigen binding fragment of the present invention, the nucleic acid or the vector described herein.

The isolated cell may comprise a nucleic acid encoding a light chain variable domain and a nucleic acid encoding a heavy chain variable domain either on separate vectors or on the same vector. The isolated cell may also comprise a nucleic acid encoding a light chain and a nucleic acid encoding a heavy chain either on separate vectors or on the same vector.

In accordance with the present invention, the cell may be capable of expressing, assembling and/or secreting an antibody or antigen binding fragment thereof.

In another aspect, the present invention provides a cell which may comprise and/or may express the antibody described herein.

In accordance with the invention, the cell may comprise a nucleic acid encoding a light chain variable domain and a nucleic acid encoding a heavy chain variable domain.

The cell may be capable of expressing, assembling and/or secreting an antibody or antigen binding fragment thereof.

### Other Embodiments

The present invention relates in further aspect thereof to an isolated polypeptide comprising an amino acid sequence at least 95% identical to any one of SEQ ID NO.: 1 to SEQ ID NO.:54.

In an exemplary embodiment, the isolated polypeptide may comprise a) an amino acid sequence at least 95% identical to SEQ ID NO.:14, b) an amino acid sequence at least 95% identical to SEQ ID NO.:15 and/or c) an amino acid sequence at least 95% identical to SEQ ID NO.:16.

In accordance with the present invention, the polypeptide may comprise sequentially (i.e., from the amino to the carboxy terminus) a) an amino acid sequence at least 95% identical to SEQ ID NO.:14, b) an amino acid sequence at least 95% identical to SEQ ID NO.:15 and c) an amino acid sequence at least 95% identical to SEQ ID NO.:16. Also in accordance with the present invention, the amino acid sequence of a) to b) may be separated by random amino acid sequence or by amino acid sequence similar or at least 95% identical with the amino acid sequence of SEQ ID NO.:47.

In another exemplary embodiment, the isolated polypeptide may comprise a) an amino acid sequence at least 95% identical to SEQ ID NO.:17, b) an amino acid sequence at least 95% identical to SEQ ID NO.:18 and/or c) an amino acid sequence at least 95% identical to SEQ ID NO.:19.

In accordance with the present invention, the polypeptide may comprise sequentially (i.e., from the amino to the carboxy terminus) a) an amino acid sequence at least 95% identical to SEQ ID NO.:17, b) an amino acid sequence at least 95% identical to SEQ ID NO.:18 and c) an amino acid sequence at least 95% identical to SEQ ID NO.:19. Also in accordance with the present invention, the amino acid sequence of a) to b) may be separated by random amino acid sequence or by amino acid sequence similar or at least 95% identical with the amino acid sequence of SEQ ID NO.:48.

The present invention relates in an aspect, to an antibody or an antigen binding fragment which may comprise:
a) A CDRH1 selected from the group consisting of a CDRH1 comprising SEQ ID NO:65 and a CDRH1 comprising SEQ ID NO:66;
b) A CDRH2 selected from the group consisting of a CDRH2 comprising SEQ ID NO:67 and a CDRH2 comprising SEQ ID NO:68;
c) A CDRH3 selected from the group consisting of a CDRH3 comprising SEQ ID NO:7, a CDRH3 comprising SEQ ID NO:13, a CDRH3 comprising SEQ ID NO:19, a CDRH3 comprising SEQ ID NO:25, a CDRH3 comprising SEQ ID NO:31 and a CDRH3 comprising SEQ ID NO:37, and;
d) A framework region selected from the group consisting of
   i. a framework region which may have, for example, at least 71, 72, 73 etc. (consecutive) amino acids of the framework region of SEQ ID NO:39;
   ii. a framework region which may have, for example, at least 67, 68, 69 etc. (consecutive) amino acids of the framework region of SEQ ID NO:46;
   iii. a framework region which may have, for example, at least 63, 64, 65, etc. (consecutive) amino acids of the framework region of SEQ ID NO:48;
   iv. a framework region which may have, for example, at least 70, 71, 72, etc. (consecutive) amino acids of the framework region of SEQ ID NO:50;
   v. a framework region which may have, for example, at least 72, 73, 74, etc. (consecutive) amino acids of the framework region of SEQ ID NO:52,
   vi. a framework region which may have, for example, at least 71, 72, 73, etc. (consecutive) amino acids of the framework region of SEQ ID NO:54 and;
   vii. a framework region which may have, for example, at least 68, 69, 70, etc. (consecutive) amino acids of the framework region of SEQ ID NO:69.

In accordance with the present invention, specific combination of CDRH1, CDRH2 and CDRH3 includes, for example, a CDRH1 comprising or consisting of SEQ ID NO:65 or SEQ ID NO:66, a CDRH2 comprising or consisting of SEQ ID NO:67 and a CDRH3 comprising or consisting of SEQ ID NO:7. In accordance with the present invention, another specific combination of CDRH1, CDRH2 and CDRH3 includes, for example, a CDRH1 comprising or consisting of SEQ ID NO:65, a CDRH2 comprising or consisting of SEQ ID NO:36 and a CDRH3 comprising or consisting of SEQ ID NO:37. In accordance with the present invention, yet another specific combination of CDRH1, CDRH2 and CDRH3 includes, for example, a CDRH1 comprising or consisting of SEQ ID NO:65 or SEQ ID NO:66, a CDRH2 comprising or consisting of SEQ ID NO:67 and a CDRH3 comprising or consisting of SEQ ID NO:13, SEQ ID NO:19 or SEQ ID NO:25.

In accordance with the present invention, the antibody or antigen binding fragment may further comprise a complementary light chain variable domain.

Also in accordance with the present invention, the antibody or antigen binding fragment may further comprise:
a) A CDRL1 comprising SEQ ID NO:61;
b) A CDRL2 selected from the group consisting of a CDRL2 comprising SEQ ID NO:62 and a CDRL2 comprising SEQ ID NO:63;
c) A CDRL3 comprising SEQ ID NO:64; and
d) A framework region selected from the group consisting of
   i. a framework region which may have, for example, at least 67, 68, 69, etc. (consecutive) amino acids of the framework region of SEQ ID NO:38;
   ii. a framework region which may have, for example, at least 67, 68, 69, etc. (consecutive) amino acids of the framework region of SEQ ID NO:45;
   iii. a framework region which may have, for example, at least 70, 71, 72, etc. (consecutive) amino acids of the framework region of SEQ ID NO:47;
   iv. a framework region which may have, for example, at least 66, 67, 68, etc. (consecutive) amino acids of the framework region of SEQ ID NO:49;
   v. a framework region which may have, for example, at least 66, 67, 68, etc. (consecutive) amino acids of the framework region of SEQ ID NO:51, and;
   vi. a framework region which may have, for example, at least 66, 67, 68, etc. (consecutive) amino acids of the framework region of SEQ ID NO:53.

In another aspect, the present invention relates to an antibody or an antigen binding fragment which may comprise:
a) A CDRL1 comprising SEQ ID NO:61;
b) A CDRL2 selected from the group consisting of a CDRL2 comprising SEQ ID NO:62 and a CDRL2 comprising SEQ ID NO:63;
c) A CDRL3 comprising SEQ ID NO:64; and
d) A framework region selected from the group consisting of
   i. a framework region which may have, for example, at least 67, 68, 69, etc. (consecutive) amino acids of the framework region of SEQ ID NO:38;
   ii. a framework region which may have, for example, at least 67, 68, 69, etc. (consecutive) amino acids of the framework region of SEQ ID NO:45;
   iii. a framework region which may have, for example, at least 70, 71, 72, etc. (consecutive) amino acids of the framework region of SEQ ID NO:47;
   iv. a framework region which may have, for example, at least 66, 67, 68, etc. (consecutive) amino acids of the framework region of SEQ ID NO:49;
   v. a framework region which may have, for example, at least 66, 67, 68, etc. (consecutive) amino acids of the framework region of SEQ ID NO:51, and;
   vi. a framework region which may have, for example, at least 66, 67, 68, etc. (consecutive) amino acids of the framework region of SEQ ID NO:53.

In accordance with the present invention, specific combination of CDRL1, CDRL2 and CDRL3 includes, for example, a CDRL1 comprising or consisting of SEQ ID NO:61, a CDRL2 comprising or consisting of SEQ ID NO:62 and a CDRL3 comprising or consisting of SEQ ID NO:64. In accordance with the present invention, another specific combination of CDRL1, CDRL2 and CDRL3 includes, for example, a CDRL1 comprising or consisting of SEQ ID NO:61, a CDRL2 comprising or consisting of SEQ ID NO:63 and a CDRL3 comprising or consisting of SEQ ID NO:64.

In accordance with the present invention, the antibody or antigen binding fragment may further comprise a complementary heavy chain variable domain.

In accordance with a specific embodiment, the antibody or antigen binding fragment may comprise for example, a CDRH3 comprising SEQ ID NO:19.

In such instance, the antibody or antigen binding fragment may comprise a CDRH1 comprising SEQ ID NO:17, a CDRH2 comprising SEQ ID NO:18 and a CDRH3 comprising SEQ ID NO:19. In accordance with an embodiment of the invention, the antibody or antigen binding fragment may comprise a framework region which may have, for example, at least 63, 64, 65, etc. (consecutive) amino acids of the framework region of SEQ ID NO:48. The antibody or antigen binding fragment may comprise a complementary light chain variable region. The antibody or antigen binding fragment of the present invention may comprise, for example, a CDRL1 comprising SEQ ID NO:14, a CDRL2 comprising SEQ ID NO:15 and a CDRL3 comprising SEQ ID NO:16. Also in accordance with the present invention, the antibody or antigen binding fragment may comprise a framework region which may have, for example, at least 70. 71, 72, etc. (consecutive) amino acids of the framework region of SEQ ID NO:47.

In accordance with another specific embodiment, the antibody or antigen binding fragment may comprise a CDRH3 comprising SEQ ID NO:7.

In such instance, the antibody or antigen binding fragment may comprise a CDRH1 comprising SEQ ID NO:4, a CDRH2 comprising SEQ ID NO:5 and a CDRH3 comprising SEQ ID NO:7. In accordance with an embodiment of the invention, the antibody or antigen binding fragment may comprise a framework region which may have, for example, at least 66, 67, 68, etc. (consecutive) amino acids of the framework region of SEQ ID NO:40. The antibody or antigen binding fragment may comprise a complementary light chain variable region. The antibody or antigen binding fragment of the present invention may comprise, for example, a CDRL1 comprising SEQ ID NO:8, a CDRL2 comprising SEQ ID NO:9 and a CDRL3 comprising SEQ ID NO:10. Also in accordance with the present invention, the antibody or antigen binding fragment may comprise a framework region which may have, for example, at least 67, 68, 69, etc. (consecutive) amino acids of the framework region of SEQ ID NO:38.

Alternatively, in such instance, the antibody or antigen binding fragment may comprise a CDRH1 comprising SEQ ID NO:4, a CDRH2 comprising SEQ ID NO:6 and a CDRH3 comprising SEQ ID NO:7. In accordance with an embodiment of the invention, the antibody or antigen binding fragment may comprise a framework region which may have, for example, at least 71, 72, 73, etc. (consecutive) amino acids of the framework region of SEQ ID NO:39. The antibody or antigen binding fragment may comprise a complementary light chain variable region. The antibody or antigen binding fragment of the present invention may comprise, for example, a CDRL1 comprising SEQ ID NO:8, a CDRL2 comprising SEQ ID NO:9 and a CDRL3 comprising SEQ ID NO:10. Also in accordance with the present invention, the antibody or antigen binding fragment may comprise a framework region which may have, for example, at least 67, 68, 69, etc. (consecutive) amino acids of the framework region of SEQ ID NO:38.

In accordance with yet another specific embodiment, the antibody or antigen binding fragment may comprise for example, a CDRH3 comprising SEQ ID NO:13.

In such instance, the antibody or antigen binding fragment may comprise a CDRH1 comprising SEQ ID NO:11, a CDRH2 comprising SEQ ID NO:12, a CDRH3 comprising SEQ ID NO:13. In accordance with an embodiment of the invention, the antibody or antigen binding fragment may comprise framework region which may have, for example, at least 67, 68, 69, etc. (consecutive) amino acids of the framework region of SEQ ID NO:46. The antibody or antigen binding fragment may comprise a complementary light chain variable region. The antibody or antigen binding fragment of the present invention may comprise, for example, a CDRL1 comprising SEQ ID NO:8, a CDRL2 comprising SEQ ID NO:9 and a CDRL3 comprising SEQ ID NO:10. Also in accordance with the present invention, the antibody or antigen binding fragment may comprise a framework region which may have, for example, at least 67, 68, 69, etc. (consecutive) amino acids of the framework region of SEQ ID NO:45.

In accordance with an additional specific embodiment, the antibody or antigen binding fragment may comprise for example, CDRH3 comprising SEQ ID NO:25.

In such instance, the antibody or antigen binding fragment may comprise a CDRH1 comprising SEQ ID NO:23, a CDRH2 comprising SEQ ID NO:24, a CDRH3 comprising SEQ ID NO:25. In accordance with an embodiment of the invention, the antibody or antigen binding fragment may comprise a framework region which may have, for example, at least 70, 71, 72, etc. (consecutive) amino acids of the framework region of SEQ ID NO:50. The antibody or antigen binding fragment may comprise a complementary light chain variable region. The antibody or antigen binding fragment of the present invention may comprise, for example, a CDRL1 comprising SEQ ID NO:20, a CDRL2 comprising SEQ ID NO:21 and a CDRL3 comprising SEQ ID NO:22. Also in accordance with the present invention, the antibody or antigen binding fragment may comprise a framework region which may have, for example, at least 66, 67, 68, etc. (consecutive) amino acids of the framework region of SEQ ID NO:49.

Alternatively, in such instance, the antibody or antigen binding fragment may comprise a CDRH1 comprising SEQ ID NO:23, a CDRH2 comprising SEQ ID NO:70, a CDRH3 comprising SEQ ID NO:25. In accordance with an embodiment of the invention, the antibody or antigen binding fragment may comprise a framework region which may have, for example, at least 68, 69, 70, etc. (consecutive) amino acids of the framework region of SEQ ID NO:69. The antibody or antigen binding fragment may comprise a complementary light chain variable region. The antibody or antigen binding fragment of the present invention may comprise, for example, a CDRL1 comprising SEQ ID NO:20, a CDRL2 comprising SEQ ID NO:21 and a CDRL3 comprising SEQ ID NO:22. Also in accordance with the present invention, the antibody or antigen binding fragment may comprise a framework region which may have, for example, at least 66, 67, 68, etc. (consecutive) amino acids of the framework region of SEQ ID NO:49.

In accordance with a further specific embodiment, the antibody or antigen binding fragment may comprise for example, a CDRH3 comprising SEQ ID NO:31.

In such instance, the antibody or antigen binding fragment may comprise a CDRH1 comprising SEQ ID NO:29, a CDRH2 comprising SEQ ID NO:30, a CDRH3 comprising SEQ ID NO:31. In accordance with an embodiment of the invention, the antibody or antigen binding fragment may comprise a framework region which may have, for example, at least 72, 73, 74, etc. (consecutive) amino acids of the framework region of SEQ ID NO:52. The antibody or antigen binding fragment may comprise a complementary light chain variable region. The antibody or antigen binding fragment of the present invention may comprise, for example, a CDRL1 comprising SEQ ID NO:26, a CDRL2 comprising SEQ ID NO:27 and a CDRL3 comprising SEQ ID NO:28. Also in accordance with the present invention, the antibody or antigen binding fragment may comprise a framework region which may have, for example, at least 68, 67, 68, etc. (consecutive) amino acids of the framework region of SEQ ID NO:51.

In accordance with yet a further a specific embodiment, the antibody or antigen binding fragment may comprise for example, a CDRH3 comprising SEQ ID NO:37.

In such instance, the antibody or antigen binding fragment may comprise a CDRH1 comprising SEQ ID NO:35, a CDRH2 comprising SEQ ID NO:36, a CDRH3 comprising SEQ ID NO:37. In accordance with an embodiment of the invention, the antibody or antigen binding fragment may comprise a framework region which may have, for example, at least 71, 72, 73, etc. (consecutive) amino acids of the framework region of SEQ ID NO:54. The antibody or antigen binding fragment may comprise a complementary light chain variable region. The antibody or antigen binding fragment of the present invention may comprise, for example, a CDRL1 comprising SEQ ID NO:32, a CDRL2 comprising SEQ ID NO:33 and a CDRL3 comprising SEQ ID NO:34. Also in accordance with the present invention, the antibody or antigen binding fragment may comprise a framework region which may have, for example, at least 66, 67, 68, etc. (consecutive) amino acids of the framework region of SEQ ID NO:53.

Other aspects of the invention relate to the use of the (e.g., naked or not) antibody or antigen binding fragment described herein, for reducing the growth or a tumor cell. The tumor cell may be for example, a prostate tumor cell.

Yet other aspects of the invention relate to the use of the antibody or antigen binding fragment described herein, for detecting a PSMA-expressing cell.

In accordance with the present invention, the PSMA-expressing cell may be a tumor cell. Alternatively, the PSMA-expressing cell may be a cell of a neovasculature (non-tumor, e.g., psoriasis) including cell tumor neovasculature.

Additional aspects of the invention, relate to a pharmaceutical composition which may comprise the antibody or antigen binding fragment described herein and a pharmaceutically acceptable carrier.

In accordance with the present invention, the pharmaceutical composition may further comprise an anticancer drug.

Yet additional aspects of the invention relate to a conjugate which may comprise the antibody or antigen binding fragment described herein and a detectable moiety.

In accordance with the present invention, the conjugate may comprise the antibody or antigen binding fragment described herein and a therapeutic moiety.

Further aspects of the invention relate to an antibody capable of binding to PSMA which may be capable of lowering the growth of a cell expressing PSMA without being conjugated or associated with a drug.

The antibody or antigen binding fragment of the invention, may bind, for example, to an extracellular portion of PSMA.

Yet further aspects of the invention, relate to the use of a naked antibody capable of binding to PSMA in the preparation of a medicament for reducing the growth of prostate tumor cells.

In accordance with the present invention, the naked antibody may comprise a heavy chain variable domain comprising:
a) i. a CDRH1 which may comprise SEQ ID NO:4 or a variant having one or two amino acid substitutions, deletions or insertions in SEQ ID NO:4; ii. a CDRH2 which may comprise SEQ ID NO:5 or SEQ ID NO:6 or a variant having one or two amino acid substitutions, deletions or insertions in SEQ ID NO:5 or SEQ ID NO:6; iii. a CDRH3 which may comprise SEQ ID NO:7 or a variant having one or two amino acid substitutions, deletions or insertions in SEQ ID NO:7, and; iv. a framework region which may be at least 75% identical to the framework region of SEQ ID NO:39;
b) i. a CDRH1 which may comprise SEQ ID NO:11 or a variant having one or two amino acid substitutions, deletions or insertions in SEQ ID NO:11; ii. a CDRH2 which may comprise SEQ ID NO:12 or a variant having one or two amino acid substitutions, deletions or insertions in SEQ ID NO:12; iii. a CDRH3 which may comprise SEQ ID NO:13 or a variant having one or two amino acid substitutions, deletions or insertions in SEQ ID NO:13, and; iv. a framework region which may be at least 75% identical to the framework region of SEQ ID NO:46;
c) i. a CDRH1 which may comprise SEQ ID NO:17 or a variant having one or two amino acid substitutions, deletions or insertions in SEQ ID NO:17; ii. a CDRH2 which may comprise SEQ ID NO:18 or a variant having one or two amino acid substitutions, deletions or insertions in SEQ ID NO:18; iii. a CDRH3 which may comprise SEQ ID NO:19 or a variant having one or two amino acid substitutions, deletions or insertions in SEQ ID NO:19, and; iv. a framework region which may be at least 75% identical to the framework region of SEQ ID NO:48;
d) i. a CDRH1 which may comprise SEQ ID NO:23 or a variant having one or two amino acid substitutions, deletions or insertions in SEQ ID NO:23; ii. a CDRH2 which may comprise SEQ ID NO:24 or a variant having one or two amino acid substitutions, deletions or insertions in SEQ ID NO:24; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:50;
e) i. a CDRH1 which may comprise SEQ ID NO:23 or a variant having one or two amino acid substitutions, deletions or insertions in SEQ ID NO:23; ii. a CDRH2 which may comprise SEQ ID NO:70 or a variant having one or two amino acid substitutions, deletions or insertions in SEQ ID NO:70; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:69;
f) i. a CDRH1 which may comprise SEQ ID NO:29 or a variant having one or two amino acid substitutions, deletions or insertions in SEQ ID NO:29; ii. a CDRH2 which may comprise SEQ ID NO:30 or a variant having one or two amino acid substitutions, deletions or insertions in SEQ ID NO:30; iii. a CDRH3 which may comprise SEQ ID NO:31 or a variant having one or two amino acid substitutions, deletions or insertions in SEQ ID NO:31, and; iv. a framework region at least 75% identical to the framework region of SEQ ID NO:52, or;
g) i. a CDRH1 which may comprise SEQ ID NO:35 or a variant having one or two amino acid substitutions, deletions or insertions in SEQ ID NO:35; ii. a CDRH2 which may comprise SEQ ID NO:36 or a variant having one or two amino acid substitutions, deletions or insertions in SEQ ID NO:36; iii. a CDRH3 which may comprise SEQ ID NO:37 or a variant having one or two amino acid substitutions, deletions or insertions in SEQ ID NO:37, and; iv. a framework region which may be at least 75% identical to the framework region of SEQ ID NO:54.

Also in accordance with the present invention, the naked antibody may comprise a complementary light chain variable region.

Such complementary light chain variable region may be selected, for example, from the group consisting of:
a) i. a CDRL1 which may comprise SEQ ID NO:1 or a variant having one or two amino acid substitutions, deletions or insertions in SEQ ID NO:1; ii. a CDRL2 which may comprise SEQ ID NO:2 or a variant having one or two amino acid substitutions, deletions or insertions in SEQ ID NO:2; iii. a CDRL3 which may comprise SEQ ID NO:3 or a variant having one or two amino acid substitutions, deletions or insertions in SEQ ID NO:3, and; iv. a framework region which may be at least 75% identical to the framework region of SEQ ID NO:38;
b) i. a CDRL1 which may comprise SEQ ID NO:8 or a variant having one or two amino acid substitutions, deletions or insertions in SEQ ID NO:8; ii. a CDRL2 which may comprise SEQ ID NO:9 or a variant having one or two amino acid substitutions, deletions or insertions in SEQ ID NO:9; iii. a CDRL3 which may comprise SEQ ID NO:10 or a variant having one or two amino acid substitutions, deletions or insertions in SEQ ID NO:10, and; iv. a framework region which may be at least 75% identical to the framework region of SEQ ID NO:45;
c) i. a CDRL1 which may comprise SEQ ID NO:14 or a variant having one or two amino acid substitutions, deletions or insertions in SEQ ID NO:14; ii. a CDRL2 which may comprise SEQ ID NO:15 or a variant having one or two amino acid substitutions, deletions or insertions in SEQ ID NO:15; iii. a CDRL3 which may comprise SEQ ID NO:16 or a variant having one or two amino acid substitutions, deletions or insertions in SEQ ID NO:16, and; iv. a framework region which may be at least 75% identical to the framework region of SEQ ID NO:47;
d) i. a CDRL1 which may comprise SEQ ID NO:20 or a variant having one or two amino acid substitutions, deletions or insertions in SEQ ID NO:20; ii. a CDRL2 which may comprise SEQ ID NO:21 or a variant having one or two amino acid substitutions, deletions or insertions in SEQ ID NO:21; iii. a CDRL3 which may comprise SEQ ID NO:22 or a variant having one or two amino acid substitutions, deletions or insertions in SEQ ID NO:22, and; iv. a framework region which may be at least 75% identical to the framework region of SEQ ID NO:49;
e) i. a CDRL1 which may comprise SEQ ID NO:26 or a variant having one or two amino acid substitutions, deletions or insertions in SEQ ID NO:26; ii. a CDRL2 which may comprise SEQ ID NO:27 or a variant having one or two amino acid substitutions, deletions or insertions in SEQ ID NO:27; iii. a CDRL3 which may comprise SEQ ID NO:28 or a variant having one or two amino acid substitutions, deletions or insertions in SEQ ID NO:28, and; iv. a framework region which may be at least 75% identical to the framework region of SEQ ID NO:51, or;
f) i. a CDRL1 which may comprise SEQ ID NO:32 or a variant having one or two amino acid substitutions, deletions or insertions in SEQ ID NO:32; ii. a CDRL2 which may comprise SEQ ID NO:33 or a variant having one or two amino acid substitutions, deletions or insertions in SEQ ID NO:33; iii. a CDRL3 which may comprise SEQ ID NO:34 or a variant having one or two amino acid substitutions, deletions or insertions in SEQ ID NO:34, and; iv. a framework region which may be at least 75% identical to the framework region of SEQ ID NO:53.

In accordance with the present invention, the naked antibody may be used, for example, in a combination with a cytotoxic drug.

Also in accordance with the present invention, if desired, the naked antibody may optionally be conjugated with a cytotoxic drug.

In an additional aspect, the present invention relate to the use of a naked antibody capable of binding to PSMA for reducing the growth of prostate cancer cells.

In accordance with the present invention, the naked antibody may comprise a heavy chain variable domain comprising:
a) i. a CDRH1 which may comprise SEQ ID NO:4 or a variant having one or two amino acid substitutions, deletions or insertions in SEQ ID NO:4; ii. a CDRH2 which may comprise SEQ ID NO:5 or SEQ ID NO:6 or a variant having one or two amino acid substitutions, deletions or insertions in SEQ ID NO:5 or SEQ ID NO:6; iii. a CDRH3 which may comprise SEQ ID NO:7 or a variant having one or two amino acid substitutions, deletions or insertions in SEQ ID NO:7, and; iv. a framework region which may be at least 75% identical to the framework region of SEQ ID NO:39;
b) i. a CDRH1 which may comprise SEQ ID NO:11 or a variant having one or two amino acid substitutions, deletions or insertions in SEQ ID NO:11; ii. a CDRH2 which may comprise SEQ ID NO:12 or a variant having one or two amino acid substitutions, deletions or insertions in SEQ ID NO:12; iii. a CDRH3 which may comprise SEQ ID NO:13 or a variant having one or two amino acid substitutions, deletions or insertions in SEQ ID NO:13, and; iv. a framework region which may be at least 75% identical to the framework region of SEQ ID NO:46;
c) i. a CDRH1 which may comprise SEQ ID NO:17 or a variant having one or two amino acid substitutions, deletions or insertions in SEQ ID NO:17; ii. a CDRH2 which may comprise SEQ ID NO:18 or a variant having one or two amino acid substitutions, deletions or insertions in SEQ ID NO:18; iii. a CDRH3 which may comprise SEQ ID NO:19 or a variant having one or two amino acid substitutions, deletions or insertions in SEQ ID NO:19, and; iv. a framework region which may be at least 75% identical to the framework region of SEQ ID NO:48;
d) i. a CDRH1 which may comprise SEQ ID NO:23 or a variant having one or two amino acid substitutions, deletions or insertions in SEQ ID NO:23; ii. a CDRH2 which may comprise SEQ ID NO:24 or a variant having one or two amino acid substitutions, deletions or insertions in SEQ ID NO:24; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:50;
e) i. a CDRH1 which may comprise SEQ ID NO:23 or a variant having one or two amino acid substitutions, deletions or insertions in SEQ ID NO:23; ii. a CDRH2 which may comprise SEQ ID NO:70 or a variant having one or two amino acid substitutions, deletions or insertions in SEQ ID NO:70; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:69;
f) i. a CDRH1 which may comprise SEQ ID NO:29 or a variant having one or two amino acid substitutions, deletions or insertions in SEQ ID NO:29; ii. a CDRH2 which may comprise SEQ ID NO:30 or a variant having one or two amino acid substitutions, deletions or insertions in SEQ ID NO:30; iii. a CDRH3 which may comprise SEQ ID NO:31 or a variant having one or two amino acid substitutions, deletions or insertions in SEQ ID NO:31, and; iv. a framework region which may be at least 75% identical to the framework region of SEQ ID NO:52, or;
g) i. a CDRH1 which may comprise SEQ ID NO:35 or a variant having one or two amino acid substitutions, deletions or insertions in SEQ ID NO:35; ii. a CDRH2 which may comprise SEQ ID NO:36 or a variant having one or two amino acid substitutions, deletions or insertions in SEQ ID NO:36; iii. a CDRH3 which may comprise SEQ ID NO:37 or a variant having one or two amino acid substitutions, deletions or insertions in SEQ ID NO:37, and; iv. a framework region which may be at least 75% identical to the framework region of SEQ ID NO:54.

Also in accordance with the present invention, the naked antibody may comprise a complementary light chain variable region.

Such complementary light chain variable region may be selected, for example, from the group consisting of:
a) i. a CDRL1 which may comprise SEQ ID NO:1 or a variant having one or two amino acid substitutions, deletions or insertions in SEQ ID NO:1; ii. a CDRL2 which may comprise SEQ ID NO:2 or a variant having one or two amino acid substitutions, deletions or insertions in SEQ ID NO:2; iii. a CDRL3 which may comprise SEQ ID NO:3 or a variant having one or two amino acid substitutions, deletions or insertions in SEQ ID NO:3, and; iv. a framework region which may be at least 75% identical to the framework region of SEQ ID NO:38;
b) i. a CDRL1 which may comprise SEQ ID NO:8 or a variant having one or two amino acid substitutions, deletions or insertions in SEQ ID NO:8; ii. a CDRL2 which may comprise SEQ ID NO:9 or a variant having one or two amino acid substitutions, deletions or insertions in SEQ ID NO:9; iii. a CDRL3 which may comprise SEQ ID NO:10 or a variant having one or two amino acid substitutions, deletions or insertions in SEQ ID NO:10, and; iv. a framework region which may be at least 75% identical to the framework region of SEQ ID NO: 45;
c) i. a CDRL1 which may comprise SEQ ID NO:14 or a variant having one or two amino acid substitutions, deletions or insertions in SEQ ID NO:14; ii. a CDRL2 which may comprise SEQ ID NO:15 or a variant having one or two amino acid substitutions, deletions or insertions in SEQ ID NO:15; iii. a CDRL3 which may comprise SEQ ID NO:16 or a variant having one or two amino acid substitutions, deletions or insertions in SEQ ID NO:16, and; iv. a framework region which may be at least 75% identical to the framework region of SEQ ID NO: 47;
d) i. a CDRL1 which may comprise SEQ ID NO:20 or a variant having one or two amino acid substitutions, deletions or insertions in SEQ ID NO:20; ii. a CDRL2 which may comprise SEQ ID NO:21 or a variant having one or two amino acid substitutions, deletions or insertions in SEQ ID NO:21; iii. a CDRL3 which may comprise SEQ ID NO:22 or a variant having one or two amino acid substitutions, deletions or insertions in SEQ ID NO:22, and; iv. a framework region which may be at least 75% identical to the framework region of SEQ ID NO:49;
e) i. a CDRL1 which may comprise SEQ ID NO:26 or a variant having one or two amino acid substitutions, deletions or insertions in SEQ ID NO:26; ii. a CDRL2 which may comprise SEQ ID NO:27 or a variant having one or two amino acid substitutions, deletions or insertions in SEQ ID NO:27; iii. a CDRL3 which may comprise SEQ ID NO:28 or a variant having one or two amino acid substitutions, deletions or insertions in SEQ ID NO:28, and; iv. a framework region which may be at least 75% identical to the framework region of SEQ ID NO:51, or;
f) i. a CDRL1 which may comprise SEQ ID NO:32 or a variant having one or two amino acid substitutions, deletions or insertions in SEQ ID NO:32; ii. a CDRL2 which may comprise SEQ ID NO:33 or a variant having one or two amino acid substitutions, deletions or insertions in SEQ ID NO:33; iii. a CDRL3 which may comprise SEQ ID NO:34 or a variant having one or two amino acid substitutions, deletions or insertions in SEQ ID NO:34, and; iv. a framework region which may be at least 75% identical to the framework region of SEQ ID NO:53.

In accordance with the present invention, the naked antibody may be used in combination with a cytotoxic drug.

Also in accordance with the present invention, the naked antibody may optionally be conjugated with a cytotoxic drug, if desired.

In a further aspect, the present invention relate to a method for reducing the growth of prostate cancer cells, the method may comprise, for example, administering a naked antibody capable of binding to PSMA to a mammal in need.

The naked antibody may comprise, for example, a heavy chain variable domain comprising:
a) i. a CDRH1 which may comprise SEQ ID NO:4 or a variant having one or two amino acid substitutions, deletions or insertions in SEQ ID NO:4; ii. a CDRH2 which may comprise SEQ ID NO:5 or SEQ ID NO:6 or a variant having one or two amino acid substitutions, deletions or insertions in SEQ ID NO:5 or SEQ ID NO:6; iii. a CDRH3 which may comprise SEQ ID NO:7 or a variant having one or two amino acid substitutions, deletions or insertions in SEQ ID NO:7, and; iv. a framework region which may be at least 75% identical to the framework region of SEQ ID NO: 39;
b) i. a CDRH1 which may comprise SEQ ID NO:11 or a variant having one or two amino acid substitutions, deletions or insertions in SEQ ID NO:11; ii. a CDRH2 which may comprise SEQ ID NO:12 or a variant having one or two amino acid substitutions, deletions or insertions in SEQ ID NO:12; iii. a CDRH3 which may comprise SEQ ID NO:13 or a variant having one or two amino acid substitutions, deletions or insertions in SEQ ID NO:13, and; iv. a framework region which may be at least 75% identical to the framework region of SEQ ID NO:46;
c) i. a CDRH1 which may comprise SEQ ID NO:17 or a variant having one or two amino acid substitutions, deletions or insertions in SEQ ID NO:17; ii. a CDRH2 which may comprise SEQ ID NO:18 or a variant having one or two amino acid substitutions, deletions or insertions in SEQ ID NO:18; iii. a CDRH3 which may comprise SEQ ID NO:19 or a variant having one or two amino acid substitutions, deletions or insertions in SEQ ID NO:19, and; iv. a framework region which may be at least 75% identical to the framework region of SEQ ID NO:48;
d) i. a CDRH1 which may comprise SEQ ID NO:23 or a variant having one or two amino acid substitutions, deletions or insertions in SEQ ID NO:23; ii. a CDRH2 which may comprise SEQ ID NO:24 or a variant having one or two amino acid substitutions, deletions or insertions in SEQ ID NO:24; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:50;
e) i. a CDRH1 which may comprise SEQ ID NO:23 or a variant having one or two amino acid substitutions, deletions or insertions in SEQ ID NO:23; ii. a CDRH2 which may comprise SEQ ID NO:70 or a variant having one or two amino acid substitutions, deletions or insertions in SEQ ID NO:70; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:69;
f) i. a CDRH1 which may comprise SEQ ID NO:29 or a variant having one or two amino acid substitutions, deletions or insertions in SEQ ID NO:29; ii. a CDRH2 which may comprise SEQ ID NO:30 or a variant having one or two amino acid substitutions, deletions or insertions in SEQ ID NO:30; iii. a CDRH3 which may comprise SEQ ID NO:31 or a variant having one or two amino acid substitutions, deletions or insertions in SEQ ID NO:31, and; iv. a framework region which may be at least 75% identical to the framework region of SEQ ID NO:52, or;
g) i. a CDRH1 which may comprise SEQ ID NO:35 or a variant having one or two amino acid substitutions, deletions or insertions in SEQ ID NO:35; ii. a CDRH2 which may comprise SEQ ID NO:36 or a variant having one or two amino acid substitutions, deletions or insertions in SEQ ID NO:36; iii. a CDRH3 which may comprise SEQ ID NO:37 or a variant having one or two amino acid substitutions, deletions or insertions in SEQ ID NO:37, and; iv. a framework region which may be at least 75% identical to the framework region of SEQ ID NO:54.

In accordance with the present invention the naked antibody may comprise a complementary light chain variable region.

Such complementary light chain variable region may be selected, for example, from the group consisting of:
a) i. a CDRL1 which may comprise SEQ ID NO:1 or a variant having one or two amino acid substitutions, deletions or insertions in SEQ ID NO:1; ii. a CDRL2 which may comprise SEQ ID NO:2 or a variant having one or two amino acid substitutions, deletions or insertions in SEQ ID NO:2; iii. a CDRL3 which may comprise SEQ ID NO:3 or a variant having one or two amino acid substitutions, deletions or insertions in SEQ ID NO:3, and; iv. a framework region which may be at least 75% identical to the framework region of SEQ ID NO:38;
b) i. a CDRL1 which may comprise SEQ ID NO:8 or a variant having one or two amino acid substitutions, deletions or insertions in SEQ ID NO:8; ii. a CDRL2 which may comprise SEQ ID NO:9 or a variant having one or two amino acid substitutions, deletions or insertions in SEQ ID NO:9; iii. a CDRL3 which may comprise SEQ ID NO:10 or a variant having one or two amino acid substitutions, deletions or insertions in SEQ ID NO:10, and; iv. a framework region which may be at least 75% identical to the framework region of SEQ ID NO:45;
c) i. a CDRL1 which may comprise SEQ ID NO:14 or a variant having one or two amino acid substitutions, deletions or insertions in SEQ ID NO:14; ii. a CDRL2 which may comprise SEQ ID NO:15 or a variant having one or two amino acid substitutions, deletions or insertions in SEQ ID NO:15; iii. a CDRL3 which may comprise SEQ ID NO:16 or a variant having one or two amino acid substitutions, deletions or insertions in SEQ ID NO:16, and; iv. a framework region which may be at least 75% identical to the framework region of SEQ ID NO:47;
d) i. a CDRL1 which may comprise SEQ ID NO:20 or a variant having one or two amino acid substitutions, deletions or insertions in SEQ ID NO:20; ii. a CDRL2 which may comprise SEQ ID NO:21 or a variant having one or two amino acid substitutions, deletions or insertions in SEQ ID NO:21; iii. a CDRL3 which may comprise SEQ ID NO:22 or a variant having one or two amino acid substitutions, deletions or insertions in SEQ ID NO:22, and; iv. a framework region which may be at least 75% identical to the framework region of SEQ ID NO:49;
e) i. a CDRL1 which may comprise SEQ ID NO:26 or a variant having one or two amino acid substitutions, deletions or insertions in SEQ ID NO:26; ii. a CDRL2 which may comprise SEQ ID NO:27 or a variant having one or two amino acid substitutions, deletions or insertions in SEQ ID NO:27; iii. a CDRL3 which may comprise SEQ ID NO:28 or a variant having one or two amino acid substitutions, deletions or insertions in SEQ ID NO:28, and; iv. a framework region which may be at least 75% identical to the framework region of SEQ ID NO:51, or;
f) i. a CDRL1 which may comprise SEQ ID NO:32 or a variant having one or two amino acid substitutions, deletions or insertions in SEQ ID NO:32; ii. a CDRL2 which may comprise SEQ ID NO:33 or a variant having one or two amino acid substitutions, deletions or insertions in SEQ ID NO:33; iii. a CDRL3 which may comprise SEQ ID NO:34 or a variant having one or two amino acid substitutions, deletions or insertions in SEQ ID NO:34, and; iv. a framework region which may be at least 75% identical to the framework region of SEQ ID NO:53.

In accordance with the present invention, the naked antibody may be used in combination with a cytotoxic drug.

Also accordance with the present invention, the naked antibody may optionally be conjugated with a cytotoxic drug.

In yet a further aspect, the present invention relate to a pharmaceutical composition for reducing the growth of prostate cancer cells. The pharmaceutical composition may comprise a naked antibody capable of binding to PSMA and a pharmaceutically acceptable carrier.

In accordance with the present invention, the naked antibody may comprise, for example, a heavy chain variable domain comprising:
a) i. a CDRH1 which may comprise SEQ ID NO:4 or a variant having one or two amino acid substitutions, deletions or insertions in SEQ ID NO:4; ii. a CDRH2 which may comprise SEQ ID NO:5 or SEQ ID NO:6 or a variant having one or two amino acid substitutions, deletions or insertions in SEQ ID NO:5 or SEQ ID NO:6; iii. a CDRH3 which may comprise SEQ ID NO:7 or a variant having one or two amino acid substitutions, deletions or insertions in SEQ ID NO:7, and; iv. a framework region which may be at least 75% identical to the framework region of SEQ ID NO:39;
b) i. a CDRH1 which may comprise SEQ ID NO:11 or a variant having one or two amino acid substitutions, deletions or insertions in SEQ ID NO:11; ii. a CDRH2 which may comprise SEQ ID NO:12 or a variant having one or two amino acid substitutions, deletions or insertions in SEQ ID NO:12; iii. a CDRH3 which may comprise SEQ ID NO:13 or a variant having one or two amino acid substitutions, deletions or insertions in SEQ ID NO:13, and; iv. a framework region which may be at least 75% identical to the framework region of SEQ ID NO:46;
c) i. a CDRH1 which may comprise SEQ ID NO:17 or a variant having one or two amino acid substitutions, deletions or insertions in SEQ ID NO:17; ii. a CDRH2 which may comprise SEQ ID NO:18 or a variant having one or two amino acid substitutions, deletions or insertions in SEQ ID NO:18; iii. a CDRH3 which may comprise SEQ ID NO:19 or a variant having one or two amino acid substitutions, deletions or insertions in SEQ ID NO:19, and; iv. a framework region which may be at least 75% identical to the framework region of SEQ ID NO:48;
d) i. a CDRH1 which may comprise SEQ ID NO:23 or a variant having one or two amino acid substitutions, deletions or insertions in SEQ ID NO:23; ii. a CDRH2 which may comprise SEQ ID NO:24 or a variant having one or two amino acid substitutions, deletions or insertions in SEQ ID NO:24; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:50;
e) i. a CDRH1 which may comprise SEQ ID NO:23 or a variant having one or two amino acid substitutions, deletions or insertions in SEQ ID NO:23; ii. a CDRH2 which may comprise SEQ ID NO:70 or a variant having one or two amino acid substitutions, deletions or insertions in SEQ ID NO:70; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:69;
f) i. a CDRH1 which may comprise SEQ ID NO:29 or a variant having one or two amino acid substitutions, deletions or insertions in SEQ ID NO:29; ii. a CDRH2 which may comprise SEQ ID NO:30 or a variant having one or two amino acid substitutions, deletions or insertions in SEQ ID NO:30; iii. a CDRH3 which may comprise SEQ ID NO:31 or a variant having one or two amino acid substitutions, deletions or insertions in SEQ ID NO:31, and; iv. a framework region which may be at least 75% identical to the framework region of SEQ ID NO:52, or;
g) i. a CDRH1 which may comprise SEQ ID NO:35 or a variant having one or two amino acid substitutions, deletions or insertions in SEQ ID NO:35; ii. a CDRH2 which may comprise SEQ ID NO:36 or a variant having one or two amino acid substitutions, deletions or insertions in SEQ ID NO:36; iii. a CDRH3 which may comprise SEQ ID NO:37 or a variant having one or two amino acid substitutions, deletions or insertions in SEQ ID NO:37, and; iv. a framework region which may be at least 75% identical to the framework region of SEQ ID NO:54.

In accordance with the present invention, the naked antibody may comprise a complementary light chain variable region.

Such complementary light chain variable region may be selected, for example, from the group consisting of:
a) i. a CDRL1 which may comprise SEQ ID NO:1 or a variant having one or two amino acid substitutions, deletions or insertions in SEQ ID NO:1; ii. a CDRL2 which may comprise SEQ ID NO:2 or a variant having one or two amino acid substitutions, deletions or insertions in SEQ ID NO:2; iii. a CDRL3 which may comprise SEQ ID NO:3 or a variant having one or two amino acid substitutions, deletions or insertions in SEQ ID NO:3, and; iv. a framework region which may be at least 75% identical to the framework region of SEQ ID NO:38;
b) i. a CDRL1 which may comprise SEQ ID NO:8 or a variant having one or two amino acid substitutions, deletions or insertions in SEQ ID NO:8; ii. a CDRL2 which may comprise SEQ ID NO:9 or a variant having one or two amino acid substitutions, deletions or insertions in SEQ ID NO:9; iii. a CDRL3 which may comprise SEQ ID NO:10 or a variant having one or two amino acid substitutions, deletions or insertions in SEQ ID NO:10, and; iv. a framework region which may be at least 75% identical to the framework region of SEQ ID NO:45;
c) i. a CDRL1 which may comprise SEQ ID NO:14 or a variant having one or two amino acid substitutions, deletions or insertions in SEQ ID NO:14; ii. a CDRL2 which may comprise SEQ ID NO:15 or a variant having one or two amino acid substitutions, deletions or insertions in SEQ ID NO:15; iii. a CDRL3 which may comprise SEQ ID NO:16 or a variant having one or two amino acid substitutions, deletions or insertions in SEQ ID NO:16, and; iv. a framework region which may be at least 75% identical to the framework region of SEQ ID NO:47;
d) i. a CDRL1 which may comprise SEQ ID NO:20 or a variant having one or two amino acid substitutions, deletions or insertions in SEQ ID NO:20; ii. a CDRL2 which may comprise SEQ ID NO:21 or a variant having one or two amino acid substitutions, deletions or insertions in SEQ ID NO:21; iii. a CDRL3 which may comprise SEQ ID NO:22 or a variant having one or two amino acid substitutions, deletions or insertions in SEQ ID NO:22, and; iv. a framework region which may be at least 75% identical to the framework region of SEQ ID NO:49;
e) i. a CDRL1 which may comprise SEQ ID NO:26 or a variant having one or two amino acid substitutions, deletions or insertions in SEQ ID NO:26; ii. a CDRL2 which may comprise SEQ ID NO:27 or a variant having one or two amino acid substitutions, deletions or insertions in SEQ ID NO:27; iii. a CDRL3 which may comprise SEQ ID NO:28 or a variant having one or two amino acid substitutions, deletions or insertions in SEQ ID NO:28, and; iv. a framework region which may be at least 75% identical to the framework region of SEQ ID NO:51, or;
f) i. a CDRL1 which may comprise SEQ ID NO:32 or a variant having one or two amino acid substitutions, deletions or insertions in SEQ ID NO:32; ii. a CDRL2 which may comprise SEQ ID NO:33 or a variant having one or two amino acid substitutions, deletions or insertions in SEQ ID NO:33; iii. a CDRL3 which may comprise SEQ ID NO:34 or a variant having one or two amino acid substitutions, deletions or insertions in SEQ ID NO:34, and; iv. a framework region which may be at least 75% identical to the framework region of SEQ ID NO:53.

In accordance with the present invention, the pharmaceutical composition may comprise a naked antibody which may be used in combination with a cytotoxic drug.

Also in accordance with the present invention, the pharmaceutical composition may comprise a naked antibody which may optionally be conjugated with a cytotoxic drug.

In additional aspects, the present invention relates to an antibody or an antigen binding fragment thereof, which may comprise a heavy chain variable region comprising:
a) i. a CDRH1 which may comprise SEQ ID NO:4 or a variant having one or two amino acid substitutions, deletions or insertions in SEQ ID NO:4; ii. a CDRH2 which may comprise SEQ ID NO:5 or SEQ ID NO:6 or a variant having one or two amino acid substitutions, deletions or insertions in SEQ ID NO:5 or SEQ ID NO:6; iii. a CDRH3 which may comprise SEQ ID NO:7 or a variant having one or two amino acid substitutions, deletions or insertions in SEQ ID NO:7, and; iv. a framework region which may have, for example, at least 71, 72, 73, etc. (consecutive) amino acids of the framework region of SEQ ID NO:39;
b) i. a CDRH1 which may comprise SEQ ID NO:11 or a variant having one or two amino acid substitutions, deletions or insertions in SEQ ID NO:11; ii. a CDRH2 which may comprise SEQ ID NO:12 or a variant having one or two amino acid substitutions, deletions or insertions in SEQ ID NO:12; iii. a CDRH3 which may comprise SEQ ID NO:13 or a variant having one or two amino acid substitutions, deletions or insertions in SEQ ID NO:13, and; iv. a framework region which may have, for example, at least 67, 68, 69, etc. (consecutive) amino acids of the framework region of SEQ ID NO:46;
c) i. a CDRH1 which may comprise SEQ ID NO:17 or a variant having one or two amino acid substitutions, deletions or insertions in SEQ ID NO:17; ii. a CDRH2 which may comprise SEQ ID NO:18 or a variant having one or two amino acid substitutions, deletions or insertions in SEQ ID NO:18; iii. a CDRH3 which may comprise SEQ ID NO:19 or a variant having one or two amino acid substitutions, deletions or insertions in SEQ ID NO:19, and; iv. a framework region which may have, for example, at least 63, 64, 65, etc. (consecutive) amino acids of the framework region of SEQ ID NO:48;
d) i. a CDRH1 which may comprise SEQ ID NO:23 or a variant having one or two amino acid substitutions, deletions or insertions in SEQ ID NO:23; ii. a CDRH2 which may comprise SEQ ID NO:24 or a variant having one or two amino acid substitutions, deletions or insertions in SEQ ID NO:24; iii. a CDRH3 which may comprise SEQ ID NO:25 or a variant having one or two amino acid substitutions, deletions or insertions in SEQ ID NO:25, and; iv. a framework region which may have, for example, at least 70, 71, 72, etc. (consecutive) amino acids of the framework region of SEQ ID NO:50;
e) i. a CDRH1 which may comprise SEQ ID NO:23 or a variant having one or two amino acid substitutions, deletions or insertions in SEQ ID NO:23; ii. a CDRH2 which may comprise SEQ ID NO:70 or a variant having one or two amino acid substitutions, deletions or insertions in SEQ ID NO:70; iii. a CDRH3 which may comprise SEQ ID NO:25 or a variant having one or two amino acid substitutions, deletions or insertions in SEQ ID NO:25, and; iv. a framework region which may be at least 75% identical to the framework region of SEQ ID NO:69;
f) i. a CDRH1 which may comprise SEQ ID NO:29 or a variant having one or two amino acid substitutions, deletions or insertions in SEQ ID NO:29; ii. a CDRH2 which may comprise SEQ ID NO:30 or a variant having one or two amino acid substitutions, deletions or insertions in SEQ ID NO:30; iii. a CDRH3 which may comprise SEQ ID NO:31 or a variant having one or two amino acid substitutions, deletions or insertions in SEQ ID NO:31, and; iv. a framework region which may have, for example, at least 72, 73, 74, etc. (consecutive) amino acids of the framework region of SEQ ID NO:52, or;
g) i. a CDRH1 which may comprise SEQ ID NO:35 or a variant having one or two amino acid substitutions, deletions or insertions in SEQ ID NO:35; ii. a CDRH2 which may comprise SEQ ID NO:36 or a variant having one or two amino acid substitutions, deletions or insertions in SEQ ID NO:36; iii. a CDRH3 which may comprise SEQ ID NO:37 or a variant having one or two amino acid substitutions, deletions or insertions in SEQ ID NO:37, and; iv. a framework region which may have, for example, at least 71, 72, 73, etc. (consecutive) amino acids of the framework region of SEQ ID NO:54.

Such antibody or antigen binding fragment may comprise a light chain variable region comprising, for example:
a) i. a CDRL1 which may comprise SEQ ID NO:1 or a variant having one or two amino acid substitutions, deletions or insertions in SEQ ID NO:1; ii. a CDRL2 which may comprise SEQ ID NO:2 or a variant having one or two amino acid substitutions, deletions or insertions in SEQ ID NO:2; iii. a CDRL3 which may comprise SEQ ID NO:3 or a variant having one or two amino acid substitutions, deletions or insertions in SEQ ID NO:3, and; iv. a framework region which may have, for example, at least 67, 68, 69, etc. (consecutive) amino acids of the framework region of SEQ ID NO:38;
b) i. a CDRL1 which may comprise SEQ ID NO:8 or a variant having one or two amino acid substitutions, deletions or insertions in SEQ ID NO:8; ii. a CDRL2 which may comprise SEQ ID NO:9 or a variant having one or two amino acid substitutions, deletions or insertions in SEQ ID NO:9; iii. a CDRL3 which may comprise SEQ ID NO:10 or a variant having one or two amino acid substitutions, deletions or insertions in SEQ ID NO:10, and; iv. a framework region which may have, for example, at least 67, 68, 69, etc. (consecutive) amino acids of the framework region of SEQ ID NO:45;
c) i. a CDRL1 which may comprise SEQ ID NO:14 or a variant having one or two amino acid substitutions, deletions or insertions in SEQ ID NO:14; ii. a CDRL2 which may comprise SEQ ID NO:15 or a variant having one or two amino acid substitutions, deletions or insertions in SEQ ID NO:15; iii. a CDRL3 which may comprise SEQ ID NO:16 or a variant having one or two amino acid substitutions, deletions or insertions in SEQ ID NO:16, and; iv. a framework region which may have, for example, at least 70, 71, 72, etc. (consecutive) amino acids of the framework region of SEQ ID NO:47;
d) i. a CDRL1 which may comprise SEQ ID NO:20 or a variant having one or two amino acid substitutions, deletions or insertions in SEQ ID NO:20; ii. a CDRL2 which may comprise SEQ ID NO:21 or a variant having one or two amino acid substitutions, deletions or insertions in SEQ ID NO:21; iii. a CDRL3 which may comprise SEQ ID NO:22 or a variant having one or two amino acid substitutions, deletions or insertions in SEQ ID NO:22, and; iv. a framework region which may have, for example, at least 66, 67, 68, etc. (consecutive) amino acids of the framework region of SEQ ID NO:49;
e) i. a CDRL1 which may comprise SEQ ID NO:26 or a variant having one or two amino acid substitutions, deletions or insertions in SEQ ID NO:26; ii. a CDRL2 which may comprise SEQ ID NO:27 or a variant having one or two amino acid substitutions, deletions or insertions in SEQ ID NO:27; iii. a CDRL3 which may comprise SEQ ID NO:28 or a variant having one or two amino acid substitutions, deletions or insertions in SEQ ID NO:28, and; iv. a framework region which may have, for example, at least 68, 67, 68, etc. (consecutive) amino acids of the framework region of SEQ ID NO:51, or;
f) i. a CDRL1 which may comprise SEQ ID NO:32 or a variant having one or two amino acid substitutions, deletions or insertions in SEQ ID NO:32; ii. a CDRL2 which may comprise SEQ ID NO:33 or a variant having one or two amino acid substitutions, deletions or insertions in SEQ ID NO:33; iii. a CDRL3 which may comprise SEQ ID NO:34 or a variant having one or two amino acid substitutions, deletions or insertions in SEQ ID NO:34, and; iv. a framework region which may have, for example, at least 66, 67, 68, etc. (consecutive) amino acids of the framework region of SEQ ID NO:53.

In an exemplary embodiment, the invention provides an antibody or an antigen binding fragment thereof, which may comprise a heavy chain variable region and a light chain variable region, where the heavy chain variable region may comprise: i. a CDRH1 which may comprise SEQ ID NO:4 or a variant having one or two amino acid substitutions, deletions or insertions in SEQ ID NO:4; ii. a CDRH2 which may comprise SEQ ID NO:5 or SEQ ID NO:6 or a variant having one or two amino acid substitutions, deletions or insertions in SEQ ID NO:5 or SEQ ID NO:6; iii. a CDRH3 which may comprise SEQ ID NO:7 or a variant having one or two amino acid substitutions, deletions or insertions in SEQ ID NO:7, and; iv. a framework region which may have, for example, at least 71, 72, 73, etc. (consecutive) amino acids of the framework region of SEQ ID NO:39; and where the light chain variable region may comprise: i. a CDRL1 which may comprise SEQ ID NO:1 or a variant having one or two amino acid substitutions, deletions or insertions in SEQ ID NO:1; ii. a CDRL2 which may comprise SEQ ID NO:2 or a variant having one or two amino acid substitutions, deletions or insertions in SEQ ID NO:2; iii. a CDRL3 which may comprise SEQ ID NO:3 or a variant having one or two amino acid substitutions, deletions or insertions in SEQ ID NO:3, and; iv. a framework region which may have, for example, at least 67, 68, 69, etc. (consecutive) amino acids of the framework region of SEQ ID NO:38.

In another exemplary embodiment, the invention provides an antibody or an antigen binding fragment thereof, which may comprise a heavy chain variable region and a light chain variable region, where the heavy chain variable region may comprise: i. a CDRH1 which may comprise SEQ ID NO:11 or a variant having one or two amino acid substitutions, deletions or insertions in SEQ ID NO:11; ii. a CDRH2 which may comprise SEQ ID NO:12 or a variant having one or two amino acid substitutions, deletions or insertions in SEQ ID NO:12; iii. a CDRH3 which may comprise SEQ ID NO:13 or a variant having one or two amino acid substitutions, deletions or insertions in SEQ ID NO:13, and; iv. a framework region which may have, for example, at least 67, 68, 69, etc. (consecutive) amino acids of the framework region of SEQ ID NO:46; and where the light chain variable region may comprise: i. a CDRL1 which may comprise SEQ ID NO:8 or a variant having one or two amino acid substitutions, deletions or insertions in SEQ ID NO:8; ii. a CDRL2 which may comprise SEQ ID NO:9 or a variant having one or two amino acid substitutions, deletions or insertions in SEQ ID NO:9; iii. a CDRL3 which may comprise SEQ ID NO:10 or a variant having one or two amino acid substitutions, deletions or insertions in SEQ ID NO:10, and; iv. a framework region which may have, for example, at least 67, 68, 69, etc. (consecutive) amino acids of the framework region of SEQ ID NO:45.

In yet another exemplary embodiment, the invention provides antibody or an antigen binding fragment thereof, comprising a heavy chain variable region and a light chain variable region, where the heavy chain variable region may comprise: i. a CDRH1 which may comprise SEQ ID NO:17 or a variant having one or two amino acid substitutions, deletions or insertions in SEQ ID NO:17; ii. a CDRH2 which may comprise SEQ ID NO:18 or a variant having one or two amino acid substitutions, deletions or insertions in SEQ ID NO:18; iii. a CDRH3 which may comprise SEQ ID NO:19 or a variant having one or two amino acid substitutions, deletions or insertions in SEQ ID NO:19, and; iv. a framework region which may have, for example, at least 63, 64, 65, etc. (consecutive) amino acids of the framework region of SEQ ID NO:48; and where the light chain variable region may comprise: i. a CDRL1 which may comprise SEQ ID NO:14 or a variant having one or two amino acid substitutions, deletions or insertions in SEQ ID NO:14; ii. a CDRL2 which may comprise SEQ ID NO:15 or a variant having one or two amino acid substitutions, deletions or insertions in SEQ ID NO:15; iii. a CDRL3 which may comprise SEQ ID NO:16 or a variant having one or two amino acid substitutions, deletions or insertions in SEQ ID NO:16, and; iv. a framework region which may have, for example, at least 70, 71, 72, etc. (consecutive) amino acids of the framework region of SEQ ID NO:47.

In a further exemplary embodiment, the invention provides an antibody or an antigen binding fragment thereof, comprising a heavy chain variable region and a light chain variable region, where the heavy chain variable region may comprise: i. a CDRH1 which may comprise SEQ ID NO:23 or a variant having one or two amino acid substitutions, deletions or insertions in SEQ ID NO:23; ii. a CDRH2 which may comprise SEQ ID NO:24 or a variant having one or two amino acid substitutions, deletions or insertions in SEQ ID NO:24; iii. a CDRH3 which may comprise SEQ ID NO:25 or a variant having one or two amino acid substitutions, deletions or insertions in SEQ ID NO:25, and; iv. a framework region which may have, for example, at least 70, 71, 72, etc. (consecutive) amino acids of the framework region of SEQ ID NO:50; and where the light chain variable region may comprise: i. a CDRL1 which may comprise SEQ ID NO:20 or a variant having one or two amino acid substitutions, deletions or insertions in SEQ ID NO:20; ii. a CDRL2 which may comprise SEQ ID NO:21 or a variant having one or two amino acid substitutions, deletions or insertions in SEQ ID NO:21; iii. a CDRL3 which may comprise SEQ ID NO:22 or a variant having one or two amino acid substitutions, deletions or insertions in SEQ ID NO:22, and; iv. a framework region which may have, for example, at least 66, 67, 68, etc. (consecutive) amino acids of the framework region of SEQ ID NO:49.

In another exemplary embodiment, the invention provides an antibody or an antigen binding fragment thereof, comprising a heavy chain variable region and a light chain variable region, where the heavy chain variable region may comprise: i. a CDRH1 which may comprise SEQ ID NO:23 or a variant having one or two amino acid substitutions, deletions or insertions in SEQ ID NO:23; ii. a CDRH2 which may comprise SEQ ID NO:70 or a variant having one or two amino acid substitutions, deletions or insertions in SEQ ID NO:70; iii. a CDRH3 which may comprise SEQ ID NO:25 or a variant having one or two amino acid substitutions, deletions or insertions in SEQ ID NO:25, and; iv. a framework region which may have, for example, at least 68, 69, 70, etc. (consecutive) amino acids of the framework region of SEQ ID NO:69; and where the light chain variable region may comprise: i. a CDRL1 which may comprise SEQ ID NO:20 or a variant having one or two amino acid substitutions, deletions or insertions in SEQ ID NO:20; ii. a CDRL2 which may comprise SEQ ID NO:21 or a variant having one or two amino acid substitutions, deletions or insertions in SEQ ID NO:21; iii. a CDRL3 which may comprise SEQ ID NO:22 or a variant having one or two amino acid substitutions, deletions or insertions in SEQ ID NO:22, and; iv. a framework region which may have, for example, at least 66, 67, 68, etc. (consecutive) amino acids of the framework region of SEQ ID NO:49.

In yet a further exemplary embodiment, the invention provides antibody or an antigen binding fragment thereof, comprising a heavy chain variable region and a light chain variable region, where the heavy chain variable region may comprise: i. a CDRH1 which may comprise SEQ ID NO:29 or a variant having one or two amino acid substitutions, deletions or insertions in SEQ ID NO:29; ii. a CDRH2 which may comprise SEQ ID NO:30 or a variant having one or two amino acid substitutions, deletions or insertions in SEQ ID NO:30; iii. a CDRH3 which may comprise SEQ ID NO:31 or a variant having one or two amino acid substitutions, deletions or insertions in SEQ ID NO:31, and; iv. a framework region which may have, for example, at least 72, 73, 74, etc. (consecutive) amino acids of the framework region of SEQ ID NO:52, and where the light chain variable region may comprise: i. a CDRL1 which may comprise SEQ ID NO:26 or a variant having one or two amino acid substitutions, deletions or insertions in SEQ ID NO:26; ii. a CDRL2 which may comprise SEQ ID NO:27 or a variant having one or two amino acid substitutions, deletions or insertions in SEQ ID NO:27; iii. a CDRL3 which may comprise SEQ ID NO:28 or a variant having one or two amino acid substitutions, deletions or insertions in SEQ ID NO:28, and; iv. a framework region which may have, for example, at least 66, 67, 68, etc. (consecutive) amino acids of the framework region of SEQ ID NO:51.

In another exemplary embodiment, the invention provides antibody or an antigen binding fragment thereof, comprising a heavy chain variable region and a light chain variable region, where the heavy chain variable region may comprise: i. a CDRH1 which may comprise SEQ ID NO:35 or a variant having one or two amino acid substitutions, deletions or insertions in SEQ ID NO:35; ii. a CDRH2 which may comprise SEQ ID NO:36 or a variant having one or two amino acid substitutions, deletions or insertions in SEQ ID NO:36; iii. a CDRH3 which may comprise SEQ ID NO:37 or a variant having one or two amino acid substitutions, deletions or insertions in SEQ ID NO:37, and; iv. a framework region which may have, for example, at least 71, 72, 73, etc. (consecutive) amino acids of the framework region of SEQ ID NO:54 and where the light chain variable region may comprise: i. a CDRL1 which may comprise SEQ ID NO:32 or a variant having one or two amino acid substitutions, deletions or insertions in SEQ ID NO:32; ii. a CDRL2 which may comprise SEQ ID NO:33 or a variant having one or two amino acid substitutions, deletions or insertions in SEQ ID NO:33; iii. a CDRL3 which may comprise SEQ ID NO:34 or a variant having one or two amino acid substitutions, deletions or insertions in SEQ ID NO:34, and; iv. a framework region which may have, for example, at least 66, 67, 68, etc. (consecutive) amino acids of the framework region of SEQ ID NO:53.

In accordance with the present invention, the antibody may comprise two light chains and two heavy chains. The present invention relates to an antigen binding fragment obtained from any of the antibody described herein.

In another aspect, the present invention relates to the use of the antibody or antigen binding fragment described herein for reducing the growth of a prostate cancer cell.

In yet another aspect, the present invention relates to the use of the antibody or antigen binding fragment described herein, in the preparation of a medicament for reducing the growth of a prostate cancer cell.

In an additional aspect, the present invention relates to a composition comprising the antibody or antigen binding fragment described herein and a carrier.

In yet an additional aspect, the present invention relates to a pharmaceutical composition comprising the antibody or antigen binding fragment described herein and a pharmaceutically acceptable carrier.

In accordance with the present invention, the pharmaceutical composition may further comprise a cytotoxic drug.

Also in accordance with the present invention, the antibody or antigen binding fragment may optionally be conjugated with a cytotoxic drug.

As used herein, “at least 95% identical” refers to 95% (or more, for example, 95.5%, 96%, 96.5%, 97%, 97.5%, 98%, 98.5%, 99%, 99.5%, 99.9%) sequence identity between two sequences. Therefore, any polypeptide having at least 95% identity with an original polypeptide which does not destroy significantly a desired activity, function or immunogenicity is encompassed herein. The non-identical amino acids may correspond for example, to non-conservative amino acid substitution but preferably to conservative amino acid substitutions.

In yet a further aspect the present invention relates to an isolated nucleic acid capable of encoding the polypeptide(s) described herein.

The present invention relates in an additional aspect thereof to a vector that may comprise the nucleic acid described herein.

Further scope, applicability and advantages of the present invention will become apparent from the non-restrictive detailed description given hereinafter. It should be understood, however, that this detailed description, while indicating exemplary embodiments of the invention, is given by way of example only, with reference to the accompanying drawings.

The following examples are presented to illustrate the invention but it is not to be considered as limited thereto.

### Example 1

### PSMA Antibodies Preparation

### Materials and Methods

Monoclonal antibodies against an extracellular epitope of PSMA were generated as described in international application No. PCT/CA2004/000127 filed in Jan. 28, 2004 in the name of Cuello et al. More particularly, PS0215 (SEQ ID NO.: 56) was synthesized by FMOC synthesis with >85% purity and included a cysteine residue at the amino terminus for conjugation to the sulfhydryl-reactive carrier protein keyhole limpet hemocyanin (KLH) or bovine serum albumin (BSA) using N-maleimide chemistry. The conjugated peptide was used to immunize animals. Hybridomas secreting monoclonal antibodies against the peptide were characterized and the reactivity and specificity of the monoclonal antibodies towards PSMA-expressing cells or towards PS0215 was confirmed. Recombinant PSMA, peptide and membranes of PSMA-expressing cells were also obtained in accordance with methods known in the art or as described in PCT/CA2004/000127.

Solid-phase ELISA: An ELISA assay was used to detect anti-PSMA antibodies in mouse serum and hybridoma supernatants and for testing the specificity of purified MAbs. Briefly, 96-well plates (Maxi-Sorp, Nalgene Nunc, Rochester, N.Y.) were coated overnight at 4° C. or for 2 h at 37° C. with 100 uL of PBS containing 5 ug of cell membrane preparation, or 5 ng of purified recombinant human PSMA or BSA or 500 ng of PS0215 peptide. Plates were washed four times with 200 uL of 10 mM Tris-HCl, 150 mM NaCl, and 0.05% Tween-20 (TBST, pH 7.5), and blocked for a minimum of 30 min with 200 uL of TBST containing 3% casein. Plates were then washed and incubated for 1 h at room temperature with gentle agitation using 100 uL of either the undiluted or diluted test sample in TBST. In some of the assays, the test sample was preincubated for 15 min with a dilution of the indicated peptide before being added to the wells. Antibody binding was detected by the sequential addition, followed by washing, of 100 uL of horseradish peroxidase (HRP) conjugated goat anti-mouse IgG whole molecule secondary antibody diluted 1:5000 in TBST, for 1 h at room temperature, and 100 uL of HRP colorimetric substrate solution 3,3′,5,5′-tetramethylbenzidine. The reaction was stopped with the addition of 100 uL of 0.5 M sulphuric acid and the absorbance was read at 450 nm in a microplate reader.

Purification of monoclonal antibodies: MAb-containing hybridoma supernatants were clarified by centrifugation at 3000×g, brought to a final concentration of 20 mM Tris-HCl (pH 7.5) and passed through a 0.45 μm filter before being loaded onto a HiTrap protein G HP column, according to the manufacturer's instructions (GE Healthcare Biosciences, Piscataway, N.J.). After washing, bound MAb was eluted using Immunopure Gentle Ag/Ab Elution buffer (Pierce). Fractions were collected and examined for protein content by monitoring the absorbance at 280 nm. Protein-containing fractions were pooled and subsequently dialyzed overnight against PBS at 4° C. The dialyzed protein solution was then concentrated to at least 1 mg/mL, supplemented with 10% glycerol and stored frozen at −20° C. The purity of each MAb was verified by Coomassie staining following SDS-PAGE.

### Example 2

### PSMA Antibodies Structure

Isotyping was determined using Isostrips (Roche Diagnostics Corp., Indianapolis Ind.) and was confirmed to be either IgG1 k or IgG3 k.

The nucleic acid and the amino acid sequence of the antigen binding fragment was determined. Total RNA from ten millions hybridoma cells was extracted using Trizol (Invitrogen) according to the manufacturer's recommendations. The resulting RNA was reverse-transcribed into cDNA with ThermoScript RT and oligo(dT) primers according to the manufactrer's protocol (Invitrogen). DNA corresponding to the IgG heavy or light chain was then amplified by PCR using the oligonucleotides pair 5′-TGAGGTGCAGCTGGAGGAGTC-3′ (SEQ ID NO: 57) and 5′-GTGACCGTGGTCCCTGCGCCCCAG-3′ (SEQ ID NO:58) or 5′-GACATTCTGATGACCCAGTCT-3′ (SEQ ID NO:59) and 5′-TTTTATTTCCAGCTTGGTCCC-3′ (SEQ ID NO:60) respectively. The resulting PCR product was cloned into plasmid pCR2.1 TOPO (Invitrogen). The insert DNA from selected recombinants was sequenced and an Ig reading frame identified. The complementarity determining regions (CDRL1, CDRL2, CDRL3 and CDRH1, CDRH2, CDRH3) in the antibody sequence were identified by analysing the sequence and following a set of rules based on the Kabat sequence definition, described in http://www.bioinf.org.uk. The sequences obtained for all six antibodies is shown in FIG. 1.

### Example 3

### PSMA Antibodies Characterization

Antibody binding assay: Saturation binding studies were performed on whole cells with purified anti-PSMA MAb followed by detection of cellbound MAb using ¹²⁵I-labeled goat anti-mouse IgG. Briefly, nearly confluent LNCaP cells were rinsed with ice-cold PBS and scraped in PBS containing the protease inhibitor cocktail described above. Cells (7.5×10⁶ per tube) were incubated with 100 μL of antibody diluted in complete RPMI to various concentrations for 1 h at room temperature. After washing, 100,000 dpm of ¹²⁵I-labeled goat anti-mouse IgG at a specific activity of 872 dpm/pmol was added to cells for 1 h at room temperature. Following removal of unbound secondary antibody by centrifugation, the radioactivity associated with the cell pellet was determined using a gamma counter. Non-specific binding was determined in the presence of a 100-fold molar excess of the antibody antigen PSMA₄₉₀₋₅₀₀. The average non-specific binding of all antibodies reached 26% of the total binding at the maximal primary antibody concentration. For these experiments, a parallel sample in which the primary MAb was replaced by diluent served as a control for background binding of the secondary antibody and was less than 0.5%. Counts were analyzed by non-linear regression of total binding (Y) according to the law of mass action using the formula Y=Bmax*X/(Kd+X), where Bmax, Kd, and X represent the maximal binding, the concentration of ligand at half maximal binding, and the concentration of primary antibody, respectively. The ¹²⁵I-labeled goat anti-mouse IgG used in these experiments was radio-iodinated using the chloramine T method. Briefly, 10 μg of goat anti-mouse IgG whole molecule was incubated with 300 pmol of chloramine T and 90 μCi of [¹²⁵I] sodium iodide in a final volume of 25 μL containing 0.5 M sodium phosphate (pH 7.5) for 20 min at room temperature before quenching the reaction with 100 μL of sodium metabisulfite at 2.6 mg/mL in 0.5 M sodium phosphate buffer (pH 7.5). The labeled antibody was purified from free iodide by gel filtration on Sephadex G25. The amount of free iodide contaminating the labeled antibody was evaluated by ITLC-SG and never exceeded 1%. The tested affinity of the antibodies is shown below in TABLE 2.

TABLE 2
Binding parameters of monoclonal antibodies

| Antibody | K_{d} (nM) ± SD | Bmax (pmol/mg) +/− SD |
|---|---|---|
| PSf34.1 | 11.0 ± 2.2 | 4.8 ± 0.2 |
| PSf42.1 | 14.7 ± 2.7 | 2.8 ± 0.1 |
| PSf42.2 | 11.0 ± 3.3 | 4.7 ± 0.2 |
| PSf42.3 | 20.4 ± 6.0 | 4.9 ± 0.3 |
| PSf42.4 | 34.1 ± 6.2 | 5.1 ± 0.3 |
| PSf47.1 | 11.0 ± 3.5 | 4.6 ± 0.2 |

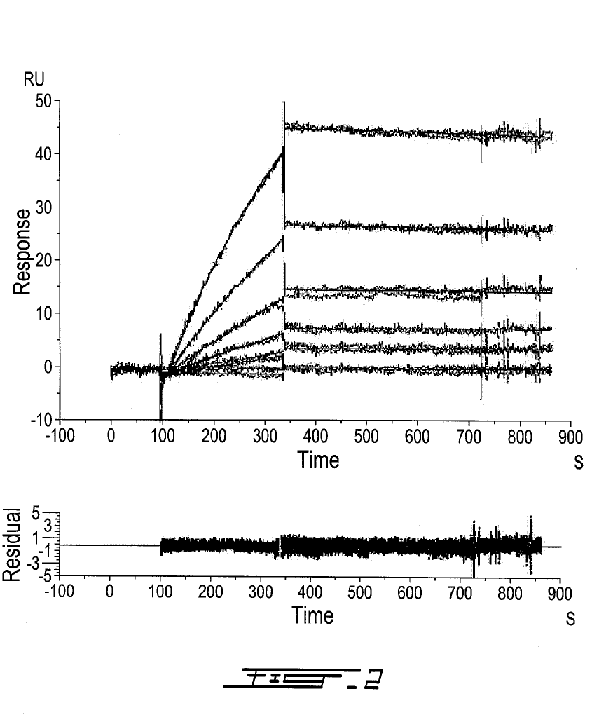

Surface plasmon resonance assay: Interaction kinetics between Mab PSf42.2 and PSMA was measured by surface plasmon resonance at 25° C. using a Biacore 3000 optical biosensor. PSMA was covalently immobilized to the surface of a CM5 sensor chip using standard amine coupling chemistry as previously described (De Crescenzo G et al. J Biol Chem 2001). Briefly, activation of the chip was performed by injecting an equimolar solution of N-ethyl-N′-(3-dimethyl aminopropyl)-carbodiimide hydrochloride and N-hydroxysuccinimide at a flow rate of 5 μL/min for 10 min. PSMA diluted in 10 mM acetate buffer (pH 5.0) was manually injected until 400 resonance units (RU) of protein was coupled. The remaining activated groups were then deactivated by injecting ethanolamine for 10 min. In addition to PSMA, a mock surface was generated using the same protocol in which PSMA injection was replaced by buffer injection. Following PSMA and mock surface preparation, the sensor chip surface and fluidic cartridge of the instrument were rinsed extensively with degassed running buffer (PBS containing 0.005% Tween-20). Various dilutions of antibodies (0, 18.75, 37.5, 75, 150 and 300 nM) and running buffer (9-300 nM, triplicates) were then injected at a flow rate of 30 μL/min for 4 min. Each injection was followed by injection of buffer for 6 min in order to record the dissociation of the PSMA-PSf42.2 complexes. Both complex formation and dissociation were recorded in real time (data collection frequency of 10 Hz). Sensor chip surfaces were regenerated between injections using three pulses (20 s each) of 10 mM glycine (pH 3.0) followed by an extraclean procedure in order to elute surface-bound antibody. Sensorgrams were then control-corrected using the double-referencing method prior to global fit analysis using the BIAevaluation 3.1 software package (Biacore) with a simple binding model. FIG. 2 show a control-corrected sensorgram related to PSf42.2 injections over PSMA surfaces. Related kinetic and themodynamic parameters determined by globally fitting the recorded sensorgrams with a simple Langmuirian model (A+B gives AB) are listed in TABLE 3. It was determined that the apparent affinity for PSf42.2 was 6.0±0.1 nM, and that the antibody-antigen complex was highly stable with a dissociation rate of koff of 0.646±0.01 10⁻⁴ s⁻¹. Interestingly, both affinity measurement methods, employing SPR and ¹²⁵I-labeled goat anti-mouse IgG secondary antibody, were in good agreement (6.0±0.1 nM vs. 11.0±3.3 nM).

TABLE 3
Kinetic and Thermodynamic parameter determined for PSf42.2
binding to immobilized PSMA

| | PSf42.2 |
|---|---|
| Kₒₙ (M⁻¹s⁻¹) | (1.07 ± 0.01) 10⁴ |
| K_{off} (s⁻¹) | (0.646 ± 0.01) 10⁻⁴ |
| Rmax (maximal binding | 84.4 ± 0.1 |
| capacity in RU) | |
| x² | 0.273 |
| K_{D} (nM) | 6.0 ± 0.1 |

### Example 4

### Tissue Reactivity of PSMA Antibodies

Tissue and tumor specificity of mAbs: to compare the tissue and tumor specificity of monoclonal antibodies of the present invention, tissue microarrays of 30 different normal tissues (Biochain) and 22 different tumor types (Dako) including prostate cancer was used in order to characterize their recognition specificity. The immunohistochemistry was performed on antigen retrieved, formalin fixed, paraffin embedded material. Anti-PSMA mAbs J591 (ATCC HB-12126) and 7E11 (ATCC HB-10494) were used as references. Formalin fixed paraffin embedded 5 μM sections were subjected to antigen retrieval in basic antigen retrieval solution (BD Pharmingen pH 9.5) in a microwavable pressure cooker for 10 min, cooled and equilibrated to 0.01M phosphate buffered saline (PBS) pH 7.4. Staining was carried out at room temperature in a humidified chamber. Endogenous peroxidases were inactivated with a 1% solution of H₂O₂ for 20 min, blocked with 5% normal goat serum (NGS) for 30 min and incubated with primary monoclonal antibodies (mAbs) diluted in PBS; 2% NGS overnight. Antibody binding to tissue sections was detected by the sequential addition of the following reagents followed by washing in PBS: goat anti-mouse IgG (H+L) (ICN) secondary antibody diluted 1:100 in PBS; 2% NGS for 1 hr, a complex of a bi-specific mAb mouse anti-peroxidase and horse radish peroxidase for 1 hr, and 3,3-diaminobenzidene tetrahydrochloride (DAB) at 0.6 mg/ml in PBS; 2% NGS; 0.01% H₂O₂ as chromogen. The primary antibodies were purified mAbs used at concentrations optimized in dilution experiments. PSf42.4 and PSf42.2 antibodies were used at 0.3 μg/ml, PSf42.1 at 0.16 ug/ml, PSf34.1 and PSf47.1 at 0.08 μg/ml, and J591 at 4 μg/ml. Mouse IgG mAb was used at a concentration of 0.2 μg/ml and served as a negative control for the primary antibodies. Tissue samples included organ confined and metastatic prostate cancer, HGPIN and normal prostate tissues drawn from radical prostatectomies, transurethral resections and autopsy material.

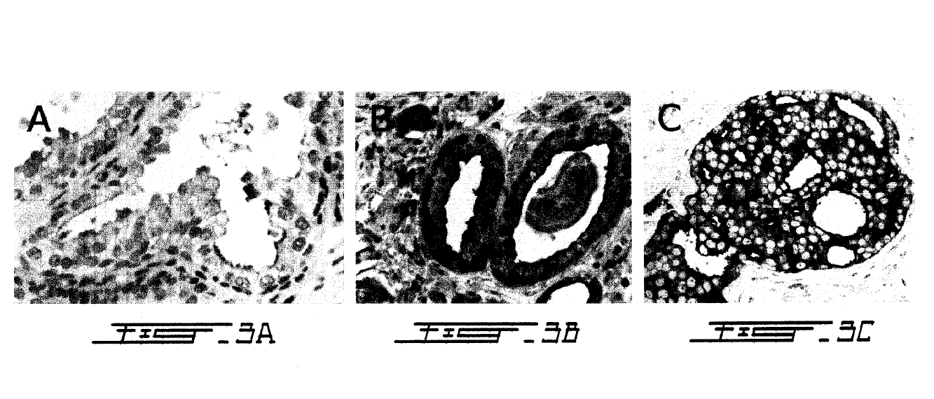

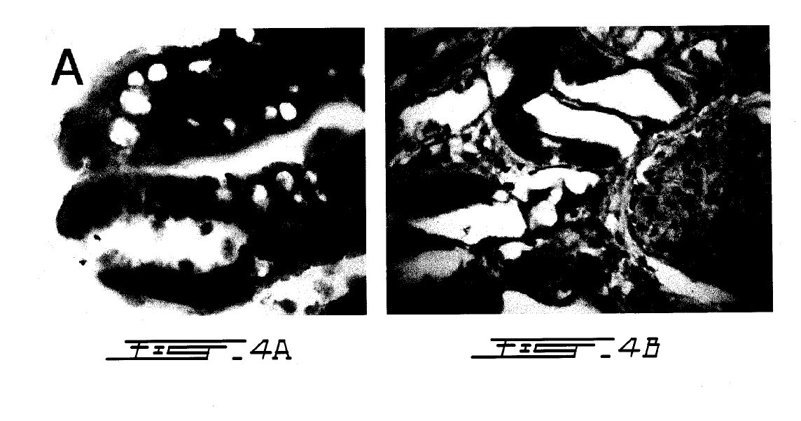

The tissue and tumor specificities of monoclonal antibodies of the present invention are shown in TABLE 4 and TABLE 5. mAbs showed the predicted tissue and tumor immunoreactivity and compared favorably with reference antibody J591, and PSf47.1 mAb achieved superior tissue specificity. FIG. 3 shows the predicted immunoreactivity to the apical surface of prostatic acinar cells in benign, premalignant and malignant prostatic tissues with more intense staining of cancer sections compared to benign. PSf42.2 stained benign prostatic glands with membranous reactivity at the luminal or apical surface of secretory cells (FIG. 3A). Immunoreactivity was upregulated in malignant prostatic glands with expression across various histologic grades (FIG. 3B; Gleason score 3+3=6 (PSf42.2) and FIG. 3C; Gleason score 4+4=8 (PSf47.1)). Moreover, protein expression was maintained in metastatic and hormone refractory cancers. These antibodies may therefore be useful in detecting hormone naive metastases as well as disease relapse once patients become hormone refractory. FIG. 4 shows immunoreactivity of PSf47.1 against small bowel (FIG. 4A) and proximal renal tubules (FIG. 4B).

TABLE 4
Tissue specificities of monoclonal PSMA antibodies

| | No. of positive cases/total no. of cases studied | | | | | | | |
|---|---|---|---|---|---|---|---|---|
| Tissue | PSf34.1 | PSf47.1 | PSf42.3 | PSf42.4 | PSf42.2 | PSf42.1 | J591 | IgG |
| Adipose | 0/2 | 0/2 | 0/2 | 0/2 | 0/2 | 0/2 | 0/2 | 0/2 |
| Bladder | 0/2 | 0/2 | 0/2 | 0/2 | 0/2 | 0/2 | 0/2 | 0/2 |
| Brain | 0/2 | 0/2 | 0/2 | 0/2 | 0/2 | 0/2 | 0/2 | 0/2 |
| Breast | 0/2 | 0/2 | 0/2 | 0/2 | 0/2 | 0/2 | 0/2 | 0/2 |
| Cerebullum | 0/2 | 0/2 | 0/2 | 0/2 | 0/2 | 0/2 | 0/2 | 0/2 |
| Cervix | 0/2 | 0/2 | 0/2 | 0/2 | 0/2 | 0/2 | 0/2 | 0/2 |
| Colon | 0/2 | 0/2 | 0/2 | 0/2 | 0/2 | 0/2 | 0/2 | 0/2 |
| Diaphragm | 2/2 | 0/2 | 2/2 | 2/2 | 2/2 | 2/2 | 2/2 | 0/2 |
| Duodenum | 1/2 | 2/2 | 2/2 | 0/2 | 2/2 | 0/2 | 0/2 | 0/2 |
| Esophagus | 0/2 | 0/2 | 2/2 | 0/2 | 0/2 | 0/2 | 0/2 | 0/2 |
| Gallbladder | 0/2 | 0/2 | 0/2 | 0/2 | 0/2 | 0/2 | 0/2 | 0/2 |
| Heart | 0/2 | 0/2 | 0/2 | 0/2 | 0/2 | 0/2 | 0/2 | 0/2 |
| Ileum | 2/2 | 0/2 | 2/2 | 2/2 | 0/2 | 0/2 | 2/2 | 0/2 |
| Jejenum | 2/2 | 2/2 | 2/2 | 2/2 | 2/2 | 0/2 | 2/2 | 0/2 |
| Kidney | 1/2 | 0/2 | 2/2 | 1/2 | 2/2 | 0/2 | 2/2 | 0/2 |
| Liver | 0/2 | 0/2 | 0/2 | 0/2 | 0/2 | 0/2 | 0/2 | 0/2 |
| Lung | 0/2 | 0/2 | 0/2 | 0/2 | 0/2 | 0/2 | 0/2 | 0/2 |
| Ovary | 0/2 | 0/2 | 0/2 | 0/2 | 0/2 | 0/2 | 0/2 | 0/2 |
| Pancreas | 0/2 | 0/2 | 0/2 | 0/2 | 0/2 | 0/2 | 0/2 | 0/2 |
| Placenta | 0/4 | 0/4 | 1/4 | 1/4 | 2/4 | 0/4 | 0/4 | 0/4 |
| Rectum | 0/2 | 0/2 | 0/2 | 0/2 | 0/2 | 0/2 | 0/2 | 0/2 |
| Skeletal muscle | 2/2 | 0/2 | 2/2 | 2/2 | 2/2 | 2/2 | 2/2 | 0/2 |
| Skin | 0/2 | 0/2 | 0/2 | 0/2 | 0/2 | 0/2 | 0/2 | 0/2 |
| Spleen | 2/2 | 1/2 | 0/2 | 0/2 | 0/2 | 0/2 | 0/2 | 0/2 |
| Stomach | 0/2 | 0/2 | 0/2 | 0/2 | 0/2 | 0/2 | 0/2 | 0/2 |
| Testis | 0/2 | 0/2 | 0/2 | 0/2 | 0/2 | 0/2 | 0/2 | 0/2 |
| Thymus | 0/2 | 0/2 | 0/2 | 0/2 | 0/2 | 0/2 | 0/2 | 0/2 |
| Thyroid | 0/2 | 0/2 | 0/2 | 0/2 | 0/2 | 0/2 | 0/2 | 0/2 |
| Tonsil | 0/2 | 0/2 | 0/2 | 0/2 | 0/2 | 0/2 | 0/2 | 0/2 |
| Uterus | 0/2 | 0/2 | 0/2 | 0/2 | 0/2 | 0/2 | 0/2 | 0/2 |

TABLE 5
Tumor specificities of monoclonal PSMA antibodies

| | No. of positive cases/total no. of cases studied | | | | | | | | |
|---|---|---|---|---|---|---|---|---|---|
| Tumor tissue | PSf34.1 | PSf47.1 | PSf42.3 | PSf42.4 | PSf42.2 | PSf42.1 | 7E11 | J591 | IgG |
| Astrocytoma | 1/2 | 0/2 | 0/2 | 0/2 | 0/2 | 0/2 | 0/2 | 0/2 | 0/2 |
| Breast carcinoma | 3/3 | 0/2 | 0/2 | 0/2 | 2/4 | 0/2 | 1/4 | 0/2 | 0/2 |
| Carcinoid | 2/2 | 0/3 | 0/3 | 0/3 | 0/3 | 0/3 | 1/3 | 0/3 | 0/3 |
| Colonic adenocarcinoma | 0/2 | 0/2 | 0/2 | 0/2 | 0/2 | 0/2 | 0/2 | 0/2 | 0/2 |
| Epithelioid sarcoma | 0/1 | 0/1 | 0/1 | 0/1 | 0/1 | 0/1 | 0/1 | 0/1 | 0/1 |
| Ewing's sarcoma | 0/1 | 0/1 | 0/1 | 0/1 | 0/1 | 0/1 | 0/1 | 0/1 | 0/1 |
| Gastric carcinoma | 0/2 | 0/2 | 0/2 | 0/2 | 2/2 | 0/2 | 1/2 | 0/2 | 0/2 |
| Hepatocellular carcinoma | 2/2 | 0/2 | 0/2 | 0/2 | 1/2 | 0/2 | 0/2 | 0/2 | 0/2 |
| Hodgkin's lymphoma | 7/7 | 0/13 | 2/13 | 5/13 | 6/13 | 1/13 | 4/12 | 1/13 | 0/13 |
| Leiomyoma | 1/2 | 0/2 | 0/2 | 0/2 | 0/2 | 0/2 | 0/2 | 0/2 | 0/2 |
| Lung adeno carcinoma | 2/2 | 0/2 | 0/2 | 0/2 | 0/2 | 0/2 | 0/2 | 0/2 | 0/2 |
| Lymphoma | 1/1 | 0/3 | 0/3 | 0/3 | 0/2 | 0/3 | 0/3 | 0/3 | 0/3 |
| Malignant fibrous histiocytoma | 0/0 | 0/1 | 0/1 | 0/1 | 0/1 | 0/1 | 0/0 | 0/1 | 0/1 |
| Melanoma | 2/3 | 0/4 | 2/4 | 2/4 | 2/4 | 0/3 | 2/3 | 0/4 | 0/4 |
| Mesothelioma | 0/2 | 0/2 | 0/2 | 0/2 | 0/2 | 0/2 | 0/2 | 0/2 | 0/2 |
| Ovarian carcinoma | 1/2 | 0/2 | 0/2 | 0/2 | 0/2 | 0/2 | 0/2 | 0/2 | 0/2 |
| Pancreatic carcinoma | 0/2 | 0/2 | 0/2 | 0/2 | 0/2 | 0/2 | 0/2 | 0/2 | 0/2 |
| Prostatic carcinoma | 2/2 | 1/1 | 1/1 | 1/1 | 1/1 | 1/1 | 1/1 | 1/1 | 0/1 |
| Renal cell carcinoma | 0/2 | 0/2 | 0/2 | 0/2 | 0/2 | 0/2 | 0/2 | 0/2 | 0/2 |
| Rhabdosarcoma | 2/2 | 0/2 | 2/2 | 2/2 | 1/1 | 0/2 | 2/2 | 0/2 | 0/2 |
| Thyroid carcinoma | 1/2 | 0/2 | 0/2 | 0/2 | 1/2 | 0/2 | 2/2 | 0/2 | 0/2 |
| Undifferentiated carcinoma | 0/4 | 0/4 | 0/4 | 0/4 | 0/4 | 0/4 | 0/4 | 0/4 | 0/4 |

### Example 5

### Prostate Cancer Detection

The mouse model used in the present experiment involved grafting LNCaP or PC-3 cells into male CD-1 nu/nu mice. Mice of at least 2 months old were injected subcutaneously in the rear tight with 100 ul of 0.5-2×10⁶ cells in a 50:50 solution of PBS and Matrigel (Becton Dickinson). Mouse was left to rest with water and food ad libitum until visible tumor developed. At that stage, the tumor was measured using a calliper to follow its development. The formula 4/3π(L/2)(I/2)², where L=length and I=width, was used to calculate the volume of the tumor.

Antibody conjugation to DOTA: Purified monoclonal antibodies and all solutions were treated with the chelating resin Chelex (Bio-Rad) to remove trace metal ions from samples and buffers. Antibody was washed in 0.1M sodium phosphate buffer (pH 8.2) and concentrated to 3 mg/ml (30 000 MWCO Microcon; Millipore). Then, 50× molar excess of 1,4,7,10-Tetraazacyclododecane-1,4,7,10-tetraacetic acid mono(N-hydroxysuccinimide ester) (DOTA-NHS ester) in dimethylformamide (DMF) was added to the concentrated antibody preparation and the reaction mixture was incubated for 30 minutes at room temperature. The resultant antibody-DOTA conjugate was separated from the excess unreacted DOTA-NHS ester by repeated washing with 0.3M ammonium acetate buffer (pH 6.5) (30 000 MWCO Microcon; Millipore).

¹¹¹In labelling of DOTA conjugate: Radiolabeling of the Ab-DOTA with In¹¹¹ was achieved by incubating 1 mCi of ¹¹¹InCl₃ (MDS-Nordion) per 1 mg of Ab-DOTA for 1 h at 43° C. Then, the antibody was washed in PBS (pH 7.5) (Amicon Ultra 15) to remove the unchelated free ¹¹¹In. The purity of the resulting Ab-DOTA-In¹¹¹ was determined by instant thin layer chromatography (ITLC-SG). A small portion (3 ul) of the radiolabeled product was spotted on a ITLC strip and the species were separated using a mobile phase consisting of a 1% solution of diethylene triamine pentaacetic acid (DTPA). Once the solvent front had reached an Rf value of approximately 0.9, the strip was removed from the mobile phase and cut in four equal portions, the bottom portion containing the Ab-DOTA with In¹¹¹ and the upper ones the free In¹¹¹. The strip portions were counted in a gamma counter in order to calculate the radio-purity and the specific activity of the conjugate. The radiopurity of the conjugate was calculated as 100× (cpm of bottom strip portion)/(total cpm of all strips), and reached >90%. The specific activity was calculated as the amount of radioactivity/quantity of protein, and reached 0.2-1 uCi/ug.

In vivo biodistribution of Ab-DOTA-In¹¹¹ by scintigraphy: mice with visible tumor were administered, by tail vein injection, 20-100 ug of radiolabeled antibody, Ab-DOTA-In¹¹¹, or an equivalent amount of radioactivity of free ¹¹¹InCl₃. At various times subsequent to the injection, mice were anaesthetized and the distribution of radioactivity was determined by scintigraphy of the whole mouse body. Image was acquired using a General Electric Millenium MG nuclear gamma camera from the ventral surface of the mouse body.

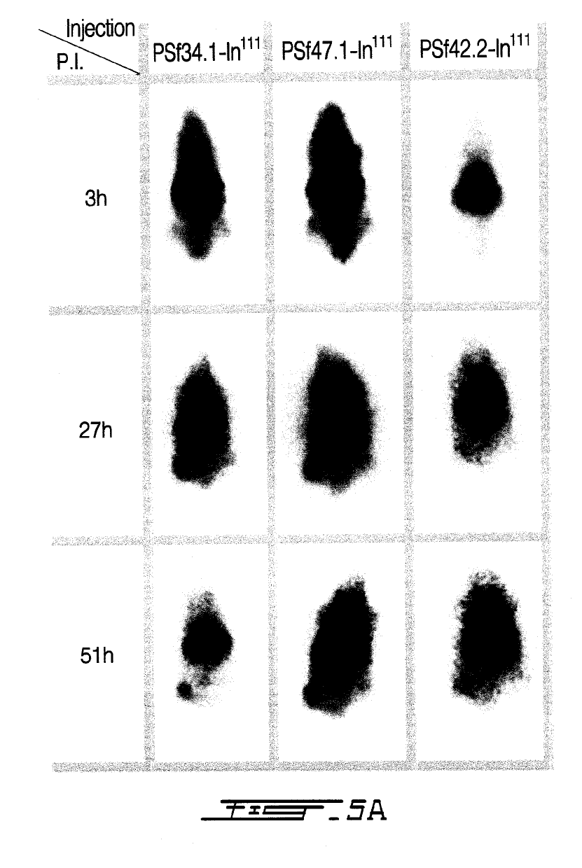

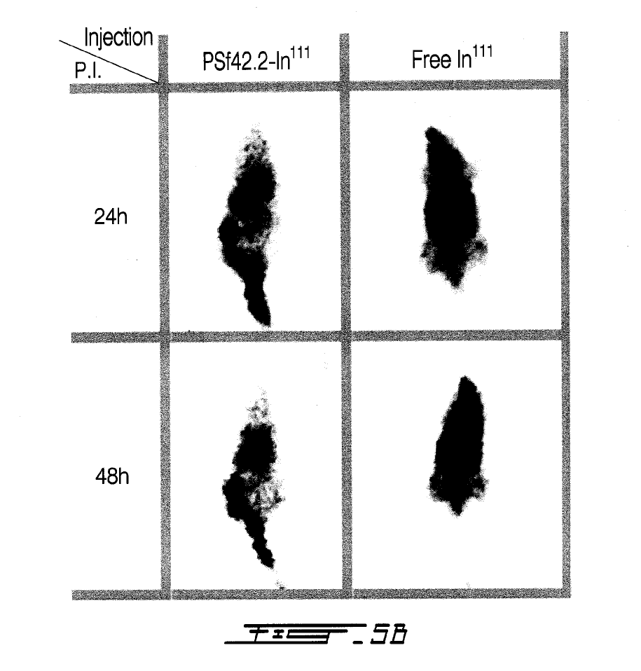

Scintigraphy was done 3 h, 27 h and 51 h post-injection. FIGS. 5A and B show the result of scintigraphy experiments using PSf34.1, PSf47.1 and PSf42.2 antibodies respectively.

### Example 6

### Treatment of Prostate Cancer Mouse Model with Antibody

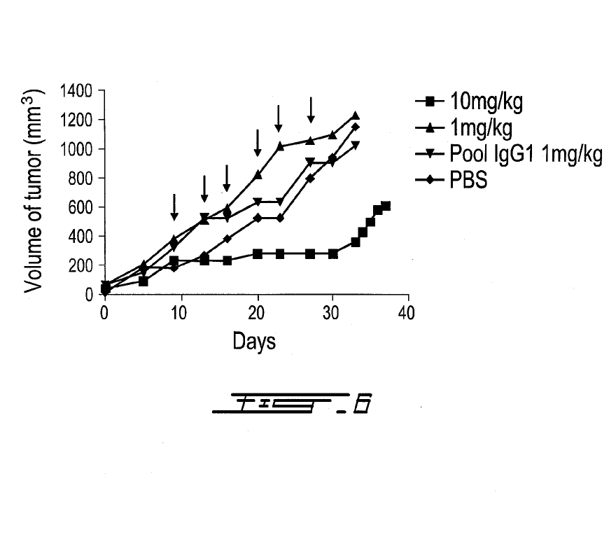

Mice bearing tumors with a continuous growing rate were selected for treatment and randomized. More specifically, mice bearing LNCaP tumor of 230, 381, 179, and 318 mm³ in volume were injected intravenously with PSf42.2-10 mg/kg, PSf42.2-1 mg/kg, IgG-1 mg/kg and PBS, respectively. Mouse was administered 100 ul of antibody solution (1 or 10 mg of antibody/kg of body weight) in PBS or PBS alone by intravenous injection in the tail vein. The treatment was repeated every 3-4 days. Five more injections over the next 18 days were administered to the animals in the same manner. The size of tumor was measured the day of the injection or at the same frequency after the treatment had cessed. FIG. 6 shows the graph of tumors volume before, during and after the treatment period. Mouse treated with PSf42.2-1 mg/kg, IgG-1 mg/kg or PBS had a similar, linear growing rate until the last day of the experiment. Indeed, at day 33, the tumors reached a volume of 1227, 1022 and 1150 mm³, respectively. In contrast, the tumor growth of the mouse treated with a higher dose of PSf42.2-10 mg/kg, was abrogated to 230 mm³ from the first injection and never exceeded 280 mm³ over the 18 days period of treatment. The animal did not suffer from apparent side effects, no weight lost, and the treatment was thus considered safe. The data shows that the growth of prostate tumor in prostate cancer animal model is susceptible to treatment with PSf42.2 in a dose-dependent manner. This effect is related to PSMA expressing tumor since the interruption of treatment reversed the growth inhibition of the tumor. Moreover, the specificity of the compound is further highlighted by the lack of apparent side effects, such as weight lost of the animal, during the course of the 18 days of treatment or until the animal was sacrificed at day 35. Overall, those results indicate that the anti-PSMA antibody has a role on the tumor growth control mechanism.

### Example 7

### PSMA Internalization

Cell surface biotinylation and PSMA internalization assay: the ability of membrane proteins to internalize is critical for the targeted delivery of cytotoxic compounds into cells. PSMA harbors a cytoplasmic N-terminal MXaaXaaXaaL motif (Xaa is any amino acid), which facilitates internalization via clathrin coated pits. We first tested whether PSMA undergoes PSf42.2-mediated internalization using a thiol-cleavable cell surface biotinylation assay. LNCaP cells were seeded at 1×10⁶ per 60 mm cell culture Petri dish 1 day prior to use. On the day of the experiment, cells were washed once with ice-cold PBS (pH 8.0) containing 1 mM CaCl₂ and 1 mM MgCl₂ before biotinylation of cell surface proteins using 2 mL of thiol-cleavable amine-reactive EZ-Link sulfo-NHS-SS-Biotin at a concentration of 0.5 mg/mL in PBS (pH 8.0) for 20 min at 4° C. Biotinylated cells were then incubated at 37° C. for 10, 20, or 60 min in RPMI medium or RPMI medium containing 1 μg/mL of purified MAb. Following incubation, cells were washed with ice-cold PBS containing 10% FBS and placed on ice to prevent further endocytosis. Cell-surface biotin was then cleaved by treating cells with PBS containing 2% FBS and 50 mM dithiothreitol (DTT) for 40 min on ice. As a control for these experiments, two dishes containing complete RPMI only were left on ice for 60 min. Cells in one dish were treated to remove cell surface biotin while cells in the other dish were left untreated, thus serving as 0 and 100% biotinylation references, respectively. To stop the cleavage reaction, the DTT-containing solution was removed and replaced with a solution of PBS containing 2% FBS and 5 mg/mL iodoacetamide for 15 min on ice. To immunoprecipitate PSMA, cells were solubilized in 1 mL of ice-cold mRIPA buffer containing 50 mM Tris-HCl, 150 mM NaCl, 4 mM EDTA, 1% Triton X-100, 0.5% deoxycholate, and 0.1% SDS. Following microcentrifugation of the cell lysate at 13000×g to remove insoluble material, 2 μg of purified anti-PSMA MAb J591 was added to the clarified supernatant and the tube gently rotated for 1 h at 4° C. Anti-PSMA MAb J591 was precipitated by the addition of 40 μL of a 50% slurry of washed protein G Sepharose Fast Flow and incubated for 1 h at 4° C. with gentle rotation. Beads were then washed four times with mRIPA and resuspended in 50 μL of a non-reducing Laemmli buffer. Proteins in the resulting supernatants were subjected to SDS-PAGE and then electroblotted onto a PVDF membrane for subsequent Western blot analysis, where biotinylated PSMA was detected using streptavidin-HRP according to the manufacturer's instructions. The intensity of the bands was quantified using the Image J software package.

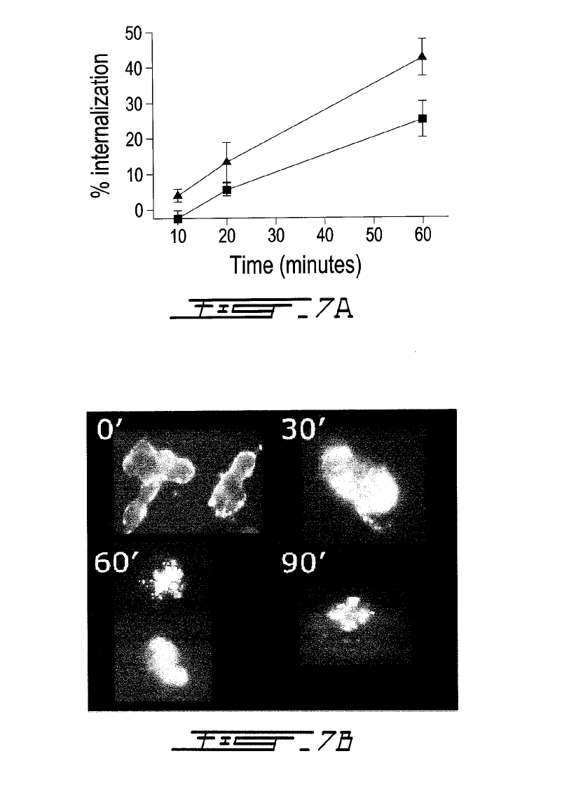

FIG. 7A shows the amount of internalized biotinylated PSMA quantified following incubation of LNCaP cells with anti-PSMA MAb or medium alone. After 60 min, a mean of 25.1±5.3% of total cell surface biotinylated PSMA was spontaneously internalized, while binding of PSf42.2 increased the amount of internalized PSMA to 42.5±5.5%. In FIG. 7B, internalization of PSMA was examined by immunofluorescence. Viable LNCaP cells were stained at 4° C. and then incubated at 37° C. in order to activate the internalization machinery and allow the PSMA-bound antibodies to internalize. At 4° C. (time 0), intense plasma membrane staining was revealed. Following incubation of the cells at 37° C., a gradual loss of staining at the plasma membrane was observed. This loss was concomitant with the detection of MAb in intracellular compartments. At the final time point, the MAb was localized in the perinuclear region of the cell. Taken together, these results indicate that anti-PSMA binding to LNCaP cells nearly doubles the rate of endogenous internalization of PSMA, and suggests that co-internalized PSf42.2 MAb is a suitable vehicle for the deliver of a cytosolic payload to PSMA-expressing cancer cells.

### Example 8

### PSMA Antibody Conjugation and Therapeutics

In vitro cytotoxicity of drug-conjugated antibody: the potential of PSf42.2 to deliver a cytotoxic payload to LNCaP cells was evaluated using conjugation methods based on Guillemard and Saragovi (2001), US2004/0115209 and US2006/0189515. A NHS-drug conjugate was prepared. The drugs (taxol, doxorubicin) were first succinylated. Taxol or doxorubicin, each freshly solubilized in DMF at a final concentration of 1 mg/ml, were mixed with a 50× molar excess of succinic anhydride in DMF and incubated at room temperature for 2 h (doxorubicin) or overnight (taxol). This reaction links a succinic acid molecule to taxol trough an ester bond at its C2′ position and to doxorubicin trough an ester or amide bond. Then, in a subsequent step, the newly available carboxylic acid formed from the succinylation of the drug is activated with a carbodiimide derivative and reacted with NHS to form a stable NHS-succinyl-drug conjugate. The crude succinylated drug from the first step is mixed with a 50× molar excess of an EDC solution at 100 mg/ml in DMSO, and incubated for 5 minutes at room temperature. Subsequently, a 50× molar excess of an NHS solution at 100 mg/ml in DMSO is added to the mixture and the incubation is pursued overnight at room temperature. The NHS-succinyl-drug conjugate is then purified by HPLC, lyophilised and stored under argon atmosphere at −20° C.

For conjugating the drug to the antibody, purified monoclonal antibody was first buffer exchanged to 100 mM Na Carbonate pH 8.6 and concentrated to 1-5 mg/ml (30 000 MWCO Microcon; Millipore). The antibody was then mixed with a 5× molar excess of the NHS-drug conjugate solubilized in DMF as such that the final percentage of DMF in the reaction mixture is below 10%. The reaction mixture is incubated for 20 minutes at room temperature and then the resulting antibody-drug conjugate was separated from the excess unreacted NHS-drug conjugate and buffered exchanged to PBS by gel filtration (Sephadex G25, Amersham). The ratio of bound doxorubicin was calculated by dividing the doxorubicin concentration (absorbance at 480 nm divided by the molar extinction coefficient; 11 500 cm-1 M-1) by the protein concentration. The measured molar ratio of mAb-bound doxorubicin was 1:2. The ratio of mAb-bound taxol was assumed to be the same as that for doxorubicin (i.e.: 1:2) as the conditions of conjugation and chemistry (amine reactive NHS-drug derivative) are the same.

The antibody was also conjugated to the ribosome-inactivating protein saporin, a RNA N-glycosidase purified from seeds of the plant Saponaria officinalis, trough a reduction sensitive linker. The antibody-saporin conjugate was made by Advanced Targeting Systems (San Diego, Calif.) and the mAb:saporin ratio of conjugation was 1.74 as determined by SDS-PAGE.

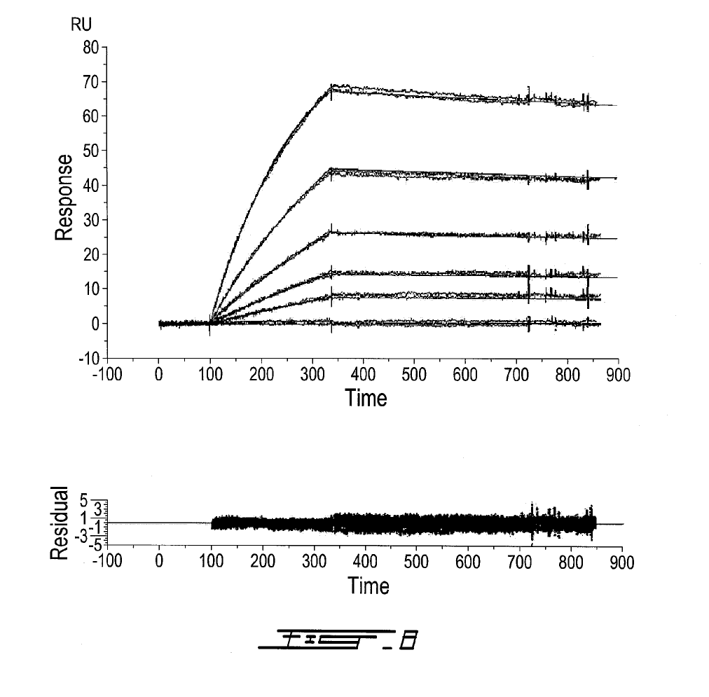

Surface plasmon resonance assay: Interaction kinetics between Mab PSf42.2-Taxol and PSMA was measured by surface plasmon resonance as described above. Results are shown in FIG. 8 and in TABLE 6. A close inspection of kinetic and themodynamic parameters reveals that there is no major differences between PSf42.2 and its taxol-conjugate for binding to PSMA (less than twofold differences for the kinetic constants). These differences in apparent kinetic constants may be attributable to the extremely weak dissociation rates whose precise determination would have required longer dissociation times (more than 2 hours for each sensorgram). Differences in association and dissociation rates do compensate for each other as indicated by the extreme similarity of both themodynamic values (TABLE 6).

TABLE 6
Kinetic and thermodynamic parameter determined for
Taxol-conjugated PSf42.2 binding to immobilized PSMA

| | Taxol-conjugated PSf42.2 |
|---|---|
| Kₒₙ (M⁻¹s⁻¹) | (1.96 ± 0.01) 10⁴ |
| K_{off} (s⁻¹) | (1.086 ± 0.01) 10⁻⁴ |
| Rmax (maximal binding | 90 ± 0.1 |
| capacity in RU) | |
| x² | 0.245 |
| K_{D} (nM) | 5.6 ± 0.1 |

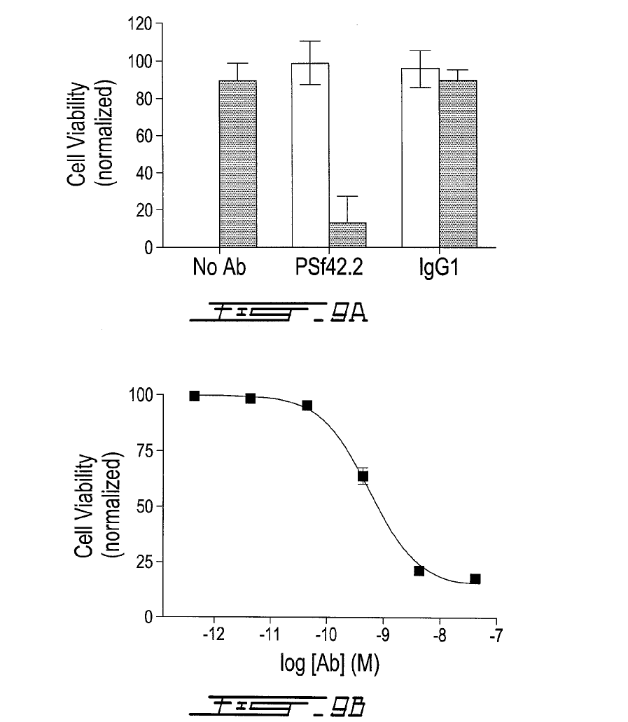

Cytotoxicity assay: the wells of 48 wells plate were each seeded with 40 000 LNCaP or 20 000 PC-3 and the cells were incubated for 24 hours under normal cell culture conditions (37° C., 5% CO₂ atmosphere). The following day, the media was replaced with 200 ul of cell culture media containing immunoconjugate (antibody-drug conjugate) at concentration ranging from 0.39 nM to 200 nM, or with an equimolar concentration of the unconjugated drug alone. After 3 days of incubation, the media was aspirated and the cells were fixed with a solution of 5% formalin in PBS for 15 minutes. The remaining live cells were stained by the crystal violet method. The fixation media was replaced with 250 ul of a solution of 0.2% crystal violet in 2% ethanol and the plate was incubated for 20 minutes at room temperature. The wells were then rinsed with tap water and the remaining crystal violet was solubilized in 200 ul of 1% acetic acid for 15 minutes. Its relative quantity was measured by dosage at 570 nm using a spectrophotometer. As shown in FIG. 9A, a dose-response of the immunoconjugates (IT) and an equivalent concentration of drug alone on the viability of LNCaP or PC-3 cells. The in vitro cytotoxic activity of the anti-PSMA monoclonal antibody in the form of an immunoconjugate was also evaluated using the antibody conjugated to saporin. FIG. 9B shows a dose-response of anti-PSMA-saporin immunoconjugate and saporin dose equivalent on prostate cancer cells viability, in vitro. The curves show that the LNCaP cells viability diminishes in a concentration-dependent manner following incubation with the immunoconjugate. At the lowest concentration tested (0.39 nM) the average cell viability was 60.3%. Curve fitting of those data points using a sigmoidal equation revealed an EC50 of 1 nM. In contrast, the same treatment did not significantly compromised the viability of PC-3 cells at any of the concentrations tested consistently with the fact that LNCaP cells expresses PSMA and not PC-3. Conjugating the antibody to a drug had an enhancing effect on the overall cytotoxic activity of the drug alone. The immunoconjugate was more potent at killing LNCaP cells that the equivalent concentration of the drug alone.

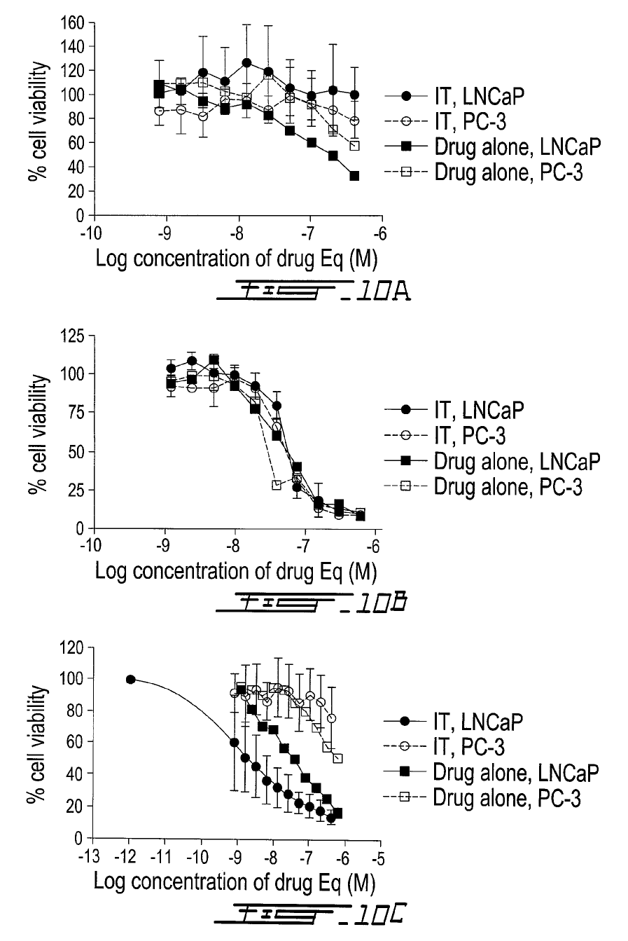

FIG. 10 shows a dose-response of anti-PSMA immunoconjugates on prostate cancer cells survival. Three immunoconjugates were constructed by conjugating the anti-PSMA antibody PSf42.2 to doxorubicin (A), paclitaxel (B), or saporin (C) as described above. The graphs show a dose-response of the three immunoconjugates (A, B and C respectively) and an equivalent concentration of drug alone on the viability of LNCaP or PC-3 cells. Overall, those results show that anti-PSMA can be used as an effective vehicle to deliver toxic drugs specifically to cells expressing PSMA.

Although the present invention has been described by way of exemplary embodiments, it should be understood by those skilled in the art that the foregoing and various other changes, omission and additions may be made therein and thereto, without departing from the spirit and scope of the present invention as defined in the appended claims.

### Exemplary Embodiments of Sequences Used in the Present Invention

SEQ ID NO.: 1
PSf34.1-CDRL1
KSSQSLLHSDGKTYLN
SEQ ID NO.: 2
PSf34.1-CDRL2
LVSRLDS
SEQ ID NO.: 3
PSf34.1-CDRL3
WQGTHFPRT
SEQ ID NO.: 4
PSf34.1-CDRH1
GFYIKDTYIH
SEQ ID NO.: 5
PSf34.1a-CDRH2
GIGSADGDTR
SEQ ID NO.: 6
PSf34.1-CDRH2
GIDPADGDTR
SEQ ID NO.: 7
PSf34.1-CDRH3
ELAY
SEQ ID NO.: 8
PSf42.1-CDRL1
KSSHSLLHRDGRTYLN
SEQ ID NO.: 9
PSf42.1-CDRL2
LVSKLDS
SEQ ID NO.: 10
PSf42.1-CDRL3
WQGTHFPRT
SEQ ID NO.: 11
PSf42.1-CDRH1
GLNIKDSYLH
SEQ ID NO.: 12
PSf42.1-CDRH2
GIDPANGDVE
SEQ ID NO.: 13
PSf42.1-CDRH3
FPY
SEQ ID NO.: 14
PSf42.2-CDRL1
RSSQSLVHSNGNTYLH
SEQ ID NO.: 15
PSf42.2-CDRL2
KASNRFS
SEQ ID NO.: 16
PSf42.2-CDRL3
FQSTHVPYT
SEQ ID NO.: 17
PSf42.2-CDRH1
GFNIKDTYMH
SEQ ID NO.: 18
PSf42.2-CDRH2
GIDPADGEPL
SEQ ID NO.: 19
PSf42.2-CDRH3
VRSSFDY
SEQ ID NO.: 20
PSf42.3-CDRL1
KSSQSLLHRDGKTYLN
SEQ ID NO.: 21
PSf42.3-CDRL2
LVSLVDS
SEQ ID NO.: 22
PSf42.3-CDRL3
WQGTHFPRT
SEQ ID NO.: 23
PSf42.3-CDRH1
GFNIKDTYMH
SEQ ID NO.: 24
PSf42.3-CDRH2
GIDPETGNTK
SEQ ID NO.: 25
PSf42.3-CDRH3
LGRPFAH
SEQ ID NO.: 26
PSf42.4-CDRL1
KSSHSLLHRDGRTYLN
SEQ ID NO.: 27
PSf42.4-CDRL2
LVSKLDS
SEQ ID NO.: 28
PSf42.4-CDRL3
WQGTHFPRT
SEQ ID NO.: 29
PSf42.4-CDRH1
GFSIRDTYMH
SEQ ID NO.: 30
PSf42.4-CDRH2
GIDPENGNSK
SEQ ID NO.: 31
PSf42.4-CDRH3
ELAY
SEQ ID NO.: 32
PSf47.1-CDRL1
KSSQSLLNSRTRKNYLA
SEQ ID NO.: 33
PSf47.1-CDRL2
WASTRES
SEQ ID NO.: 34
PSf47.1-CDRL3
KQSYNFIT
SEQ ID NO.: 35
PSf47.1-CDRH1
GYTFTVYVIH
SEQ ID NO.: 36
PSf47.1-CDRH2
YINPYNDGAE
SEQ ID NO.: 37
PSf47.1-CDRH3
GENYYTSRYGFFDV
SEQ ID NO.: 38
PSf34.1 light chain variable sequence
DILMTQSPLNLSVTIGQPASISCKSSQSLLHSDGKTYLNWLLQRPGQSPK
RLMYLVSRLDSGVPDRFTGSGSGTDFTLKISRVEAEDLGVYYCWQGTHFP
RTFGGGTKLEIKRA
SEQ ID NO.: 39
PSf34.1 heavy chain variable sequence
EVQLEESGAELVKPGASVKLSCTASGFYIKDTYIHWVKQRPEEVLEWIGG
IDPADGDTRYDPKFQGKATITADTSSNSAYLHLTSLTSEDTAVYFCAREL
AYWGAGTTVTVS
SEQ ID NO.: 40
PSf34.1a heavy chain variable sequence
EVQLEESGAELVKPGASVKLSCTASGFYIKDTYIHWVKQRPEEVLEWIGG
IGSADGDTRYDPKFQGKATITADTSSNSAYLHLTSLTSEDTAVYFCAREL
AYWGAG
SEQ ID NO.: 41
PSf34.1b heavy chain variable sequence
EVQLEESGAELVKPGASVKLSCTASGFYIKDTYIHWVKQRPEEVLEWIGG
IDPADGDTRYDPKFQGKATITADTSSNSAYLHLTSLTSEDTVVYFCAREL
AYWGAG
SEQ ID NO.: 42
PSf34.1c heavy chain variable sequence
EVQLEESGAELVKPGASVKLSCTASGFYIKDTYIHWVRQRPEEVLEWIGG
IDPADGDTRYDPKFQGKATITADTSSNSAYLHLTSLTSEDTAVYFCAREL
AYWGAGTTVT
SEQ ID NO.: 43
PSf34.1d heavy chain variable sequence
EVQLEESGAELVKPGASVKLSCTASGFYIKDTYIHWVKQRPEEVLEWIGG
IDPADGDTRYDPKSQGKATITADTSSNSAYLHLTSLTSEDTAVYFCAREL
AYWGAGTTVTIT
SEQ ID NO.: 44
PSf34.1e heavy chain variable sequence
EVQLEESGAELVKPGASVKLSCTASGFYIKDTYIHWVKQRPEEVLEWIGG
IGSADGDTRYDPKFQGKATITADTSSNSAYLHLTSLTSEDTVVYFCAREL
AYWGAWTTV
SEQ ID NO.: 45
PSf42.1 light chain variable sequence
DILMTQSPLTLSVIIGQPASFSCKSSHSLLHRDGRTYLNWLLQRPGQSPQ
RLIYLVSKLDSGVPDRFTGSGSGTDFTLKISRVEAEDLGVYYCWQGTHFP
RTFGGGTKLEIKRA
SEQ ID NO.: 46
PSf42.1 heavy chain variable sequence
EVQLEESGAEFVRPGAAVKLSCTVSGLNIKDSYLHWVKQRPEQGLEWIGG
IDPANGDVEYDPKFQGKAAITADTSSNTAYLRLSSLTSEDTAVYYCAPFP
YVVGAGTTVTVS
SEQ ID NO.: 47
PSf42.2 light chain variable sequence
CILMTQSPLSLPVSLGDQASISCRSSQSLVHSNGNTYLHWYLQKPGQSPK
FLIYKASNRFSGVPDRFSGRGSGTDFTLKISRVEAEDLGVYFCFQSTHVP
YTFGGGTKLEIKRA
SEQ ID NO.: 48
PSf42.2 heavy chain variable sequence
EVKLQQSGAELVKPGASVKLSCTASGFNIKDTYMHWVKQRPEQGLEWIGG
IDPADGEPLYDPKFQDKATITTDTSSNTVYLQISSLTSEDSPVYYCAPVR
SSFDYWGQGTTVTVS
SEQ ID NO.: 49
PSf42.3 light chain variable sequence
HSADPVSISCKSSQSLLHRDGKTYLNWVFQRPGQSPQRLIYLVSLVDSGV
PDRFTGSGSGTDFTLKINRVEAEDLGVYYCWQGTHFPRTFGGGTKLEIKR
A
SEQ ID NO.: 50
PSf42.3 heavy chain variable sequence
EVQLQQSGAELAKPGASVKLSCTGSGFNIKDTYMHWVKQRPEQGLEWIGG
IDPETGNTKFDPRFQDKATITSDTSSNTVLLQLSSLTSEDTAVYYCANLG
RPFAHWGQGTTVTVS
SEQ ID NO.: 51
PSf42.4 light chain variable sequence
DILMTQSPLTLSVIIGQPASFSCKSSHSLLHRDGRTYLNWLLQRPGQSPQ
RLIYLVSKLDSGVPDRFTGSGSGTDFTLKISRVEAEDLGVYYCWQGTHFP
RTFGGGTKLEIKRA
SEQ ID NO.: 52
PSf42.4 heavy chain variable sequence
EVKLQQSGAELVKPGASVKLSCTASGFSIRDTYMHWVRQRPEQGLEWITG
IDPENGNSKYAPRFQDKATIIADTSSNTVHLQLDTLTSEDTAVYYCTREL
AYWGQGTTVTVS
SEQ ID NO.: 53
PSf47.1 light chain variable sequence
DILMPQSPSSLAVSAGEKVTMSCKSSQSLLNSRTRKNYLAWYQQKLGQSP
KLLIYWASTRESGVPDRFTGSGSGTDFTLTISSVQAEDLAVYYCKQSYNF
ITFGAGTKLELKRA
SEQ ID NO.: 54
PSf47.1 heavy chain variable sequence
EVKLQESGPDLVKPGASVKVSCKASGYTFTVYVIHWVIQKPGQGLEWIGY
INPYNDGAEYNENFKGKATLTSDKSSSTAYMELSSLTSEDSAVYYCTRGE
NYYTSRYGFFDVWGQGTTVTVS
SEQ ID NO.: 55
human PSMA (accession No. NP004467)
MWNLLHETDSAVATARRPRWLCAGALVLAGGFFLLGFLFGWFIKSSNEAT
NITPKHNMKAFLDELKAENIKKFLYNFTQIPHLAGTEQNFQLAKQIQSQW
KEFGLDSVELAHYDVLLSYPNKTHPNYISIINEDGNEIFNTSLFEPPPPG
YENVSDIVPPFSAFSPQGMPEGDLVYVNYARTEDFFKLERDMKINCSGKI
VIARYGKVFRGNKVKNAQLAGAKGVILYSDPADYFAPGVKSYPDGWNLPG
GGVQRGNILNLNGAGDPLTPGYPANEYAYRRGIAEAVGLPSIPVHPIGYY
DAQKLLEKMGGSAPPDSSWRGSLKVPYNVGPGFTGNFSTQKVKMHIHSTN
EVTRIYNVIGTLRGAVEPDRYVILGGHRDSWVFGGIDPQSGAAWHEIVRS
FGTLKKEGWRPRRTILFASWDAEEFGLLGSTEWAEENSRLLQERGVAYIN
ADSSIEGNYTLRVDCTPLMYSLVHNLTKELKSPDEGFEGKSLYESWTKKS
PSPEFSGMPRISKLGSGNDFEVFFQRLGIASGRARYTKNWETNKFSGYPL
YHSVYETYELVEKFYDPMFKYHLTVAQVRGGMVFELANSIVLPFDCRDYA
VVLRKYADKIYSISMKHPQEMKTYSVSFDSLFSAVKNFTEIASKFSERLQ
DFDKSNPIVLRMMNDQLMFLERAFIDPLGLPDRPFYRHVIYAPSSHNKYA
GESFPGIYDALFDIESKVDPSKAWGEVKRQIYVAAFTVQAAAETLSEVA
SEQ ID NO.: 56
NH₂-CGKSLYESWTKK
SEQ ID NO.: 57
TGAGGTGCAGCTGGAGGAGTC
SEQ ID NO.: 58
GTGACCGTGGTCCCTGCGCCCCAG
SEQ ID NO.: 59
GACATTCTGATGACCCAGTCT
SEQ ID NO.: 60
TTTTATTTCCAGCTTGGTCCC
SEQ ID NO: 61
(CDRL1 consensus)
X₁ₐSSX₂ₐSLX₃ₐX₄ₐX₅ₐX₆ₐX₇ₐX₈ₐX₉ₐX₁₀ₐYLX₁₁ₐ

wherein

X₁ₐ is a basic amino acid (eg. arginine, lysine)

X₂ₐ is glutamine or histidine

X₃ₐ is an hydrophobic amino acid (eg. valine, leucine)

X₄ₐ is a asparagine or histidine

X₅ₐ is serine or arginine

X₆ₐ is absent or arginine

X₇ₐ is aspartic acid, asparagine or threonine

X₈ₐ is glycine or arginine

X₉ₐ is a basic amino acid (eg. arginine, lysine) or asparagine.

X₁₀ₐ is threonine or asparagine)

X₁₁ₐ is asparagine, histidine or alanine

SEQ ID NO: 62
(CDRL2 consensus 1)
LVSX_{1b}X_{2b}DX_{3b}

Wherein X_{1b} is a basic amino acid (arginine or lysine) or leucine

X_{2b} is an hydrophobic amino acid (leucine or valine)

X_{3b} is serine or absent

SEQ ID NO: 63
(CDRL2 consensus 2)
X_{1c}ASX_{2c}RX_{3c}S

Wherein X_{1c} is lysine or trytophan

X_{2c} is asparagine and threonine

X_{3c} is phenylalanine or glutamic acid

SEQ ID NO: 64
(CDRL3 consensus)
X_{1d}QX_{2d}THX_{3d}PX_{4d}T

Wherein X_{1d} is an aromatic amino acid (eg. Phenylalanine or tryptophan)

X_{2d} is serine or glycine

X_{3d} is phenylalanine or valine

X_{4d} is arginine or tyrosine

SEQ ID NO: 65
(CDRH1 consensus 1)
GX₁ₑX₂ₑX₃ₑX₄ₑX₅ₑX₆ₑX₇ₑX₈ₑH

Wherein X₁ₑ is an hydrophobic amino acid (phenylalanine or leucine) or tyrosine

X₂ₑ is asparagine, serine, tyrosine or threonine

X₃ₑ is an hydrophobic amino acid (phenylalanine or isoleucine)

X₄ₑ is a basic amino acid (lysine, arginine) or threonine

X₅ₑ is valine or aspartic acid

X₆ₑ is an hydrophilic amino acid (eg. Threonine or serine) or tyrosine

X₇ₑ is tyrosine or valine

X₈ₑ is an hydrophobic amino acid (methionine, isoleucine or leucine)

SEQ ID NO: 66
(CDRH1 consensus 2)
GX_{1f}X_{2f}IX_{3f}DX_{4f}YX_{5f}H

Wherein

X_{1f} is an hydrophobic amino acid (phenylalanine or leucine)

X_{2f} is asparagine, serine or tyrosine

X_{3f} is a basic amino acid (lysine or arginine)

X_{4f} is an hydrophilic amino acid (eg. serine or threonine)

X_{5f} is an hydrophobic amino acid (eg. Leucine, isoleucine or methionine)

SEQ ID NO: 67
(CDRH2 consensus 1)
GIX_{1g}X_{2g}X_{3g}X_{4g}GX_{5g}X_{6g}X_{7g}

Wherein X_{1g} is aspartic acid or glycine

X_{2g} is proline or serine

X_{3g} is alanine or glutamic acid

X_{4g} is threonine, asparagine or aspartic acid

X_{5g} is a aspartic acid, glutamic acid or asparagine

X_{6g} is threonine, serine, valine or proline

X_{7g} is a basic amino acid (lysine or arginine), glutamic acid or leucine

SEQ ID NO: 68
(CDRH2 consensus 2)
GIDPEX₁ₕGNX₂ₕK

Wherein X₁ₕ is threonine or arginine

X₂ₕ is a neutral hydrophilic amino acid (threonine or serine)

SEQ ID NO: 69
(heavy chain)
LGQLQQSGAELVKPGASVKLSCTGSGFNIKDTYMHWVKQRPEQGLEWIGG
IDPENGNTKFDPRFQDKATITADASSNTVLLQLSSLTSEDTAVYYCANLG
RPFAHWGQGTTVTSS
SEQ ID NO: 70
GIDPENGNTK

### REFERENCES

### Patent References

(1) US2004/0115209
(2) US2006/0189515

### Non-Patent References

(1) De Crescenzo G, Grothe S, Tsang M, Zwaagstra J, and O'Connor-McCourt M D: Real-time monitoring of the interactions of transforming growth factor-beta (TGF-beta) isoforms with latency-associated protein and the ectodomains of the TGF-beta type II and III receptors reveals different kinetic models and stoichiometries of binding. J Biol Chem 2001; 276:29632-29643.
(2) Bioconjugate Techniques (1996) Elsevier Science (USA)
(3) Guillemard V, Saragovi H U: Taxane-antibody conjugates afford potent cytotoxicity, enhanced solubility, and tumor target selectivity. Cancer Research 61: 694-699, 2001.

1-99. (canceled)

100. An antibody or antigen binding fragment thereof comprising:
a. A light chain variable region having at least 80% sequence identity with SEQ ID NO.:38 and complementary determining regions comprising a CDRL1 as defined in SEQ ID NO.:1, a CDRL2 as defined in SEQ ID NO.:2 and a CDRL3 as defined in SEQ ID NO.:3 and a heavy chain variable region having at least 80% sequence identity with SEQ ID NO.: 39 and complementary determining regions comprising a CDRH1 as defined in SEQ ID NO.:4, a CDRH2 as defined in SEQ ID NO.:5 or 6 and a CDRH3 as defined in SEQ ID NO.:7 or;
b. A light chain variable region having at least 80% sequence identity with SEQ ID NO.:49 and complementary determining regions comprising a CDRL1 as defined in SEQ ID NO.:20, a CDRL2 as defined in SEQ ID NO.:21 and a CDRL3 as defined in SEQ ID NO.:22 and a heavy chain variable region having at least 80% sequence identity with SEQ ID NO.:50 and complementary determining regions comprising a CDRH1 as defined in SEQ ID NO.:23, a CDRH2 as defined in SEQ ID NO.:24 and a CDRH3 as defined in SEQ ID NO.:25.

101. The antibody or antigen binding fragment thereof of claim 100, comprising a light chain variable region identical to SEQ ID NO.:38 and a heavy chain variable region identical to SEQ ID NO.:39.

102. The antibody or antigen binding fragment thereof of claim 100, comprising a light chain variable region identical to SEQ ID NO.:49 and a heavy chain variable region identical to SEQ ID NO.:50.

103. A pharmaceutical composition comprising the antibody or antigen binding fragment of claim 100 and a pharmaceutically acceptable carrier.

104. The pharmaceutical composition of claim 103, further comprising an anticancer drug.

105. A conjugate comprising the antibody or antigen binding fragment of claim 100 and a detectable moiety or a therapeutic moiety.

106. A pharmaceutical composition comprising the conjugate of claim 105 and a pharmaceutically acceptable carrier.

107. A method for reducing the growth of a prostate specific membrane antigen (PSMA)-expressing cell, the method comprising administering the antibody or antigen-binding fragment of claim 100 to a subject in need.

108. The method of claim 107, wherein the PSMA-expressing cell is a tumor cell.

109. The method of claim 108, wherein the tumor cell is a prostate tumor cell.

110. The method of claim 107, wherein the antibody or antigen binding fragment is naked.

111. The method of claim 107, wherein the antibody or antigen binding fragment is in combination with a cytotoxic drug or is conjugated with a cytotoxic drug.

112. A method for detecting a prostate specific membrane antigen (PSMA)-expressing cell, the method comprising administering the antibody or antigen-binding fragment of claim 100 to a subject in need.

113. The method of claim 112, wherein the PSMA-expressing cell is a tumor cell.

114. The method of claim 112, wherein the PSMA-expressing cell is a neovasculature cell.

115. The method of claim 112, wherein the antibody or antigen-binding fragment is conjugated with a detectable moiety.
